SEQUENCE LISTING (54 sequences)

SEQ ID NO: 1 - DNA, 1218 bp, Bacillus subtilis
atgctgtcagtcatgttagggccaggcgctactgaagttttggcagcaagtgatgtaacagttaatgtatctgcagagaaacaagtgattcgcggttttggagggatgaatcatccggcttgggctggggatcttacagcagctcaaagagaaactgcttttggcaatggacagaaccagttaggattttcaatcttaagaattcatgtagatgaaaatcgaaataattggtataaagaggtggagactgcaaagagtgcggtcaaacacggagcaatcgtttttgcttctccttggaatcctccaagtgatatggttgagacctttaatcggaatggtgacacatcggctaaacggctgaaatacaacaagtacgcagcatacgcgcagcatcttaacgattttgttaccttcatgaagaataatggtgtgaatctttacgcgatttcggtccaaaacgagcctgattacgctcacgagtggacgtggtggacgccgcaagaaatacttcgctttatgagagaaaacgccggctcgatcaatgcccgcgtcattgcgcctgagtcatttcaatacttgaagaatttgtcggacccgatcttgaacgatccgcaggctcttgccaatatggatattctcggaactcacctgtacggcacccaggtcagccaattcccttatcctcttttcaaacaaaaaggagcggggaaggacctttggatgacggaagtatactatccaaacagtgataccaactcggcggatcgatggcctgaggcattggatgtttcacagcatattcacaatgcgatggtagagggggactttcaagcttatgtatggtggtacatccgaagatcatatggacctatgaaagaagatggtacgatcagcaaacgcggctacaatatggctcatttctcaaagtttgtgcgtcccggctatgtaaggattgatgcaacgaaaaaccctaatgcgaacgtttacgtgtcagcctataaaggtgacaacaaggtcgttattgttgccatcaataaaagcaacacaggagtcaaccaaaactttgttttgcagaatggatctgcttcaaacgtatctagatggatcacgagcagcagcagcaatctacaacctggaacgaatctcactgtatcaggcaatcatttttgggctcatcttccagctcaaagcgtgacaacatttgttgtaaatcgttaa

SEQ ID NO: 2 - protein, 405 aa, Bacillus subtilis
MLSVMLGPGATEVLAASDVTVNVSAEKQVIRGFGGMNHPAWAGDLTAAQRETAFGNGQNQLGFSILRIHVDENRNNWYKEVETAKSAVKHGAIVFASPWNPPSDMVETFNRNGDTSAKRLKYNKYAAYAQHLNDFVTFMKNNGVNLYAISVQNEPDYAHEWTWWTPQEILRFMRENAGSINARVIAPESFQYLKNLSDPILNDPQALANMDILGTHLYGTQVSQFPYPLFKQKGAGKDLWMTEVYYPNSDTNSADRWPEALDVSQHIHNAMVEGDFQAYVWWYIRRSYGPMKEDGTISKRGYNMAHFSKFVRPGYVRIDATKNPNANVYVSAYKGDNKVVIVAINKSNTGVNQNFVLQNGSASNVSRWITSSSSNLQPGTNLTVSGNHFWAHLPAQSVTTFVVNR

SEQ ID NO: 3 - DNA, 1263 bp, Bacillus licheniformis
atgaaaaaattaatttgtgtattgttggcatatgtaacgatgctgtcgctcatgttaaccgggcctgccgcttctgaagtttcagcagccagtgacgcaacagttcgtctatctgcagaaaaacaagtgattcgcggttttggagggatgaatcacccggcttggatcggggatcttacagcagctcaaagagaaaccgcctttggcaatggacagaatcagttaggcttttcaattttaagaattcatgttgatgaaaatagaaacaattggtacagagaagtggagacggcaaagagtgcgatcaaacacggagcaatcgtttttgcttctccctggaatccgccaagcgatatggttgagactttcaatcggaatggagacacatcagctaaacggctgagatacgataagtacgccgcatacgcgaagcatcttaacgactttgttaccttcatgaaaaataatggcgtgaatctgtatgcgatttccgtccaaaacgagcctgattacgcacacgactggacgtggtggacaccgcaagaaatacttcgctttatgaaagagaacgccggctcgattaatgcccgtgtcatcgcgcctgagtcgtttcaatacttaaaaaatatatcggatccgattttgaatgatccgaaggcgcttgccaatatggatattcttggcgctcatctttatggtacacagcttaacaatttcgcttatccactgttcaaacaaaaaggagcaggaaaagatctttggatgacggaagtatattatccgaacagtgacaataattctgcggaccgctggcctgaggcattggatgtttcgcaccatattcacaattcgatggtagagggagattttcaggcttatgtatggtggtacatccgcagatcatacggtcctatgaaagaagacggtacgatcagcaagcgcggttacaatatggctcatttctcgaagtttgtccgtcccggttatgtcagggttgatgcgacaaagagccctgcttcaaacgtttacgtatctgcctataaaggtgacaacaaagtcgttatagttgccattaataaaaacaactcaggggttaaccaaaacttcgttctgcagaatggatctgtttctcaagtatccagatggatcacgagcagcagcagcaaccttcaacctggaacgaatctcaatgtaacagacaatcatttttgggcgcatcttccggctcagagtgtgacaacatttgtcgctaacctccgctaa

SEQ ID NO: 4 - protein, 420 aa, Bacillus licheniformis
MKKLICVLLAYVTMLSLMLTGPAASEVSAASDATVRLSAEKQVIRGFGGMNHPAWIGDLTAAQRETAFGNGQNQLGFSILRIHVDENRNNWYREVETAKSAIKHGAIVFASPWNPPSDMVETFNRNGDTSAKRLRYDKYAAYAKHLNDFVTFMKNNGVNLYAISVQNEPDYAHDWTWWTPQEILRFMKENAGSINARVIAPESFQYLKNISDPILNDPKALANMDILGAHLYGTQLNNFAYPLFKQKGAGKDLWMTEVYYPNSDNNSADRWPEALDVSHHIHNSMVEGDFQAYVWWYIRRSYGPMKEDGTISKRGYNMAHFSKFVRPGYVRVDATKSPASNVYVSAYKGDNKVVIVAINKNNSGVNQNFVLQNGSVSQVSRWITSSSSNLQPGTNLNVTDNHFWAHLPAQSVTTFVANLR

SEQ ID NO: 5 - DNA, 1272 bp, Bacillus amyloliquefaciens FZB42
atgatgtcgagcgtaaaaaaaacaatttgtgtattattggtatgtttcacaatgctgtccgtcatgctcttaggaccgggagttactgaagtttcagcagcaagtgatgcaacagttaatatatctgcagaaagacaagtgattcgcggttttggaggaatgaaccacccggcttggattggagacttgactgcggctcaacgggagaccgcttttggcaatggacagaatcagttaggtttttcggtcttaagaattcatgtagatgaaaatagaaataattggtacaaggaagtggagactgcaaagagtgcgatcaaacatggagcaatcgtttttgcttccccttggaatccgccaaacgatatggttgagactttcaatcataatggtgacacaacagctaagcggctgagatacgataagtacgccgcatacgcgcagcatcttaacgatttcgttaatttcatgaaaagtaatggtgtgaatctgtatgcgatttctatgcaaaacgagcctgattacgctcacgaatggacatggtggacgccccaagaaatcctgcgtttcatgagagagaatgccggttccattaatgctcgtgtgattgcgccggaatcatttcaatacctgaaaaatatatcggaccccattttgaacgatccgcaggcgcttaggaatatggatatcctcggaacccacctgtacggcactcaggtcagtcagtttccttatcctcttttcaaacaaaaaggaggagggaaagagctatggatgacggaggtatactatccgaatagtgacaacaattcagcggatcgctggcctgaggctttaggtgtttcagagcatattcatcattcaatggtagagggagattttcaagcttacgtgtggtggtacatccgcagatcatacggccctatgaaagaagatgggatgatcagcaaacgcggatacaatatggctcatttctcaaaatttgtgcgccctggctatgtaaggattgatgcaacgaaaaatcctgaaccgaatgtttacgtgtcagcctataaaggagacaataaggtcgtgattgttgccattaataaaaataacacaggggtcaaccaaaacttcgtattgcagaatggaactgcttcgcaagtatccagatggatcacgagcagcagcagcaatcttcaacctggaacggatctcaaagtaacggacaatcatttttgggcccatctgccggctcaaagcgtgacaacatttgttgtaaagcgttaa

SEQ ID NO: 6 - protein, 423 aa, Bacillus amyloliquefaciens FZB42
MMSSVKKTICVLLVCFTMLSVMLLGPGVTEVSAASDATVNISAERQVIRGFGGMNHPAWIGDLTAAQRETAFGNGQNQLGFSVLRIHVDENRNNWYKEVETAKSAIKHGAIVFASPWNPPNDMVETFNHNGDTTAKRLRYDKYAAYAQHLNDFVNFMKSNGVNLYAISMQNEPDYAHEWTWWTPQEILRFMRENAGSINARVIAPESFQYLKNISDPILNDPQALRNMDILGTHLYGTQVSQFPYPLFKQKGGGKELWMTEVYYPNSDNNSADRWPEALGVSEHIHHSMVEGDFQAYVWWYIRRSYGPMKEDGMISKRGYNMAHFSKFVRPGYVRIDATKNPEPNVYVSAYKGDNKVVIVAINKNNTGVNQNFVLQNGTASQVSRWITSSSSNLQPGTDLKVTDNHFWAHLPAQSVTTFVVKR

SEQ ID NO: 7 - DNA, 1263 bp, Bacillus safensis
atgtccataatcaaaaaaccaatttgtactttattgatctgcttcactatgctgtctgtcatgttcatagggcctggggtaactgaggtttcagcagcaagtgatgcgaatattaatgtcaatgcggaaagacaagtgattcgcggctttggcggaatgaaccacccggcttggattggtgatttaaccgcccctcaaagggaaaccgcctttggcaatgggcagaatcaattaggattctccattctacgaattaatgtagacgagaacagaaataattggcacagagaagttgctaccgccaaaagagcaatagagcatggcgctttagtgatcgcttcgccttggaatcctccaagccatatggtggagactttcaaccgtaatggtgcgtctgcaaagcggttgagatacaatcaatacgccgcgtatgctcagcatctcaatgattttgtgacatacatgaaaaataatggtgtcaacctctatgccatttctgtacaaaacgagcctgattatgcacacgaatggacatggtggacgcctcaggaaatcctgcgattcatgagagaaaatgccggctccattaatgcacgcgtgattgcaccagaatcttttcaataccttaaaaatatatcagatcctatcttaaacgatccgcaggcgcttagaaatatggacattctcggtgcccatctgtatggaacccagatcagccagcttccgtatcctctttttaaacaaaaaggagcggggaaagagctgtggatgacagaggtatattacccgaatagtgataacaattcagcggaccgctggcctgaggcattaggggtgtcagagcatattcaccattcgatggtggaaggtgattttcaggcgtatgtttggtggtacatccgcagatcatacggtcctatgaaggaagatggaatgattagcaaacgtggctacaacatggcgcatttctccaagtttgtgcgtccagggtacgtcaggattgatgcaacgaaaagccctgaaccgaatgttttcgtctcagcctataaaggggacaataaggtcgtcattgtagcgattaataaaaacaatacaggcgttaaccagcactttgtcatgcaaaatggaaccgcttcacaagcgtcaagatggattacgagtagtaacagcaaccttcagcctggaactgacttaaatatatcaggtaatcaattttgggctcatctcccggctcaaagtgtgacaacatttgtggtcaaacgc

SEQ ID NO: 8 - protein, 421 aa, Bacillus safensis
MSIIKKPICTLLICFTMLSVMFIGPGVTEVSAASDANINVNAERQVIRGFGGMNHPAWIGDLTAPQRETAFGNGQNQLGFSILRINVDENRNNWHREVATAKRAIEHGALVIASPWNPPSHMVETFNRNGASAKRLRYNQYAAYAQHLNDFVTYMKNNGVNLYAISVQNEPDYAHEWTWWTPQEILRFMRENAGSINARVIAPESFQYLKNISDPILNDPQALRNMDILGAHLYGTQISQLPYPLFKQKGAGKELWMTEVYYPNSDNNSADRWPEALGVSEHIHHSMVEGDFQAYVWWYIRRSYGPMKEDGMISKRGYNMAHFSKFVRPGYVRIDATKSPEPNVFVSAYKGDNKVVIVAINKNNTGVNQHFVMQNGTASQASRWITSSNSNLQPGTDLNISGNQFWAHLPAQSVTTFVVKR

SEQ ID NO: 9 - DNA, 1341 bp, Paenibacillus macerans
atgatttcacggtttaagaaagctatttgtgcaggttcggcccttgtaatagcattgaccctcatggtagcgccgggcgatgttgccgcagccagcgacgccgtcgtgaatgtatcggcggaaaaacaagtgattcgcggtttcggaggcatcaaccacccggcttggatcggagatttgacggcggcacaaagagaaaccgctttcggaaacggaaataaccagttaggtttctcgatcttaagaatctatgtgcatgatgaccgcaatcagtggtaccgggaattggaaacggccaaacgagccatcgccctcggagccatcgtgtttgcgtcgccatggaacccgcccgccgacatggtagagaccttcaaccgcaacggtgatacgtcggcaaaacggctccgttacgacaagtacgctgcgtatgcccagcaccttaatgatttcgtgacttatatgaggaataatggcgtgaatctgtatgcgatttccgtacaaaacgaacccgattatgcgcatgactggacgtggtggactccccaggaaatgcttcgcttcatgaaagaaaacgccggttccattaatgccagagtgatcgcgccggaatcgttccagtatctgaaaaatatgtcggacccgattttgaacgattcgcaggcgcttgccaatatggatattcttggtgcgcatctatacggtacccagatcagcaatttcgcttatccgctattcaaacaaaaaggagcgggaaaagaactctggatgacggaagtatattacccgaacagcgacaacaactcggcggatcgctggcccgaagccttggatgtgtcctatcatatccacaatgcgatggtagagggagattttcaggcttacgtatggtggtatatccgcagatcatacggtccgatgaaggaagacggcacaatcagcaaacgcggttataatatggctcatttctccaaattcgtccgtcccggctatgttagggtggatgccacgaaaaatcctgaaacgaacgtttacgtatctgcctataaaggtaacaacaaaatcgttatcgttgccattaacaggagcggctccggggttaatcagaactttgtcctgcggaatggatcggtttcgaaagtatcccgatggatcacgaacagcagcagcaatcttcaacccggaacggagcttacggtgacgggcgagaatttctgggctcatctcccagcccaaagcgtgaccaccttcgtagctgatcttggtacagcttcaggcagaagcgcagccaatgaagctgagacagacacaaccttgcctgacgctgtcgtagataacctacgt

SEQ ID NO: 10 - protein, 447 aa, Paenibacillus macerans
MISRFKKAICAGSALVIALTLMVAPGDVAAASDAVVNVSAEKQVIRGFGGINHPAWIGDLTAAQRETAFGNGNNQLGFSILRIYVHDDRNQWYRELETAKRAIALGAIVFASPWNPPADMVETFNRNGDTSAKRLRYDKYAAYAQHLNDFVTYMRNNGVNLYAISVQNEPDYAHDWTWWTPQEMLRFMKENAGSINARVIAPESFQYLKNMSDPILNDSQALANMDILGAHLYGTQISNFAYPLFKQKGAGKELWMTEVYYPNSDNNSADRWPEALDVSYHIHNAMVEGDFQAYVWWYIRRSYGPMKEDGTISKRGYNMAHFSKFVRPGYVRVDATKNPETNVYVSAYKGNNKIVIVAINRSGSGVNQNFVLRNGSVSKVSRWITNSSSNLQPGTELTVTGENFWAHLPAQSVTTFVADLGTASGRSAANEAETDTTLPDAVVDNLR

SEQ ID NO: 11 - DNA, 1563 bp, Paenibacillus cookii DSM 16944
gcaagcgatgcaacgatcaatttggcggccgagaagcaagtgatccgcgggttcggaggaatcaaccatccggtatgggcgggagacttgacggcagcgcaacgggaaaccgcatttggcaacggggacaaccaattgggcttctccgttttaagaattcatgtggatgaagatcggaatcagtggagcaaggaagtcgaaacggccaaaagcgcgatcgcgcgcggagccatcgtttttgcttcgccttggaatcccccaagcgatatgaccgagaccttcaatcgtaacggagatacatcggccaaacggctcagatacgataaatacgctgcctatgcgcagtatctcaacgattttgttacgtatatgaaaaataacggcgtcgatttgtatgcgatttcggtccaaaacgaacctgactatgcgcatacctggacgtggtggacgccggcagaaatgcttcgctttatgaaagaaaatgccggatccatccaatgccgggtaatcgctcccgagtcattttcttatctgaaaaacatgtcggacccgattctgaatgatccgcaggcccttgccaacatggatattctgggcgcccatctgtacggcactccgttcgataacttttcttatcctctattcaaagaaaaaggagcggggaaggatctctggatgacggaggtctattaccccaatagcgataataattcggcggatcgttggccggaagcgctggatgtttcctatcatattcacaaagcgatggccaagggggattttcaggcttacgtatggtggtatatccgcaggcagtacggtccgatcaaggaggatggttcgataagcaagcggggttacaacatggctcatttctccaagtttgttcgccctggttacgtgaggatcgatgcaacggagaatccggataccgacgtatatacatccgcctataaaggcgacaacaaggttgtggtcgtggcgatcaacaggggcacttcggccaaaagccaacatttcgtcctgcaaaacggaacggcatccaaggtatcttcatgggtgacggacgccggccgcaatctggcccccgggtccgttcatacgtccggagattcatttacggcgcagcttccagcccagagcgtgaccacgtttgtggttgacttgggcagcaacggcagcacttacgaagcagaaagcggcacgacattgaccgatgcggtcgttgaaaccgtcaatcccggatatcacggcaccggatacgttaactttaatgcctcatccggcgcagccgttcagtggaacggtatttattgcgcagtggccggaaccaaaaacgtggattttcgttacgcgctggaatccggttcaagaaaagtggatgtttacgtcaacgggacaaaagcgatcagcaatgctgagtttacagcgaccggcagctggtccgcctggagaaatcaaacgattcaagtatccatgaacagcggaatcaacacgttaaaagtcgttacaaccgggacggaagggccgaatatggacagcgttaccgtttcgccaggttcg

SEQ ID NO: 12 - protein, 555 aa, Paenibacillus cookii DSM 16944
MSRFKKPVCAWLACCTMLSLIFSVSVSVPNKALAASDATINLAAEKQVIRGFGGINHPVWAGDLTAAQRETAFGNGDNQLGFSVLRIHVDEDRNQWSKEVETAKSAIARGAIVFASPWNPPSDMTETFNRNGDTSAKRLRYDKYAAYAQYLNDFVTYMKNNGVDLYAISVQNEPDYAHTWTWWTPAEMLRFMKENAGSIQCRVIAPESFSYLKNMSDPILNDPQALANMDILGAHLYGTPFDNFSYPLFKEKGAGKDLWMTEVYYPNSDNNSADRWPEALDVSYHIHKAMAKGDFQAYVWWYIRRQYGPIKEDGSISKRGYNMAHFSKFVRPGYVRIDATENPDTDVYTSAYKGDNKVVVVAINRGTSAKSQHFVLQNGTASKVSSWVTDAGRNLAPGSVHTSGDSFTAQLPAQSVTTFVVDLGSNGSTYEAESGTTLTDAVVETVNPGYHGTGYVNFNASSGAAVQWNGIYCAVAGTKNVDFRYALESGSRKVDVYVNGTKAISNAEFTATGSWSAWRNQTIQVSMNSGINTLKVVTTGTEGPNMDSVTVSPGS

SEQ ID NO: 13 - DNA, 1176 bp, Paenibacillus tundrae DSM 21291
gccagcgatgttactgtaaacttgtcttctcaaaagcagcttatcaagggatttgggggaatcaatcatccggcctggattggagatttgacaccagcgcaaagagatacagcatttggtaatggacaaaatcagctgggtttttcaattttgcgtgtgtatattgatgataataaaaacaattggtataaagaagtggctaccgcgaaacgagcgattgaacaaggggctatcgtctttgcgtcaccatggaatcccccaagtgatatggtggagacattcaatcgtaatggcgacaccacggccaaacgacttaaatacgataaatatgctgcgtattcacagcatcttaatgactttgtttcttacatgaaatccaacggtgttaatctctatgcaatctccgtacaaaatgaaccggattatgcgcatgattggacgtggtggacgcctcaggagatgcttcgattcatgaaagattatgcaggctccattacaggagcgaaagtaatggcgccagaatccttctcctatctaaaagaaatgtcagaccccatactgaatgatcctcaagctttagcaaacatggacattttgggtgcacatacgtacggtacacaatttaacaatttcccttacccactttttaaacaaaaaggtgcaggaaaggaactttggatgtcagaggtatattaccctaatagtaacgcgaactcagcagataattggccagaagcgctggatgtctcctatcacatccataatgcgatggtagaggcagatttccaggcgtatgtatggtggtatattcgtagacagtacggtcccatgaaagaggatggcaccataagcaaacggggatataacatggcccatttttccaagtttgttcgaccaggctttgtaagagttgaggcgacgaaaaatccggatacccagacgtttatctctgcttacaaaggagacaacaaggtggttatcgtagcaatcaaccgaggtacttccgccgtaaaccaaaaatttgtattgcagaacgggaacgcctcaaccgtatcttcctggattacggacagcacaagaaatctggctgccggctcttcgataaacgtcactggcaattcctttaccgctcaacttcctgcccagagtgttaccacattcacagctccattaaaa

SEQ ID NO: 14 - protein, 422 aa, Paenibacillus tundrae DSM 21291
LLFKLKKSISILLALLTALPLILTPIQASAASDVTVNLSSQKQLIKGFGGINHPAWIGDLTPAQRDTAFGNGQNQLGFSILRVYIDDNKNNWYKEVATAKRAIEQGAIVFASPWNPPSDMVETFNRNGDTTAKRLKYDKYAAYSQHLNDFVSYMKSNGVNLYAISVQNEPDYAHDWTWWTPQEMLRFMKDYAGSITGAKVMAPESFSYLKEMSDPILNDPQALANMDILGAHTYGTQFNNFPYPLFKQKGAGKELWMSEVYYPNSNANSADNWPEALDVSYHIHNAMVEADFQAYVWWYIRRQYGPMKEDGTISKRGYNMAHFSKFVRPGFVRVEATKNPDTQTFISAYKGDNKVVIVAINRGTSAVNQKFVLQNGNASTVSSWITDSTRNLAAGSSINVTGNSFTAQLPAQSVTTFTAPLK

SEQ ID NO: 15 - DNA, 1269 bp, artificial sequence
  nucleotide sequence of the BsuGH30 gene from plasmid p2JM-BsuGH30
gtgagaagcaaaaaattgtggatcagcttgttgtttgcgttaacgttaatctttacgatggcgttcagcaacatgagcgcgcaggctgctggaaaagcaagtgatgtaacagttaatgtatctgcagagaaacaagtgattcgcggttttggagggatgaatcatccggcttgggctggggatcttacagcagctcaaagagaaactgcttttggcaatggacagaaccagttaggattttcaatcttaagaattcatgtagatgaaaatcgaaataattggtataaagaggtggagactgcaaagagtgcggtcaaacacggagcaatcgtttttgcttctccttggaatcctccaagtgatatggttgagacctttaatcggaatggtgacacatcggctaaacggctgaaatacaacaagtacgcagcatacgcgcagcatcttaacgattttgttaccttcatgaagaataatggtgtgaatctttacgcgatttcggtccaaaacgagcctgattacgctcacgagtggacgtggtggacgccgcaagaaatacttcgctttatgagagaaaacgccggctcgatcaatgcccgcgtcattgcgcctgagtcatttcaatacttgaagaatttgtcggacccgatcttgaacgatccgcaggctcttgccaatatggatattctcggaactcacctgtacggcacccaggtcagccaattcccttatcctcttttcaaacaaaaaggagcggggaaggacctttggatgacggaagtatactatccaaacagtgataccaactcggcggatcgatggcctgaggcattggatgtttcacagcatattcacaatgcgatggtagagggggactttcaagcttatgtatggtggtacatccgaagatcatatggacctatgaaagaagatggtacgatcagcaaacgcggctacaatatggctcatttctcaaagtttgtgcgtcccggctatgtaaggattgatgcaacgaaaaaccctaatgcgaacgtttacgtgtcagcctataaaggtgacaacaaggtcgttattgttgccatcaataaaagcaacacaggagtcaaccaaaactttgttttgcagaatggatctgcttcaaacgtatctagatggatcacgagcagcagcagcaatctacaacctggaacgaatctcactgtatcaggcaatcatttttgggctcatcttccagctcaaagcgtgacaacatttgttgtaaatcgttaa

SEQ ID NO: 16 - protein, 422 aa, artificial sequence
  amino acid sequence of the BsuGH30 full length recombinant protein expressed from plasmid p2JM-BsuGH30
MRSKKLWISLLFALTLIFTMAFSNMSAQAAGKASDVTVNVSAEKQVIRGFGGMNHPAWAGDLTAAQRETAFGNGQNQLGFSILRIHVDENRNNWYKEVETAKSAVKHGAIVFASPWNPPSDMVETFNRNGDTSAKRLKYNKYAAYAQHLNDFVTFMKNNGVNLYAISVQNEPDYAHEWTWWTPQEILRFMRENAGSINARVIAPESFQYLKNLSDPILNDPQALANMDILGTHLYGTQVSQFPYPLFKQKGAGKDLWMTEVYYPNSDTNSADRWPEALDVSQHIHNAMVEGDFQAYVWWYIRRSYGPMKEDGTISKRGYNMAHFSKFVRPGYVRIDATKNPNANVYVSAYKGDNKVVIVAINKSNTGVNQNFVLQNGSASNVSRWITSSSSNLQPGTNLTVSGNHFWAHLPAQSVTTFVVNR

SEQ ID NO: 17 - DNA, 1269 bp, artificial sequence
  nucleotide sequence of BliXyn1 gene from plasmid p2JM-BliXyn1
gtgagaagcaaaaaattgtggatcagcttgttgtttgcgttaacgttaatctttacgatggcgttcagcaacatgagcgcgcaggctgctggaaaagcatcagacgccacagttagactgtcagcagagaaacaggttattagaggctttggcggaatgaatcacccggcatggatcggcgatcttacagcagctcagagagagacggccttcggcaatggccaaaaccaactgggcttttcaattctgagaatccatgttgatgaaaatagaaataactggtacagagaagttgaaacagctaagagcgcaatcaaacatggagcaattgtctttgcatcaccttggaatccgccgagcgatatggtcgaaacatttaacagaaatggcgatacatcagccaaaagacttagatatgataaatacgcagcatacgcaaaacatctgaatgattttgtgacgtttatgaaaaacaacggagtcaatctttatgcaatttcagttcaaaatgaaccggattatgcacatgattggacatggtggacaccgcaagaaattcttagatttatgaaggagaatgcaggctcaattaatgcaagagttattgcaccggaatcatttcaatatctgaagaatatctcagatccgattcttaatgatccgaaagcactggctaatatggacatcctgggagcacacctttatggaacgcaactgaacaattttgcatatcctctgtttaagcagaaaggcgcaggcaaagatctgtggatgacagaagtgtattatccgaactcagacaataatagcgcagatagatggccggaagcacttgacgtgagccatcatattcataacagcatggttgagggcgattttcaagcatatgtttggtggtatattagaagatcatatggcccgatgaaagaggatggcacaattagcaaaagaggctacaatatggcgcacttctcaaaattcgttagacctggatacgttagagttgatgctacaaagtcaccggcaagcaatgtttatgtttcagcatataaaggcgacaacaaggtggttattgtggcgatcaataagaataattcaggcgtgaatcagaacttcgttctgcaaaacggctcagtttcacaagtttcaagatggattacatcatcaagctcaaaccttcaaccgggcacaaacctgaatgttacagataatcacttttgggctcatctgcctgcccagtcagttacaacatttgttgcgaacctgaga

SEQ ID NO: 18 - protein, 423 aa, artificial sequence
  amino acid sequence of the BliXyn1 full length recombinant protein expressed from plasmid p2JM-BliXyn1
MRSKKLWISLLFALTLIFTMAFSNMSAQAAGKASDATVRLSAEKQVIRGFGGMNHPAWIGDLTAAQRETAFGNGQNQLGFSILRIHVDENRNNWYREVETAKSAIKHGAIVFASPWNPPSDMVETFNRNGDTSAKRLRYDKYAAYAKHLNDFVTFMKNNGVNLYAISVQNEPDYAHDWTWWTPQEILRFMKENAGSINARVIAPESFQYLKNISDPILNDPKALANMDILGAHLYGTQLNNFAYPLFKQKGAGKDLWMTEVYYPNSDNNSADRWPEALDVSHHIHNSMVEGDFQAYVWWYIRRSYGPMKEDGTISKRGYNMAHFSKFVRPGYVRVDATKSPASNVYVSAYKGDNKVVIVAINKNNSGVNQNFVLQNGSVSQVSRWITSSSSNLQPGTNLNVTDNHFWAHLPAQSVTTFVANLR

SEQ ID NO: 19 - DNA, 1269 bp, artificial sequence
  nucleotide sequence of BamGh2 gene from plasmid p2JM-BamGh2
gtgagaagcaaaaaattgtggatcagcttgttgtttgcgttaacgttaatctttacgatggcgttcagcaacatgagcgcgcaggctgctggaaaagcaagtgatgcaacagttaatatatctgcagaaagacaagtgattcgcggttttggaggaatgaaccacccggcttggattggagacttgactgcggctcaacgggagaccgcttttggcaatggacagaatcagttaggtttttcggtcttaagaattcatgtagatgaaaatagaaataattggtacaaggaagtggagactgcaaagagtgcgatcaaacatggagcaatcgtttttgcttccccttggaatccgccaaacgatatggttgagactttcaatcataatggtgacacaacagctaagcggctgagatacgataagtacgccgcatacgcgcagcatcttaacgatttcgttaatttcatgaaaagtaatggtgtgaatctgtatgcgatttctatgcaaaacgagcctgattacgctcacgaatggacatggtggacgccccaagaaatcctgcgtttcatgagagagaatgccggttccattaatgctcgtgtgattgcgccggaatcatttcaatacctgaaaaatatatcggaccccattttgaacgatccgcaggcgcttaggaatatggatatcctcggaacccacctgtacggcactcaggtcagtcagtttccttatcctcttttcaaacaaaaaggaggagggaaagagctatggatgacggaggtatactatccgaatagtgacaacaattcagcggatcgctggcctgaggctttaggtgtttcagagcatattcatcattcaatggtagagggagattttcaagcttacgtgtggtggtacatccgcagatcatacggccctatgaaagaagatgggatgatcagcaaacgcggatacaatatggctcatttctcaaaatttgtgcgccctggctatgtaaggattgatgcaacgaaaaatcctgaaccgaatgtttacgtgtcagcctataaaggagacaataaggtcgtgattgttgccattaataaaaataacacaggggtcaaccaaaacttcgtattgcagaatggaactgcttcgcaagtatccagatggatcacgagcagcagcagcaatcttcaacctggaacggatctcaaagtaacggacaatcatttttgggcccatctgccggctcaaagcgtgacaacatttgttgtaaagcgttaa

SEQ ID NO: 20 - protein, 421 aa, artificial sequence
  amino acid sequence of the BamGh2  full length recombinant protein expressed from plasmid p2JM-BamGh2
MRSKKLWISLLFALTLIFTMAFSNMSAQAAGKASDATVNISAERQVIRGFGGMNHPAWIGDLTAAQRETAFGNGQNQLGFSVLRIHVDENRNNWYKEVETAKSAIKHGAIVFASPWNPPNDMVETFNHNGDTTAKRLRYDKYAAYAQHLNDFVNFMKSNGVNLYAISMQNEPDYAHEWTWWTPQEILRFMRENAGSINARVIAPESFQYLKNISDPILNDPQALRNMDILGTHLYGTQVSQFPYPLFKQKGGGKELWMTEVYYPNSDNNSADRWPEALGVSEHIHHSMVEGDFQAYVWWYIRRSYGPMKEDGMISKRGYNMAHFSKFVRPGYVRIDATKNPEPNVYVSAYKGDNKVVIVAINKNNTGVNQNFVLQNGTASQVSRWITSSSSNLQPGTDLKVTDNHFWAHLPAQSVTTFVVK

SEQ ID NO: 21 - DNA, 1266 bp, artificial sequence
  nucleotide sequence of BsaXyn1 gene from plasmid p2JM (aprE- BsaXyn1)
gtgagaagcaaaaaattgtggatcagcttgttgtttgcgttaacgttaatctttacgatggcgttcagcaacatgagcgcgcaggctgctggaaaagcttcagacgcgaacatcaatgttaacgcagaaagacaagttattagaggctttggaggcatgaatcatccggcatggattggcgatcttacagccccgcaaagagaaacagcattcggcaacggccaaaaccaactgggcttttcaatccttagaattaatgttgatgaaaatagaaataactggcatagagaagttgctacagcgaagagagcaattgaacatggagcactggttatcgcgtcaccgtggaatccgccgtcacatatggttgaaacatttaacagaaatggcgcctcagctaaaagactgagatataatcagtatgcagcatacgcacaacatctgaacgattttgtcacgtatatgaaaaacaatggcgttaatctttatgcaatttcagttcaaaatgaaccggattatgcacatgaatggacatggtggacgcctcaagaaattctgagatttatgagagagaacgcaggctcaatcaatgcaagagttattgccccggaatcatttcaatacctgaagaatattagcgacccgattcttaatgatccgcaggcactgagaaatatggatattctgggcgcacacctgtatggcacgcaaatttcacaactgccgtatccgctgttcaagcaaaaaggagcaggcaaagaactgtggatgacagaagtttattacccgaactcagacaacaacagcgcagatagatggcctgaagctctgggagtttcagaacacattcatcactcaatggtcgaaggcgattttcaagcgtacgtttggtggtacattagaagatcatatggcccgatgaaagaggacggcatgatttcaaaaagaggctacaatatggcgcatttttcaaagtttgttagaccgggctatgtcagaatcgacgcgacgaaatcaccggaaccgaacgtttttgtttcagcctataaaggagataacaaggtcgttattgttgccattaacaagaataacacaggcgtcaaccaacactttgttatgcagaatggaacggcatcacaagcaagcagatggattacatcatcaaattcaaatctgcaaccgggcacggaccttaacatctcaggaaaccaattttgggctcatctgcctgcacagagcgttacaacatttgttgttaaaagatga

SEQ ID NO: 22 - protein, 421 aa, artificial sequence
  amino acid sequence of the BsaXyn1 full length recombinant protein expressed from plasmid p2JM (aprE- BsaXyn1)
MRSKKLWISLLFALTLIFTMAFSNMSAQAAGKASDANINVNAERQVIRGFGGMNHPAWIGDLTAPQRETAFGNGQNQLGFSILRINVDENRNNWHREVATAKRAIEHGALVIASPWNPPSHMVETFNRNGASAKRLRYNQYAAYAQHLNDFVTYMKNNGVNLYAISVQNEPDYAHEWTWWTPQEILRFMRENAGSINARVIAPESFQYLKNISDPILNDPQALRNMDILGAHLYGTQISQLPYPLFKQKGAGKELWMTEVYYPNSDNNSADRWPEALGVSEHIHHSMVEGDFQAYVWWYIRRSYGPMKEDGMISKRGYNMAHFSKFVRPGYVRIDATKSPEPNVFVSAYKGDNKVVIVAINKNNTGVNQHFVMQNGTASQASRWITSSNSNLQPGTDLNISGNQFWAHLPAQSVTTFVVKR

SEQ ID NO: 23 - DNA, 1347 bp, artificial sequence
  nucleotide sequence of PmaXyn4 gene from plasmid p2JM (aprE- PmaXyn4)
gtgagaagcaaaaaattgtggatcagcttgttgtttgcgttaacgttaatctttacgatggcgttcagcaacatgagcgcgcaggctgctggaaaagcatcagatgcagttgtcaatgtcagcgcagagaaacaagttattagaggctttggcggcatcaatcatcctgcgtggattggcgaccttacagctgctcagagagagacagcatttggcaatggcaacaatcaactgggctttagcattctgagaatttacgttcatgatgatagaaatcaatggtatagagaactggaaacagccaaaagagctatcgcacttggagcaattgtttttgcatcaccgtggaatccgcctgcagatatggtcgagacatttaacagaaatggcgatacatcagcgaagagactgagatatgacaagtatgcagcgtatgcacaacatctgaacgattttgttacgtatatgagaaataatggagttaatctgtatgcaatttcagttcaaaatgaaccggattatgctcacgattggacatggtggacaccgcaagaaatgcttagatttatgaaggaaaatgcaggatcaattaacgcaagagtgattgcaccggaatcatttcaatatctgaagaatatgtcagatccgattctgaatgactcacaagcactggcaaatatggacattcttggcgcacatctttatggaacacaaatttcaaactttgcatatcctctgttcaaacaaaaaggcgcaggcaaagaactgtggatgacagaagtctattatccgaatagcgacaacaattcagcagatagatggccggaggcactggatgttagctatcatattcataatgccatggttgaaggagacttccaagcatatgtttggtggtatattagaagatcatacggcccgatgaaagaagatggcacgatctcaaagagaggctacaatatggcgcattttagcaagttcgttagaccgggctatgttagagttgacgctacaaaaaatccggaaacaaatgtttatgtttcagcatataaaggcaataacaagatcgtcatcgttgcgattaatagatcaggctcaggcgttaaccagaactttgtcctgagaaacggctcagtttcaaaggttagcagatggattacaaattcatcatcaaatctgcaaccgggcacggaactgacggtcacaggcgaaaatttctgggcacacctgcctgcacagtcagtgacgacgtttgtggcagaccttggcacagcatcaggcagaagcgcagcaaatgaagcagaaacagatacaacgctgccggatgcggttgttgataatctgaga

SEQ ID NO: 24 - protein, 449 aa, artificial sequence
  amino acid sequence of the PmaXyn4 full length recombinant protein expressed from plasmid p2JM (aprE- PmaXyn4)
MRSKKLWISLLFALTLIFTMAFSNMSAQAAGKASDAVVNVSAEKQVIRGFGGINHPAWIGDLTAAQRETAFGNGNNQLGFSILRIYVHDDRNQWYRELETAKRAIALGAIVFASPWNPPADMVETFNRNGDTSAKRLRYDKYAAYAQHLNDFVTYMRNNGVNLYAISVQNEPDYAHDWTWWTPQEMLRFMKENAGSINARVIAPESFQYLKNMSDPILNDSQALANMDILGAHLYGTQISNFAYPLFKQKGAGKELWMTEVYYPNSDNNSADRWPEALDVSYHIHNAMVEGDFQAYVWWYIRRSYGPMKEDGTISKRGYNMAHFSKFVRPGYVRVDATKNPETNVYVSAYKGNNKIVIVAINRSGSGVNQNFVLRNGSVSKVSRWITNSSSNLQPGTELTVTGENFWAHLPAQSVTTFVADLGTASGRSAANEAETDTTLPDAVVDNLR

SEQ ID NO: 25 - DNA, 1659 bp, artificial sequence
  nucleotide sequence of PcoXyn1 gene from plasmid p2JM (aprE- PcoXyn1)
gtgagaagcaaaaaattgtggatcagcttgttgtttgcgttaacgttaatctttacgatggcgttcagcaacatgagcgcgcaggctgctggaaaagcatcagatgcaacaattaatcttgctgctgaaaaacaagtgattagaggctttggcggcattaatcatccggtttgggcaggcgatctgacagctgcacagagagagacagcatttggcaacggcgacaatcaactgggcttttcagttctgagaatccatgttgacgaagatagaaaccaatggagcaaagaagttgagacggcaaaaagcgcaatcgctagaggcgcaattgttttcgcttcaccgtggaatccgccgagcgatatgacggagacatttaacagaaatggcgatacaagcgcgaaaagacttagatacgacaaatatgcagcatacgcacaatatctgaatgactttgttacatacatgaagaacaatggcgtcgatctttatgcaatctcagtccaaaacgaacctgactatgcacatacgtggacatggtggacaccggcggagatgcttagatttatgaaagaaaatgcgggaagcattcaatgtagagttattgcaccggaaagcttctcataccttaaaaacatgagcgatccgattcttaatgatccgcaggcacttgcaaacatggacattctgggcgcacacctgtatggaacaccgtttgacaatttcagctacccgctgtttaaggaaaaaggagcaggcaaagatctgtggatgacagaagtttattacccgaactcagataacaatagcgcagatagatggcctgaagcacttgacgtctcatatcacatccataaagcgatggccaaaggcgattttcaagcatatgtctggtggtatattagaagacaatatggcccgattaaagaggatggatcaattagcaaaagaggctataacatggctcacttttcaaaatttgttagaccgggatatgttagaattgatgctacagagaatcctgatacggatgtttatacgagcgcctataagggcgataataaggttgtcgtcgtggcaatcaatagaggaacatcagcgaaatcacagcactttgtcctgcagaacggaacagcttcaaaagtgtcatcatgggttacagacgcaggaagaaatctggctcctggctcagttcacacgagcggcgattcatttacggcacaactgcctgcacaatcagtcacaacattcgttgttgatctgggatcaaatggcagcacatatgaagcagagtcaggcacgacacttacagatgcggttgtggaaacagtcaatccgggctatcacggaacgggctacgttaactttaatgcaagcagcggcgcagccgttcaatggaatggcatttattgcgcagttgcgggcacgaaaaatgtcgattttagatatgcactggaaagcggctcaagaaaagttgacgtttatgttaatggaacgaaggctatctcaaatgcagagtttacagcgacaggatcatggagcgcatggagaaatcaaacgattcaagtttcaatgaatagcggaattaacacactgaaagttgttacgacgggaacagaaggcccgaatatggatagcgttacagtttcaccgggctca

SEQ ID NO: 26 - protein, 553 aa, artificial sequence
  amino acid sequence of the PcoXyn1 full length recombinant protein expressed from plasmid (aprE- PcoXyn1)
MRSKKLWISLLFALTLIFTMAFSNMSAQAAGKASDATINLAAEKQVIRGFGGINHPVWAGDLTAAQRETAFGNGDNQLGFSVLRIHVDEDRNQWSKEVETAKSAIARGAIVFASPWNPPSDMTETFNRNGDTSAKRLRYDKYAAYAQYLNDFVTYMKNNGVDLYAISVQNEPDYAHTWTWWTPAEMLRFMKENAGSIQCRVIAPESFSYLKNMSDPILNDPQALANMDILGAHLYGTPFDNFSYPLFKEKGAGKDLWMTEVYYPNSDNNSADRWPEALDVSYHIHKAMAKGDFQAYVWWYIRRQYGPIKEDGSISKRGYNMAHFSKFVRPGYVRIDATENPDTDVYTSAYKGDNKVVVVAINRGTSAKSQHFVLQNGTASKVSSWVTDAGRNLAPGSVHTSGDSFTAQLPAQSVTTFVVDLGSNGSTYEAESGTTLTDAVVETVNPGYHGTGYVNFNASSGAAVQWNGIYCAVAGTKNVDFRYALESGSRKVDVYVNGTKAISNAEFTATGSWSAWRNQTIQVSMNSGINTLKVVTTGTEGPNMDSVTVSPGS

SEQ ID NO: 27 - DNA, 1272 bp, artificial sequence
  nucleotide sequence of PtuXyn2 gene from plasmid p2JM (aprE- PtuXyn2)
gtgagaagcaaaaaattgtggatcagcttgttgtttgcgttaacgttaatctttacgatggcgttcagcaacatgagcgcgcaggctgctggaaaagcatcagatgtgacagttaacctgtcatcacaaaaacagcttattaagggctttggaggaattaatcatccggcatggattggcgatcttacacctgctcagagagatacagccttcggcaacggccaaaatcaactgggctttagcattctgagagtgtatatcgatgataataagaacaattggtataaagaagtggctacagccaagagagccattgaacaaggcgccatcgtttttgcatcaccgtggaatccgccgtcagacatggttgaaacgttcaatagaaatggcgatacaacagcaaaaagacttaaatacgataagtatgcagcatatagccaacatcttaatgatttcgtttcatacatgaagtcaaatggagttaatctgtatgcaatttcagtgcaaaatgaaccggattatgcacatgattggacgtggtggacaccgcaagaaatgctgagatttatgaaagattacgctggctcaattacaggcgcaaaagttatggcaccggagtcattttcatatctgaaagagatgtcagatcctattctgaatgatccgcaagccctggcaaatatggatattctgggcgcacatacatacggcacacaatttaacaattttccgtatcctctttttaagcaaaagggcgcaggaaaagagctgtggatgagcgaggtttattacccgaactcaaatgcaaattcagcagataattggccggaagctctggatgttagctaccatattcataatgcaatggtggaagcagattttcaagcatatgtttggtggtatattagaagacaatatggcccgatgaaggaagatggaacgatctcaaagagaggctacaacatggctcatttttcaaagtttgttagaccgggcttcgttagagttgaagcgacaaaaaatcctgatacacagacattcattagcgcatataaaggcgataacaaagtggttatcgttgccattaacagaggaacatcagcggttaatcagaaatttgttctgcaaaacggcaatgcatcaacagttagcagctggattacagactcaacaagaaacctggctgcaggctcatcaatcaatgttacgggcaattcatttacagcacaactgcctgcacagtcagttacaacgtttacggctcctctgaaa

SEQ ID NO: 28 - protein, 424 aa, artificial sequence
  amino acid sequence of the PtuXyn2 precursor protein expressed from plasmid p2JM (aprE- PtuXyn2)
MRSKKLWISLLFALTLIFTMAFSNMSAQAAGKASDVTVNLSSQKQLIKGFGGINHPAWIGDLTPAQRDTAFGNGQNQLGFSILRVYIDDNKNNWYKEVATAKRAIEQGAIVFASPWNPPSDMVETFNRNGDTTAKRLKYDKYAAYSQHLNDFVSYMKSNGVNLYAISVQNEPDYAHDWTWWTPQEMLRFMKDYAGSITGAKVMAPESFSYLKEMSDPILNDPQALANMDILGAHTYGTQFNNFPYPLFKQKGAGKELWMSEVYYPNSNANSADNWPEALDVSYHIHNAMVEADFQAYVWWYIRRQYGPMKEDGTISKRGYNMAHFSKFVRPGFVRVEATKNPDTQTFISAYKGDNKVVIVAINRGTSAVNQKFVLQNGNASTVSSWITDSTRNLAAGSSINVTGNSFTAQLPAQSVTTFTAPLK

SEQ ID NO: 29 - protein, 390 aa, Bacillus subtilis
ASDVTVNVSAEKQVIRGFGGMNHPAWAGDLTAAQRETAFGNGQNQLGFSILRIHVDENRNNWYKEVETAKSAVKHGAIVFASPWNPPSDMVETFNRNGDTSAKRLKYNKYAAYAQHLNDFVTFMKNNGVNLYAISVQNEPDYAHEWTWWTPQEILRFMRENAGSINARVIAPESFQYLKNLSDPILNDPQALANMDILGTHLYGTQVSQFPYPLFKQKGAGKDLWMTEVYYPNSDTNSADRWPEALDVSQHIHNAMVEGDFQAYVWWYIRRSYGPMKEDGTISKRGYNMAHFSKFVRPGYVRIDATKNPNANVYVSAYKGDNKVVIVAINKSNTGVNQNFVLQNGSASNVSRWITSSSSNLQPGTNLTVSGNHFWAHLPAQSVTTFVVNR

SEQ ID NO: 30 - protein, 391 aa, Bacillus licheniformis
ASDATVRLSAEKQVIRGFGGMNHPAWIGDLTAAQRETAFGNGQNQLGFSILRIHVDENRNNWYREVETAKSAIKHGAIVFASPWNPPSDMVETFNRNGDTSAKRLRYDKYAAYAKHLNDFVTFMKNNGVNLYAISVQNEPDYAHDWTWWTPQEILRFMKENAGSINARVIAPESFQYLKNISDPILNDPKALANMDILGAHLYGTQLNNFAYPLFKQKGAGKDLWMTEVYYPNSDNNSADRWPEALDVSHHIHNSMVEGDFQAYVWWYIRRSYGPMKEDGTISKRGYNMAHFSKFVRPGYVRVDATKSPASNVYVSAYKGDNKVVIVAINKNNSGVNQNFVLQNGSVSQVSRWITSSSSNLQPGTNLNVTDNHFWAHLPAQSVTTFVANLR

SEQ ID NO: 31 - protein, 390 aa, Bacillus amyloliquefaciens FZB42
ASDATVNISAERQVIRGFGGMNHPAWIGDLTAAQRETAFGNGQNQLGFSVLRIHVDENRNNWYKEVETAKSAIKHGAIVFASPWNPPNDMVETFNHNGDTTAKRLRYDKYAAYAQHLNDFVNFMKSNGVNLYAISMQNEPDYAHEWTWWTPQEILRFMRENAGSINARVIAPESFQYLKNISDPILNDPQALRNMDILGTHLYGTQVSQFPYPLFKQKGGGKELWMTEVYYPNSDNNSADRWPEALGVSEHIHHSMVEGDFQAYVWWYIRRSYGPMKEDGMISKRGYNMAHFSKFVRPGYVRIDATKNPEPNVYVSAYKGDNKVVIVAINKNNTGVNQNFVLQNGTASQVSRWITSSSSNLQPGTDLKVTDNHFWAHLPAQSVTTFVVKR

SEQ ID NO: 32 - protein, 389 aa, Bacillus safensis
ASDANINVNAERQVIRGFGGMNHPAWIGDLTAPQRETAFGNGQNQLGFSILRINVDENRNNWHREVATAKRAIEHGALVIASPWNPPSHMVETFNRNGASAKRLRYNQYAAYAQHLNDFVTYMKNNGVNLYAISVQNEPDYAHEWTWWTPQEILRFMRENAGSINARVIAPESFQYLKNISDPILNDPQALRNMDILGAHLYGTQISQLPYPLFKQKGAGKELWMTEVYYPNSDNNSADRWPEALGVSEHIHHSMVEGDFQAYVWWYIRRSYGPMKEDGMISKRGYNMAHFSKFVRPGYVRIDATKSPEPNVFVSAYKGDNKVVIVAINKNNTGVNQHFVMQNGTASQASRWITSSNSNLQPGTDLNISGNQFWAHLPAQSVTTFVVKR

SEQ ID NO: 33 - protein, 417 aa, Paenibacillus macerans
ASDAVVNVSAEKQVIRGFGGINHPAWIGDLTAAQRETAFGNGNNQLGFSILRIYVHDDRNQWYRELETAKRAIALGAIVFASPWNPPADMVETFNRNGDTSAKRLRYDKYAAYAQHLNDFVTYMRNNGVNLYAISVQNEPDYAHDWTWWTPQEMLRFMKENAGSINARVIAPESFQYLKNMSDPILNDSQALANMDILGAHLYGTQISNFAYPLFKQKGAGKELWMTEVYYPNSDNNSADRWPEALDVSYHIHNAMVEGDFQAYVWWYIRRSYGPMKEDGTISKRGYNMAHFSKFVRPGYVRVDATKNPETNVYVSAYKGNNKIVIVAINRSGSGVNQNFVLRNGSVSKVSRWITNSSSNLQPGTELTVTGENFWAHLPAQSVTTFVADLGTASGRSAANEAETDTTLPDAVVDNLR

SEQ ID NO: 34 - protein, 521 aa, Paenibacillus cookii DSM 16944
ASDATINLAAEKQVIRGFGGINHPVWAGDLTAAQRETAFGNGDNQLGFSVLRIHVDEDRNQWSKEVETAKSAIARGAIVFASPWNPPSDMTETFNRNGDTSAKRLRYDKYAAYAQYLNDFVTYMKNNGVDLYAISVQNEPDYAHTWTWWTPAEMLRFMKENAGSIQCRVIAPESFSYLKNMSDPILNDPQALANMDILGAHLYGTPFDNFSYPLFKEKGAGKDLWMTEVYYPNSDNNSADRWPEALDVSYHIHKAMAKGDFQAYVWWYIRRQYGPIKEDGSISKRGYNMAHFSKFVRPGYVRIDATENPDTDVYTSAYKGDNKVVVVAINRGTSAKSQHFVLQNGTASKVSSWVTDAGRNLAPGSVHTSGDSFTAQLPAQSVTTFVVDLGSNGSTYEAESGTTLTDAVVETVNPGYHGTGYVNFNASSGAAVQWNGIYCAVAGTKNVDFRYALESGSRKVDVYVNGTKAISNAEFTATGSWSAWRNQTIQVSMNSGINTLKVVTTGTEGPNMDSVTVSPGS

SEQ ID NO: 35 - protein, 392 aa, Paenibacillus tundrae DSM 21291
ASDVTVNLSSQKQLIKGFGGINHPAWIGDLTPAQRDTAFGNGQNQLGFSILRVYIDDNKNNWYKEVATAKRAIEQGAIVFASPWNPPSDMVETFNRNGDTTAKRLKYDKYAAYSQHLNDFVSYMKSNGVNLYAISVQNEPDYAHDWTWWTPQEMLRFMKDYAGSITGAKVMAPESFSYLKEMSDPILNDPQALANMDILGAHTYGTQFNNFPYPLFKQKGAGKELWMSEVYYPNSNANSADNWPEALDVSYHIHNAMVEADFQAYVWWYIRRQYGPMKEDGTISKRGYNMAHFSKFVRPGFVRVEATKNPDTQTFISAYKGDNKVVIVAINRGTSAVNQKFVLQNGNASTVSSWITDSTRNLAAGSSINVTGNSFTAQLPAQSVTTFTAPLK

SEQ ID NO: 36 - protein, 393 aa, artificial sequence
  amino acid sequence of the BsuGH30 mature protein expressed from plasmid p2JM (aprE- BsuGH30)
AGKASDVTVNVSAEKQVIRGFGGMNHPAWAGDLTAAQRETAFGNGQNQLGFSILRIHVDENRNNWYKEVETAKSAVKHGAIVFASPWNPPSDMVETFNRNGDTSAKRLKYNKYAAYAQHLNDFVTFMKNNGVNLYAISVQNEPDYAHEWTWWTPQEILRFMRENAGSINARVIAPESFQYLKNLSDPILNDPQALANMDILGTHLYGTQVSQFPYPLFKQKGAGKDLWMTEVYYPNSDTNSADRWPEALDVSQHIHNAMVEGDFQAYVWWYIRRSYGPMKEDGTISKRGYNMAHFSKFVRPGYVRIDATKNPNANVYVSAYKGDNKVVIVAINKSNTGVNQNFVLQNGSASNVSRWITSSSSNLQPGTNLTVSGNHFWAHLPAQSVTTFVVNR

SEQ ID NO: 37 - protein, 394 aa, artificial sequence
  amino acid sequence of the BliXyn1 mature protein expressed from plasmid p2JM (aprE- BliXyn1)
AGKASDATVRLSAEKQVIRGFGGMNHPAWIGDLTAAQRETAFGNGQNQLGFSILRIHVDENRNNWYREVETAKSAIKHGAIVFASPWNPPSDMVETFNRNGDTSAKRLRYDKYAAYAKHLNDFVTFMKNNGVNLYAISVQNEPDYAHDWTWWTPQEILRFMKENAGSINARVIAPESFQYLKNISDPILNDPKALANMDILGAHLYGTQLNNFAYPLFKQKGAGKDLWMTEVYYPNSDNNSADRWPEALDVSHHIHNSMVEGDFQAYVWWYIRRSYGPMKEDGTISKRGYNMAHFSKFVRPGYVRVDATKSPASNVYVSAYKGDNKVVIVAINKNNSGVNQNFVLQNGSVSQVSRWITSSSSNLQPGTNLNVTDNHFWAHLPAQSVTTFVANLR

SEQ ID NO: 38 - protein, 393 aa, artificial sequence
  amino acid sequence of the BamGh2 mature protein expressed from plasmid p2JM (aprE- BamGh2)
AGKASDATVNISAERQVIRGFGGMNHPAWIGDLTAAQRETAFGNGQNQLGFSVLRIHVDENRNNWYKEVETAKSAIKHGAIVFASPWNPPNDMVETFNHNGDTTAKRLRYDKYAAYAQHLNDFVNFMKSNGVNLYAISMQNEPDYAHEWTWWTPQEILRFMRENAGSINARVIAPESFQYLKNISDPILNDPQALRNMDILGTHLYGTQVSQFPYPLFKQKGGGKELWMTEVYYPNSDNNSADRWPEALGVSEHIHHSMVEGDFQAYVWWYIRRSYGPMKEDGMISKRGYNMAHFSKFVRPGYVRIDATKNPEPNVYVSAYKGDNKVVIVAINKNNTGVNQNFVLQNGTASQVSRWITSSSSNLQPGTDLKVTDNHFWAHLPAQSVTTFVVKR

SEQ ID NO: 39 - protein, 392 aa, artificial sequence
  amino acid sequence of the BsaXyn1 mature protein expressed from plasmid p2JM (aprE-BsaXyn1)
AGKASDANINVNAERQVIRGFGGMNHPAWIGDLTAPQRETAFGNGQNQLGFSILRINVDENRNNWHREVATAKRAIEHGALVIASPWNPPSHMVETFNRNGASAKRLRYNQYAAYAQHLNDFVTYMKNNGVNLYAISVQNEPDYAHEWTWWTPQEILRFMRENAGSINARVIAPESFQYLKNISDPILNDPQALRNMDILGAHLYGTQISQLPYPLFKQKGAGKELWMTEVYYPNSDNNSADRWPEALGVSEHIHHSMVEGDFQAYVWWYIRRSYGPMKEDGMISKRGYNMAHFSKFVRPGYVRIDATKSPEPNVFVSAYKGDNKVVIVAINKNNTGVNQHFVMQNGTASQASRWITSSNSNLQPGTDLNISGNQFWAHLPAQSVTTFVVKR

SEQ ID NO: 40 - protein, 420 aa, artificial sequence
  amino acid sequence of the PmaXyn4 mature protein expressed from plasmid p2JM (aprE- PmaXyn4)
AGKASDAVVNVSAEKQVIRGFGGINHPAWIGDLTAAQRETAFGNGNNQLGFSILRIYVHDDRNQWYRELETAKRAIALGAIVFASPWNPPADMVETFNRNGDTSAKRLRYDKYAAYAQHLNDFVTYMRNNGVNLYAISVQNEPDYAHDWTWWTPQEMLRFMKENAGSINARVIAPESFQYLKNMSDPILNDSQALANMDILGAHLYGTQISNFAYPLFKQKGAGKELWMTEVYYPNSDNNSADRWPEALDVSYHIHNAMVEGDFQAYVWWYIRRSYGPMKEDGTISKRGYNMAHFSKFVRPGYVRVDATKNPETNVYVSAYKGNNKIVIVAINRSGSGVNQNFVLRNGSVSKVSRWITNSSSNLQPGTELTVTGENFWAHLPAQSVTTFVADLGTASGRSAANEAETDTTLPDAVVDNLR

SEQ ID NO: 41 - protein, 524 aa, artificial sequence
  amino acid sequence of the PcoXyn1 mature protein expressed from plasmid p2JM (aprE-PcoXyn1)
AGKASDATINLAAEKQVIRGFGGINHPVWAGDLTAAQRETAFGNGDNQLGFSVLRIHVDEDRNQWSKEVETAKSAIARGAIVFASPWNPPSDMTETFNRNGDTSAKRLRYDKYAAYAQYLNDFVTYMKNNGVDLYAISVQNEPDYAHTWTWWTPAEMLRFMKENAGSIQCRVIAPESFSYLKNMSDPILNDPQALANMDILGAHLYGTPFDNFSYPLFKEKGAGKDLWMTEVYYPNSDNNSADRWPEALDVSYHIHKAMAKGDFQAYVWWYIRRQYGPIKEDGSISKRGYNMAHFSKFVRPGYVRIDATENPDTDVYTSAYKGDNKVVVVAINRGTSAKSQHFVLQNGTASKVSSWVTDAGRNLAPGSVHTSGDSFTAQLPAQSVTTFVVDLGSNGSTYEAESGTTLTDAVVETVNPGYHGTGYVNFNASSGAAVQWNGIYCAVAGTKNVDFRYALESGSRKVDVYVNGTKAISNAEFTATGSWSAWRNQTIQVSMNSGINTLKVVTTGTEGPNMDSVTVSPGS

SEQ ID NO: 42 - protein, 395 aa, artificial sequence
  amino acid sequence of the PtuXyn2 mature protein expressed from plasmid p2JM (aprE- PtuXyn2)
AGKASDVTVNLSSQKQLIKGFGGINHPAWIGDLTPAQRDTAFGNGQNQLGFSILRVYIDDNKNNWYKEVATAKRAIEQGAIVFASPWNPPSDMVETFNRNGDTTAKRLKYDKYAAYSQHLNDFVSYMKSNGVNLYAISVQNEPDYAHDWTWWTPQEMLRFMKDYAGSITGAKVMAPESFSYLKEMSDPILNDPQALANMDILGAHTYGTQFNNFPYPLFKQKGAGKELWMSEVYYPNSNANSADNWPEALDVSYHIHNAMVEADFQAYVWWYIRRQYGPMKEDGTISKRGYNMAHFSKFVRPGFVRVEATKNPDTQTFISAYKGDNKVVIVAINRGTSAVNQKFVLQNGNASTVSSWITDSTRNLAAGSSINVTGNSFTAQLPAQSVTTFTAPLK

SEQ ID NO: 43 - DNA, 607 bp, Bacillus subtilis
aattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaaga

SEQ ID NO: 44 - protein, 29 aa, Bacillus subtilis
MRSKKLWISLLFALTLIFTMAFSNMSAQA

SEQ ID NO: 45 - DNA, 247 bp, Bacillus subtilis
acataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 46 - protein, 328 aa, Fusarium verticilloides
MKLSSFLYTASLVAAIPTAIEPRQAADSINKLIKNKGKLYYGTITDPNLLGVAKDTAIIKADFGAVTPENSGKWDATEPSQGKFNFGSFDQVVNFAQQNGLKVRGHTLVWHSQLPQWVKNINDKATLTKVIENHVTQVVGRYKGKIYAWDVVNEIFEWDGTLRKDSHFNNVFGNDDYVGIAFRAARKADPNAKLYINDYSLDSGSASKVTKGMVPSVKKWLSQGVPVDGIGSQTHLDPGAAGQIQGALTALANSGVKEVAITELDIRTAPANDYATVTKACLNVPKCIGITVWGVSDKNSWRKEHDSLLFDANYNPKPAYTAVVNALR

SEQ ID NO: 47 - protein, 313 aa, Fusarium verticilloides
IPTAIEPRQAADSINKLIKNKGKLYYGTITDPNLLGVAKDTAIIKADFGAVTPENSGKWDATEPSQGKFNFGSFDQVVNFAQQNGLKVRGHTLVWHSQLPQWVKNINDKATLTKVIENHVTQVVGRYKGKIYAWDVVNEIFEWDGTLRKDSHFNNVFGNDDYVGIAFRAARKADPNAKLYINDYSLDSGSASKVTKGMVPSVKKWLSQGVPVDGIGSQTHLDPGAAGQIQGALTALANSGVKEVAITELDIRTAPANDYATVTKACLNVPKCIGITVWGVSDKNSWRKEHDSLLFDANYNPKPAYTAVVNALR

SEQ ID NO: 48 - protein, 305 aa, Fusarium verticilloides
QAADSINKLIKNKGKLYYGTITDPNLLGVAKDTAIIKADFGAVTPENSGKWDATEPSQGKFNFGSFDQVVNFAQQNGLKVRGHTLVWHSQLPQWVKNINDKATLTKVIENHVTQVVGRYKGKIYAWDVVNEIFEWDGTLRKDSHFNNVFGNDDYVGIAFRAARKADPNAKLYINDYSLDSGSASKVTKGMVPSVKKWLSQGVPVDGIGSQTHLDPGAAGQIQGALTALANSGVKEVAITELDIRTAPANDYATVTKACLNVPKCIGITVWGVSDKNSWRKEHDSLLFDANYNPKPAYTAVVNALR

SEQ ID NO: 49 - DNA, 1039 bp, Fusarium verticilloides
atgaagctgtcttctttcctctacaccgcctcgctggtcgcggccattcccaccgccatcgagccccgccaggctgccgacagcatcaacaagctgatcaagaacaagggcaagctctactacggaaccatcaccgaccccaacctgctcggcgtcgcaaaggacaccgccatcatcaaggccgactttggcgccgttacccccgagaactcgggcaagtgggacgccaccgagcccagccagggcaagttcaacttcggtagcttcgaccaggttgtcaactttgcccagcagaatggcctcaaggtccgaggtcacactctggtctggcactctcagctccctcagtgggttaagaacatcaacgacaaggctactctgaccaaggtcattgagaaccacgtcacccaagtcgttggacgctacaagggcaagatctacgcctgggtatgttttattcccccagacttcttcgaaatgactttgctaacatgttcaggacgtcgtcaacgagatcttcgagtgggacggtaccctccgaaaggactctcacttcaacaacgtcttcggcaacgacgactacgttggcattgccttccgcgccgcccgcaaggctgaccccaacgccaagctgtacatcaacgactacagcctcgactccggcagcgcctccaaggtcaccaagggtatggttccctccgtcaagaagtggctcagccagggcgttcccgtcgacggcattggctctcagactcaccttgaccccggtgccgctggccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttacactgctgttgtcaacgctctccgctaa

SEQ ID NO: 50 - DNA, 987 bp, Fusarium verticilloides
atgaagctgtcttctttcctctacaccgcctcgctggtcgcggccattcccaccgccatcgagccccgccaggctgccgacagcatcaacaagctgatcaagaacaagggcaagctctactacggaaccatcaccgaccccaacctgctcggcgtcgcaaaggacaccgccatcatcaaggccgactttggcgccgttacccccgagaactcgggcaagtgggacgccaccgagcccagccagggcaagttcaacttcggtagcttcgaccaggttgtcaactttgcccagcagaatggcctcaaggtccgaggtcacactctggtctggcactctcagctccctcagtgggttaagaacatcaacgacaaggctactctgaccaaggtcattgagaaccacgtcacccaagtcgttggacgctacaagggcaagatctacgcctgggacgtcgtcaacgagatcttcgagtgggacggtaccctccgaaaggactctcacttcaacaacgtcttcggcaacgacgactacgttggcattgccttccgcgccgcccgcaaggctgaccccaacgccaagctgtacatcaacgactacagcctcgactccggcagcgcctccaaggtcaccaagggtatggttccctccgtcaagaagtggctcagccagggcgttcccgtcgacggcattggctctcagactcaccttgaccccggtgccgctggccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttacactgctgttgtcaacgctctccgctaa

SEQ ID NO: 51 - DNA, 942 bp, Fusarium verticilloides
attcccaccgccatcgagccccgccaggctgccgacagcatcaacaagctgatcaagaacaagggcaagctctactacggaaccatcaccgaccccaacctgctcggcgtcgcaaaggacaccgccatcatcaaggccgactttggcgccgttacccccgagaactcgggcaagtgggacgccaccgagcccagccagggcaagttcaacttcggtagcttcgaccaggttgtcaactttgcccagcagaatggcctcaaggtccgaggtcacactctggtctggcactctcagctccctcagtgggttaagaacatcaacgacaaggctactctgaccaaggtcattgagaaccacgtcacccaagtcgttggacgctacaagggcaagatctacgcctgggacgtcgtcaacgagatcttcgagtgggacggtaccctccgaaaggactctcacttcaacaacgtcttcggcaacgacgactacgttggcattgccttccgcgccgcccgcaaggctgaccccaacgccaagctgtacatcaacgactacagcctcgactccggcagcgcctccaaggtcaccaagggtatggttccctccgtcaagaagtggctcagccagggcgttcccgtcgacggcattggctctcagactcaccttgaccccggtgccgctggccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttacactgctgttgtcaacgctctccgctaa

SEQ ID NO: 52 - protein, 305 aa, Fusarium verticilloides
QAADSIDKLIKNKGKLYYGTITDPPLLGVAKDVAIIKADFGAVTPENSGKWDATEPSQGKFNFTSFDQVVNFAQQNGLYVRGHTLVWHGQLPQWVKNINDKATLTKVIENHVTQVVGRYKGKIYAWDVVNEIFEWDGTLRKDSHFNNVFGNDDYVGIAFRAARKADPNAKLYINDYSLDSGSASKVTKGMVPSVKKWLSQGVPVDGIGSQTHLDPGQAGQIQGALTALANSGVKEVAITELDIRTAPANDYATVTKACLNVPKCIGITVWGVSDKNSWRKEHDSLLFDANYNPKPAYYAVVNALR

SEQ ID NO: 53 - DNA, 987 bp, Fusarium verticilloides
atgaagctgtcttctttcctctacaccgcctcgctggtcgcggccattcccaccgccatcgagccccgccaggctgccgacagcatcgacaagctgatcaagaacaagggcaagctctactacggaaccatcaccgacccccccctgctcggcgtcgcaaaggacgtcgccatcatcaaggccgactttggcgccgttacccccgagaactcgggcaagtgggacgccaccgagcccagccagggcaagttcaacttcaccagcttcgaccaggttgtcaactttgcccagcagaatggcctctacgtccgaggtcacactctggtctggcacggccagctccctcagtgggttaagaacatcaacgacaaggctactctgaccaaggtcattgagaaccacgtcacccaagtcgttggacgctacaagggcaagatctacgcctgggacgtcgtcaacgagatcttcgagtgggacggtaccctccgaaaggactctcacttcaacaacgtcttcggcaacgacgactacgttggcattgccttccgcgccgcccgcaaggctgaccccaacgccaagctgtacatcaacgactacagcctcgactccggcagcgcctccaaggtcaccaagggtatggttccctccgtcaagaagtggctcagccagggcgttcccgtcgacggcattggctctcagactcaccttgaccccggtcaggctggccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttactacgctgttgtcaacgctctccgctaa

SEQ ID NO: 54 - DNA, 1039 bp, Fusarium verticilloides
atgaagctgtcttctttcctctacaccgcctcgctggtcgcggccattcccaccgccatcgagccccgccaggctgccgacagcatcgacaagctgatcaagaacaagggcaagctctactacggaaccatcaccgacccccccctgctcggcgtcgcaaaggacgtcgccatcatcaaggccgactttggcgccgttacccccgagaactcgggcaagtgggacgccaccgagcccagccagggcaagttcaacttcaccagcttcgaccaggttgtcaactttgcccagcagaatggcctctacgtccgaggtcacactctggtctggcacggccagctccctcagtgggttaagaacatcaacgacaaggctactctgaccaaggtcattgagaaccacgtcacccaagtcgttggacgctacaagggcaagatctacgcctgggtatgttttattcccccagacttcttcgaaatgactttgctaacatgttcaggacgtcgtcaacgagatcttcgagtgggacggtaccctccgaaaggactctcacttcaacaacgtcttcggcaacgacgactacgttggcattgccttccgcgccgcccgcaaggctgaccccaacgccaagctgtacatcaacgactacagcctcgactccggcagcgcctccaaggtcaccaagggtatggttccctccgtcaagaagtggctcagccagggcgttcccgtcgacggcattggctctcagactcaccttgaccccggtcaggctggccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttactacgctgttgtcaacgctctccgctaa

(19) United States
(12) Patent Application Publication
(10) Pub. No.: US 2021/0277374 A1
(43) Pub. Date: Sep. 9, 2021

(54) XYLANASE-CONTAINING FEED ADDITIVES FOR CEREAL-BASED ANIMAL FEED

(71) Applicant: DUPONT NUTRITION BIOSCIENCES APS, Copenhagen K (DK)

(72) Inventors: Susan Arent LUND, BRABRAND (DK); Marion BERNARDEAU, Caen (FR); Zheyong YU, Shanghai (CN); Zhen QIAN, Palo Alto, CA (US)

(21) Appl. No.: 17/258,247

(22) PCT Filed: Jul. 1, 2019
(86) PCT No.: PCT/US2019/040070

§ 371 Date: Jan. 6, 2021

(30) Foreign Application Priority Data
Jul. 6, 2018 (CN) PCT/CN2018/094752
Jul. 16, 2018 (CN) PCT/CN2018/095761

(51) Int. Cl. C12N 9/24; A23K 10/30; A23K 20/189; A23K 50/30

(52) U.S. Cl. CPC C12N 9/248; C12Y 302/01136; C12N 9/2482; A23K 50/30; A23K 10/30; A23K 20/189; C12Y 302/01008

(57) ABSTRACT

A xylanase-containing feed additive for cereal animal feed is described to facilitate degradation of insoluble glucuronoxylan.

### CROSS-REFERENCE TO RELATED APPLICATIONS

This application claims priority to International Patent Application No. PCT/CN2018/094752, filed Jul. 6, 2018, and International Patent Application No. PCT/CN2018/095761, filed Jul. 16, 2018, the disclosures of each of which are incorporated herein by reference in their entireties.

### INCORPORATION BY REFERENCE OF THE SEQUENCE LISTING

The sequence listing provided in the file named NB40864-WO-PCT[3] Sequence Listing_ST25” with a size of 149 KB which was created on Jun. 26, 2019 and which is filed herewith, is incorporated by reference herein in its entirety.

### FIELD

The field relates to novel xylanases and uses thereof in cereal-based animal feed.

### BACKGROUND

Xylan is a group of hemicelluloses that are found in plant cell walls and some algae. Xylans are polysaccharides made from units of xylose (a pentose sugar). Xylans are almost as ubiquitous as cellulose in plant cell walls and contain predominantly β-linked D-xylose units. The main heteropolymers of hemicellulose are xylan, mannan, galactans and arabinans.

Xylan is also one of the foremost anti-nutritional factors in common use feedstuff raw materials, such as, corn, rice, sorghum, etc.

Corn fiber xylan is complex heteroxylan containing beta-1,4-linked xylose residues. This backbone is highly substituted with monomeric side-chains of arabinose linked to O-2 and/or O-3 of xylose residues, monomeric side-chains of glucuronic acid or its 4-O-methyl derivative and oligomeric side-chains containing arabinose, xylose and sometime galactose residues. Xylan in corn fiber is highly resistant to enzymatic degradation.

Xylanase is the name given to a class of enzymes which degrade the linear polysaccharide beta-1,4-xylan into xylose, thus, breaking down hemicellulose which is one of the major components of plant cell walls. Xylanases are key enzymes for xylan depolymerization and cleave internal glycosidic bonds at random or at specific positions of a xylan backbone into small oligomers. As such, they play a major role in microorganisms thriving on plant sources for the degradation of plant matter into usable nutrients. Xylanases are produced by fungi, bacteria, yeast, marine algae, protozoans, snails, crustaceans, insect, seeds, etc.

Based on structural and genetic information, xylanases have been classified into different Glycoside Hydrolase (GH) families (Henrissat, (1991) Biochem. J. 280, 309-316). The glycosyl hydrolase enzymes, which include xylanases, mannanases, amylases, β-glucanases, cellulases, and other carbohydrases, are classified based on such properties as the sequence of amino acids, their three-dimensional structure and the geometry of their catalytic site (Gilkes, et al., 1991, Microbiol. Reviews 55: 303-315). The enzymes with mainly endo-xylanase activity have been described in GH families, 5, 8, 10, 11, 30 and 98.

As was noted above, xylan in corn fiber and other cereals is highly resistant to enzymatic degradation. Given that corn is used globally in animal feed, there is a need for being able to degrade cereal-derived xylans in order to improve nutrient release.

### SUMMARY

In a first embodiment, there is disclosed an additive for animal feed comprising corn or rice, said feed additive comprising at least one enzyme with glucuronoxylanase activity and at least one enzyme having endo-beta-1,4-xylanase activity wherein degradation of insoluble glucuronoxylan is greater than if either enzyme was used alone.

In another embodiment, the xylanase having glucuronoxylanase activity is a GH30 glucuronoxylanase.

In a second embodiment, the xylanase with glucuronoxylanase activity is derived from Bacillus or Paenibacillus sp.

In another embodiment, the xylanase having glucuronoxylanase activity is derived from B. subtilis or B. licheniformis.

In another embodiment, the xylanase having glucuronoxylanase activity comprises a polypeptide having at least 90% (such as any of 90%, 91%, 92%, 93%, 94%, 95%, 96%, 97%, 98%, 99%, or 100%) sequence identity to a polypeptide selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, and SEQ ID NO:42.

In a third embodiment, the xylanase with endo-beta-1,4-xylanase activity is derived from a filamentous fungus (for example, without limitation, Fusarium sp.).

In another embodiment, the xylanase with endo-beta-1,4-xylanase activity comprises a polypeptide having at least 90% (such as any of 90%, 91%, 92%, 93%, 94%, 95%, 96%, 97%, 98%, 99%, or 100%) sequence identity to a polypeptide selected from the group consisting of SEQ ID NO:46, SEQ ID NO:47, SEQ ID NO:48, and SEQ ID NO:52.

In a fourth embodiment, at least one of the xylanases is recombinantly produced.

In a fifth embodiment, there is disclosed a feed additive comprising at least one enzyme with glucuronoxylanase activity and at least one enzyme having endo-beta-1,4-xylanase activity wherein said combination is better in stimulating growth of beneficial bacteria in a digestive tract of a monogastric animal fed a corn based diet when compared to the use of the xylanase having endo-beta-1,4-xylanase activity alone.

In a sixth embodiment, there is described a feed additive comprising at least one enzyme with glucuronoxylanase activity and at least one enzyme having endo-beta-1,4-xylanase activity wherein said combination is capable of increasing production of at least one short chain fatty acid in a monogastric animal fed a corn based diet when compared to the use of the xylanase having endo-beta-1,4-xylanase activity alone.

In a seventh embodiment, the short chain fatty acid is selected from the group consisting of acetic acid, propionic acid or butyric acid.

In an eighth embodiment, any of the feed additives disclosed here may comprise one or more of the enzymes selected the group consisting of an amylase, protease, endo-glucanase and phytase.

In a ninth embodiment, there is disclosed a premix comprising the feed additive of any claims 1-7 and at least one vitamin and/or mineral.

In a tenth embodiment, there is disclosed a corn or rice-based animal feed comprising at least one enzyme with glucuronoxylanase activity and at least one enzyme having endo-beta-1,4-xylanase activity wherein degradation of insoluble glucuronoxylan is greater than if either enzyme was used alone.

In an eleventh embodiment, there is disclosed a corn-based animal feed comprising at least one enzyme with glucuronoxylanase activity and at least one GH10 enzyme having endo-beta-1,4-xylanase activity wherein said combination is better in stimulating growth of beneficial bacteria in a digestive tract of a monogastric animal when compared to the use of the xylanase having endo-beta-1,4-xylanase activity alone.

In a twelfth embodiment, there is disclosed a corn-based animal feed comprising at least one enzyme with glucuronoxylanase activity and at least one enzyme having endo-beta-1,4-xylanase activity wherein said combination is capable of increasing production of at least one short chain fatty acid in a monogastric animal when compared to the use of the xylanase having endo-beta-1,4-xylanase activity alone.

In a thirteenth embodiment, there is disclosed an animal feed wherein the short chain fatty acid is selected from the group consisting of acetic acid, propionic acid or butyric acid.

In a fourteenth embodiment, there is disclosed any of the animal feeds describe herein which further comprises one or more of the enzymes selected the group consisting of an amylase, protease, endo-glucanase and phytase.

In another embodiment, provided herein is a method for degrading insoluble glucuronoxylan in an animal feed comprising corn or rice comprising contacting the corn or rice with at least one enzyme with glucuronoxylanase activity and at least one enzyme having endo-beta-1,4-xylanase activity.

In another embodiment, provided herein is a method for improving the digestibility of insoluble glucuronoxylan in a corn or rice-based animal feed comprising administering to an animal a corn or rice-based animal feed comprising at least one enzyme with glucuronoxylanase activity and at least one enzyme having endo-beta-1,4-xylanase activity.

In another embodiment, the xylanase having glucuronoxylanase activity is a GH30 glucuronoxylanase.

In another embodiment, the xylanase having glucuronoxylanase activity is derived from Bacillus or Paenibacillus sp.

In another embodiment, the xylanase having glucuronoxylanase activity is derived from B. subtilis or B. licheniformis.

In another embodiment, the xylanase having glucuronoxylanase activity comprises a polypeptide having at least 90% (such as any of 90%, 91%, 92%, 93%, 94%, 95%, 96%, 97%, 98%, 99%, or 100%) sequence identity to a polypeptide selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, and SEQ ID NO:42.

In another embodiment, the xylanase having endo-beta-1,4-xylanase activity is derived from a filamentous fungus.

In another embodiment, the xylanase having endo-beta-1,4-xylanase activity comprises a polypeptide having at least 90% (such as any of 90%, 91%, 92%, 93%, 94%, 95%, 96%, 97%, 98%, 99%, or 100%) sequence identity to a polypeptide selected from the group consisting of SEQ ID NO:46, SEQ ID NO:47, SEQ ID NO:48, and SEQ ID NO:52.

In another embodiment, at least one of the xylanases is recombinantly produced.

In another embodiment, the method further comprises administering to the animal (a) one or more of the enzymes selected the group consisting of an amylase, protease, endo-glucanase and phytase; (b) one or more direct fed microbials; or (c) a combination of (a) and (b).

In another embodiment, the animal is a monogastric animal selected from the group consisting of pigs and swine, turkeys, ducks, chicken, salmon, trout, tilapia, catfish, carp, shrimps and prawns.

In another embodiment, the animal is a ruminant animal selected from the group consisting of cattle, young calves, goats, sheep, giraffes, bison, moose, elk, yaks, water buffalo, deer, camels, alpacas, llamas, antelope, pronghorn and nilgai.

### BRIEF DESCRIPTION OF THE DRAWINGS AND SEQUENCES

FIGS. 1A and 1B depict xylanase activity measurement for FveXyn4.v1, BsuGH30 and BliXyn1 enzymes. FIG. 1A depicts the activity dose response of FveXyn4.v1 in the concentration range of 0 to 0.0008 mg/mL, while the responses of BsuGH30 and BliXyn1 were determined in the concentration range of 0 to 0.008 mg/mL. FIG. 1B depicts the activity dose-response curves for BsuGH30 and BliXyn1 within the 0 to 0.004 mg/mL range are linear.

FIG. 2 shows an increase in extractable arabinoxylan reported in xylose equivalents after 2 h incubation of corn DDGS with increasing concentrations of BsuGH30, BliXyn1, FveXyn4 and FveXyn4.v1 enzymes.

FIG. 3 shows an increase in extractable arabinoxylan reported in xylose equivalents after 2 h incubation of corn DDGS with 12.6 μg/g of FveXyn4, FveXyn4.v1 and GH30 glucuronoxylanases (BsuGH30, BliXyn1, BamGh2, BsaXyn1, PmaXyn4, PcoXyn1 and PtuXyn2).

FIG. 4 shows an increase in extractable arabinoxylan reported in xylose equivalents after 2 h incubation of corn DDGS with selected enzymes. FIG. 4A shows a comparison of treatment with 3.2 μg/g GH30 enzymes alone and in combination with 3.2 μg/g FveXyn4. The additive response calculated as the sum of the increase in extractable arabinoxylan obtained from independent treatments with 3.2 μg/g GH30 enzyme and 3.2 μg/g FveXyn4 is also shown. FIG. 4B shows a comparison of treatment with 3.2 μg/g GH30 enzymes alone and in combination with 3.2 μg/g FveXyn4.v1. Also shown is the additive response calculated as the sum of the increase in extractable arabinoxylan obtained from independent treatments with 3.2 μg/g GH30 enzyme and 3.2 μg/g FveXyn4.v1.

FIG. 5 shows an increase in extractable arabinoxylan reported in xylose equivalents. 5A) after 2 h incubation of 5% rice bran with BsuGH30 (GH30 enzyme) and FveXyn4 (GH10 enzyme) either alone or in combination and 5B) after 2 h incubation of 10% rice bran with BliXyn1 and FveXyn4.v1 enzymes either alone or in combination. For the combinations, the xylanase inclusion is the sum of the GH30 enzyme concentration and the GH10 enzyme concentration. The concentration of the GH30 enzyme is stated in the legend box and the concentration of the GH10 enzyme is the difference between the xylanase inclusion on the X-axis and the GH30 enzyme concentration given in the legend box.

FIG. 6 shows an increase in extractable arabinoxylan reported in xylose equivalents after 2 h incubation of corn DDGS with 1.1 μg/g of pretreated enzyme BsuGH30 and BliXyn1. Light grey bars show the control samples, incubated at pH 5.0, and the dark gray bars show results for enzymes pre-incubated with pepsin at pH 3.5.

FIG. 7 sets forth a multiple sequence alignment of full length sequences of GH30 glucuronoxylanases.

The following sequences comply with 37 C.F.R. §§ 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the European Patent Convention (EPC) and the Patent Cooperation Treaty (PCT) Rules 5.2 and 49.5(a-bis), and Section 208 and Annex C of the Administrative Instructions. The symbols and format used for nucleotide and amino acid sequence data comply with the rules set forth in 37 C.F.R. § 1.822.

TABLE 1
Summary of Nucleotide and Amino Acid SEQ ID Numbers
A. GH30 glucuronoxylanases

| | | | Full length | Mature |
|---|---|---|---|---|
| Origin | Name | Gene | Protein | Protein |
| Bacillus subtilis | BsuGH30 | SEQ ID No. 1 | SEQ ID No. 2 | SEQ ID No. 29 |
| Bacillus licheniformis | BliXyn1 | SEQ ID No. 3 | SEQ ID No. 4 | SEQ ID No. 30 |
| Bacillus amyloliquefaciens FZB42 | BamGh2 | SEQ ID No. 5 | SEQ ID No. 6 | SEQ ID No. 31 |
| Bacillus safensis | BsaXyn1 | SEQ ID No. 7 | SEQ ID No. 8 | SEQ ID No. 32 |
| Paenibacillus macerans | PmaXyn4 | SEQ ID No. 9 | SEQ ID No. 10 | SEQ ID No. 33 |
| Paenibacillus cookii DSM 16944 | PcoXyn1 | SEQ ID No. 11 | SEQ ID No. 12 | SEQ ID No. 34 |
| Paenibacillus tundrae DSM 21291 | PtuXyn2 | SEQ ID No. 13 | SEQ ID No. 14 | SEQ ID No. 35 |

B. GH30 glucuronoxylanases (synthetic genes and recombinan protein sequences)

| | | Full length | Mature |
|---|---|---|---|
| | Synthetic | Recombinant | Recombinant |
| Origin | Gene | Protein | Protein |
| Bacillus subtilis | SEQ ID No. 15 | SEQ ID No. 16 | SEQ ID No. 36 |
| Bacillus licheniformis | SEQ ID No. 17 | SEQ ID No. 18 | SEQ ID No. 37 |
| Bacillus amyloliquefaciens FZB42 | SEQ ID No. 19 | SEQ ID No. 20 | SEQ ID No. 38 |
| Bacillus safensis | SEQ ID No. 21 | SEQ ID No. 22 | SEQ ID No. 39 |
| Paenibacillus macerans | SEQ ID No. 23 | SEQ ID No. 24 | SEQ ID No. 40 |
| Paenibacillus cookii DSM 16944 | SEQ ID No. 25 | SEQ ID No. 26 | SEQ ID No. 41 |
| Paenibacillus tundrae DSM 21291 | SEQ ID No. 27 | SEQ ID No. 28 | SEQ ID No. 42 |

### DETAILED DESCRIPTION

All patents, patent applications, and publications cited are incorporated herein by reference in their entirety.

In this disclosure, a number of terms and abbreviations are used. The following definitions apply unless specifically stated otherwise.

The articles “a”, “an”, and “the” preceding an element or component are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

The term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

As used herein in connection with a numerical value, the term “about” refers to a range of +/−0.5 of the numerical value, unless the term is otherwise specifically defined in context. For instance, the phrase a “pH value of about 6” refers to pH values of from 5.5 to 6.5, unless the pH value is specifically defined otherwise.

It is intended that every maximum numerical limitation given throughout this Specification includes every lower numerical limitation, as if such lower numerical limitations were expressly written herein. Every minimum numerical limitation given throughout this Specification will include every higher numerical limitation, as if such higher numerical limitations were expressly written herein. Every numerical range given throughout this Specification will include every narrower numerical range that falls within such broader numerical range, as if such narrower numerical ranges were all expressly written herein.

The term “xylanase” (EC 3.2.1.8, endo-(1->4)-beta-xylan 4-xylanohydrolase, endo-1,4-xylanase, endo-1,4-beta-xylanase, beta-1,4-xylanase, endo-1,4-beta-D-xylanase, 1,4-beta-xylan xylanohydrolase, beta-xylanase, beta-1,4-xylan xylanohydrolase, beta-D-xylanase) means a protein or polypeptide domain derived from a microorganism, e.g. fungi, bacteria, yeast, marine algae, or protozoans. Xylanase has the ability to hydrolyze xylan. The terms “xylanase”, “glycoside hydrolase” and “hydrolase” can be used interchangeably herein.

The term “glucuronoxylanase” (EC 3.2.1.136, glucuronoarabinoxylan endo-1,4-β-xylanase, feraxan endoxylanase, feraxanase, endoarabinoxylanase, glucuronoxylan xylohydrolase, glucuronoxylan xylanohydrolase, glucuronoarabinoxylan 1,4-β-D-xylanohydrolase, glucuronoarabinoxylan 4-β-D-xylanohydrolase) means a protein or polypeptide domain derived from a microorganism, e.g. fungi, bacteria, yeast, marine algae, or protozoans. Glucuronoxylanase has the ability to hydrolyze glucuronoxylan.

The term “glycoside hydrolase” (GH) refers to enzymes that assist in the hydrolysis of the glycosidic linkage of glycosides, i.e., assist in the hydrolysis of glycosidic bonds in complex sugars. Glycoside hydrolases (also called glycosidases or glycosyl hydrolases) assist in the hydrolysis of glycosidic bonds in complex sugars

Glycoside hydrolases (O-Glycosyl hydrolases) EC 3.2.1. are a widespread group of enzymes that hydrolyze the glycosidic bond between two or more carbohydrates, or between a carbohydrate and a non-carbohydrate moiety. A classification system for glycosyl hydrolases, based on sequence similarity, has led to the definition of numerous different families. This classification is available on the CAZy (CArbohydrate-Active EnZymes) web site. Because the fold of proteins is better conserved than their sequences, some of the families can be grouped in ‘clans’. As of October 2011, CAZy includes 128 families of glycosyl hydrolases and 14 clans.

The glycoside hydrolase family 30 (GH30) CAZY GH_30 comprises enzymes with a number of known activities: glucuronoxylanase (EC 3.2.1.136), xylanase (EC 3.2.1.8), β-glucosidase (3.2.1.21), β-glucuronidase (EC 3.2.1.31), β-xylosidase (EC 3.2.1.37), β-fucosidase (EC 3.2.1.38); glucosylceramidase (EC 3.2.1.45), β-1,6-glucanase (EC 3.2.1.75), endo-β-1,6-galactanase (EC:3.2.1.164), and [reducing end] β-xylosidase (EC 3.2.1.-).

Glycoside hydrolase family 10 (GH10) CAZY GH_10 comprises enzymes with a number of known activities: xylanase (EC 3.2.1.8), endo-1,3-beta-xylanase (EC 3.2.1.32), and cellobiohydrolase (EC 3.2.1.91). These enzymes were formerly known as cellulase family F. The microbial degradation of cellulose and xylans requires several types of enzymes such as endoglucanases (EC 3.2.1.4), cellobiohydrolases (EC 3.2.1.91) (exoglucanases), or xylanases (EC 3.2.1.8). Fungi and bacteria produces a spectrum of cellulolytic enzymes (cellulases) and xylanases which, on the basis of sequence similarities, can be classified into families. One of these families is known as the cellulase family F or as the glycosyl hydrolases family.

Glycoside hydrolase family 11 (GH11) CAZY GH_11 comprises enzymes with only two known activities: xylanase (EC 3.2.1.8) and endo-β-1,3-xylanase (EC 3.2.1.32). These enzymes were formerly known as cellulase family G.

The terms “animal” and “subject” are used interchangeably herein. An animal includes all non-ruminant (including humans) and ruminant animals. In a particular embodiment, the animal is a non-ruminant animal, such as a horse and a mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment the animal is a ruminant animal including, but not limited to, cattle, young calves, goats, sheep, giraffes, bison, moose, elk, yaks, water buffalo, deer, camels, alpacas, llamas, antelope, pronghorn and nilgai.

A “feed” means any natural or artificial diet, meal or the like or components of such meals intended or suitable for being eaten, taken in, digested, by a non-human animal and a human being, respectively. The term “feed” is used with reference to products that are fed to animals in the rearing of livestock. The terms “feed” and “animal feed” are used interchangeably.

The term “direct-fed microbial” (“DFM”) as used herein is source of live (viable) naturally occurring microorganisms. A DFM can comprise one or more of such naturally occurring microorganisms such as bacterial strains. Categories of DFMs include Bacillus, Lactic Acid Bacteria and Yeasts. Thus, the term DFM encompasses one or more of the following: direct fed bacteria, direct fed yeast, direct fed yeast and combinations thereof.

Bacilli are unique, gram-positive rods that form spores. These spores are very stable and can withstand environmental conditions such as heat, moisture and a range of pH. These spores germinate into active vegetative cells when ingested by an animal and can be used in meal and pelleted diets. Lactic Acid Bacteria are gram-positive cocci that produce lactic acid which are antagonistic to pathogens. Since Lactic Acid Bacteria appear to be somewhat heat-sensitive, they are not used in pelleted diets. Types of Lactic Acid Bacteria include Bifidobacterium, Lactobacillus and Streptococcus.

The term “prebiotic” means a non-digestible food ingredient that beneficially affects the host by selectively stimulating the growth and/or the activity of one or a limited number of beneficial bacteria.

The term “probiotic culture” as used herein defines live microorganisms (including bacteria or yeasts for example) which, when for example ingested or locally applied in sufficient numbers, beneficially affects the host organism, i.e. by conferring one or more demonstrable health benefits on the host organism. Probiotics may improve the microbial balance in one or more mucosal surfaces. For example, the mucosal surface may be the intestine, the urinary tract, the respiratory tract or the skin. The term “probiotic” as used herein also encompasses live microorganisms that can stimulate the beneficial branches of the immune system and at the same time decrease the inflammatory reactions in a mucosal surface, for example the gut. Whilst there are no lower or upper limits for probiotic intake, it has been suggested that at least 10⁶-10¹², preferably at least 10⁶-10¹⁰, preferably 10⁸-10⁹, cfu as a daily dose will be effective to achieve the beneficial health effects in a subject.

The term “CFU” as used herein means “colony forming units” and is a measure of viable cells in which a colony represents an aggregate of cells derived from a single progenitor cell.

The term “isolated” means a substance in a form or environment that does not occur in nature. Non-limiting examples of isolated substances include (1) any non-naturally occurring substance, (2) any substance including, but not limited to, any host cell, enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated. The terms “isolated nucleic acid molecule”, “isolated polynucleotide”, and “isolated nucleic acid fragment” will be used interchangeably and refer to a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

The term “purified” as applied to nucleic acids or polypeptides generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

As used herein, the term “functional assay” refers to an assay that provides an indication of a protein's activity. In some embodiments, the term refers to assay systems in which a protein is analyzed for its ability to function in its usual capacity. For example, in the case of a xylanase, a functional assay involves determining the effectiveness of the xylanase to hydrolyze xylan.

The terms “peptides”, “proteins” and “polypeptides are used interchangeably herein and refer to a polymer of amino acids joined together by peptide bonds. A “protein” or “polypeptide” comprises a polymeric sequence of amino acid residues. The single and 3-letter code for amino acids as defined in conformity with the IUPAC-IUB Joint Commission on Biochemical Nomenclature (JCBN) is used throughout this disclosure. The single letter X refers to any of the twenty amino acids. It is also understood that a polypeptide may be coded for by more than one nucleotide sequence due to the degeneracy of the genetic code. Mutations can be named by the one letter code for the parent amino acid, followed by a position number and then the one letter code for the variant amino acid. For example, mutating glycine (G) at position 87 to serine (S) is represented as “G087S” or “G87S”. When describing modifications, a position followed by amino acids listed in parentheses indicates a list of substitutions at that position by any of the listed amino acids. For example, 6(L,I) means position 6 can be substituted with a leucine or isoleucine. At times, in a sequence, a slash (/) is used to define substitutions, e.g. F/V, indicates that the particular position may have a phenylalanine or valine at that position.

A “prosequence” or “propeptide sequence” refers to an amino acid sequence between the signal peptide sequence and mature xylanase sequence that is necessary for the proper folding and secretion of the xylanase; they are sometimes referred to as intramolecular chaperones. Cleavage of the prosequence or propeptide sequence results in a mature active xylanase. Xylanase can be expressed as pro-enzymes.

The terms “signal sequence” and “signal peptide” refer to a sequence of amino acid residues that may participate in the secretion or direct transport of the mature or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “short chain fatty acid” also referred to as volatile fatty acids (“VFAs”) are fatty acids with two to six carbon atoms. Short chain fatty acids are produced when dietary fiber is fermented in the colon.

The term “mature” form of a protein, polypeptide, or peptide refers to the functional form of the protein, polypeptide, or enzyme without the signal peptide sequence and propeptide sequence.

The term “precursor” form of a protein or peptide refers to an immature form of the protein having a prosequence operably linked to the amino or carbonyl terminus of the protein. The precursor may also have a “signal” sequence operably linked to the amino terminus of the prosequence. The precursor may also have additional polypeptides that are involved in post-translational activity (e.g., polypeptides cleaved therefrom to leave the mature form of a protein or peptide).

The term “wild-type” in reference to an amino acid sequence or nucleic acid sequence indicates that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

As used herein with regard to amino acid residue positions, “corresponding to” or “corresponds to” or “corresponds” refers to an amino acid residue at the enumerated position in a protein or peptide, or an amino acid residue that is analogous, homologous, or equivalent to an enumerated residue in a protein or peptide. As used herein, “corresponding region” generally refers to an analogous position in a related protein or a reference protein.

The terms “derived from” and “obtained from” refer to not only a protein produced or producible by a strain of the organism in question, but also a protein encoded by a DNA sequence isolated from such strain and produced in a host organism containing such DNA sequence. Additionally, the term refers to a protein which is encoded by a DNA sequence of synthetic and/or cDNA origin and which has the identifying characteristics of the protein in question.

The term “amino acid” refers to the basic chemical structural unit of a protein or polypeptide. The following abbreviations used herein to identify specific amino acids can be found in Table 2.

TABLE 2
One and Three Letter Amino Acid Abbreviations

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Thermostable serine acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid or as defined herein | Xaa | X |

It would be recognized by one of ordinary skill in the art that modifications of amino acid sequences disclosed herein can be made while retaining the function associated with the disclosed amino acid sequences. For example, it is well known in the art that alterations in a gene which result in the production of a chemically equivalent amino acid at a given site, but do not affect the functional properties of the encoded protein are common. For example, any particular amino acid in an amino acid sequence disclosed herein may be substituted for another functionally equivalent amino acid. For the purposes of this disclosure, substitutions are defined as exchanges within one of the following five groups:

1. Small aliphatic, nonpolar or slightly polar residues: Ala, Ser, Thr (Pro, Gly);

2. Polar, negatively charged residues and their amides: Asp, Asn, Glu, Gln;

3. Polar, positively charged residues: His, Arg, Lys;

4. Large aliphatic, nonpolar residues: Met, Leu, Ile, Val (Cys); and

5. Large aromatic residues: Phe, Tyr, and Trp.

Thus, a codon for the amino acid alanine, a hydrophobic amino acid, may be substituted by a codon encoding another less hydrophobic residue (such as glycine) or a more hydrophobic residue (such as valine, leucine, or isoleucine). Similarly, changes which result in substitution of one negatively charged residue for another or one positively charged residue for another (such as lysine for arginine) can also be expected to produce a functionally equivalent product. In many cases, nucleotide changes which result in alteration of the N-terminal and C-terminal portions of the protein molecule would also not be expected to alter the activity of the protein. Each of the proposed modifications is well within the routine skill in the art, as is determination of retention of biological activity of the encoded products.

The term “codon optimized”, as it refers to genes or coding regions of nucleic acid molecules for transformation of various hosts, refers to the alteration of codons in the gene or coding regions of the nucleic acid molecules to reflect the typical codon usage of the host organism without altering the polypeptide for which the DNA codes.

The term “gene” refers to a nucleic acid molecule that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different from that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The term “coding sequence” refers to a nucleotide sequence which codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding sites, and stem-loop structures.

The term “operably linked” refers to the association of nucleic acid sequences on a single nucleic acid molecule so that the function of one is affected by the other. For example, a promoter is operably linked with a coding sequence when it is capable of affecting the expression of that coding sequence, i.e., the coding sequence is under the transcriptional control of the promoter. Coding sequences can be operably linked to regulatory sequences in sense or antisense orientation.

The terms “regulatory sequence” or “control sequence” are used interchangeably herein and refer to a segment of a nucleotide sequence which is capable of increasing or decreasing expression of specific genes within an organism. Examples of regulatory sequences include, but are not limited to, promoters, signal sequence, operators and the like. As noted above, regulatory sequences can be operably linked in sense or antisense orientation to the coding sequence/gene of interest.

“Promoter” or “promoter sequences” refer to DNA sequences that define where transcription of a gene by RNA polymerase begins. Promoter sequences are typically located directly upstream or at the 5′ end of the transcription initiation site. Promoters may be derived in their entirety from a native or naturally occurring sequence, or be composed of different elements derived from different promoters found in nature, or even comprise synthetic DNA segments. It is understood by those skilled in the art that different promoters may direct the expression of a gene in different tissues or cell type or at different stages of development, or in response to different environmental or physiological conditions (“inducible promoters”).

The “3′ non-coding sequences” refer to DNA sequences located downstream of a coding sequence and include sequences encoding regulatory signals capable of affecting mRNA processing or gene expression, such as termination of transcription.

The term “transformation” as used herein refers to the transfer or introduction of a nucleic acid molecule into a host organism. The nucleic acid molecule may be introduced as a linear or circular form of DNA. The nucleic acid molecule may be a plasmid that replicates autonomously, or it may integrate into the genome of a production host. Production hosts containing the transformed nucleic acid are referred to as “transformed” or “recombinant” or “transgenic” organisms or “transformants”.

The terms “recombinant” and “genetically engineered” are used interchangeably herein and refer to an artificial combination of two otherwise separated segments of nucleic acid sequences, e.g., by chemical synthesis or by the manipulation of isolated segments of nucleic acids by genetic engineering techniques. For example, DNA in which one or more segments or genes have been inserted, either naturally or by laboratory manipulation, from a different molecule, from another part of the same molecule, or an artificial sequence, resulting in the introduction of a new sequence in a gene and subsequently in an organism. The terms “recombinant”, “transgenic”, “transformed”, “engineered”, “genetically engineered” and “modified for exogenous gene expression” are used interchangeably herein.

The terms “recombinant construct”, “expression construct”, “recombinant expression construct” and “expression cassette” are used interchangeably herein. A recombinant construct comprises an artificial combination of nucleic acid fragments, e.g., regulatory and coding sequences that are not all found together in nature. For example, a construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:2411-2418; De Almeida et al., (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The terms “production host”, “host” and “host cell” are used interchangeably herein and refer to any organism, or cell thereof, whether human or non-human into which a recombinant construct can be stably or transiently introduced in order to express a gene. This term encompasses any progeny of a parent cell, which is not identical to the parent cell due to mutations that occur during propagation.

The term “percent identity” is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the number of matching nucleotides or amino acids between strings of such sequences. “Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Methods to determine identity and similarity are codified in publicly available computer programs.

As used herein, “% identity” or percent identity” or “PID” refers to protein sequence identity. Percent identity may be determined using standard techniques known in the art. Useful algorithms include the BLAST algorithms (See, Altschul et al., J Mol Biol, 215:403-410, 1990; and Karlin and Altschul, Proc Natl Acad Sci USA, 90:5873-5787, 1993). The BLAST program uses several search parameters, most of which are set to the default values. The NCBI BLAST algorithm finds the most relevant sequences in terms of biological similarity but is not recommended for query sequences of less than 20 residues (Altschul et al., Nucleic Acids Res, 25:3389-3402, 1997; and Schaffer et al., Nucleic Acids Res, 29:2994-3005, 2001). Exemplary default BLAST parameters for a nucleic acid sequence searches include: Neighboring words threshold=11; E-value cutoff=10; Scoring Matrix=NUC.3.1 (match=1, mismatch=−3); Gap Opening=5; and Gap Extension=2. Exemplary default BLAST parameters for amino acid sequence searches include: Word size=3; E-value cutoff=10; Scoring Matrix=BLOSUM62; Gap Opening=11; and Gap extension=1. A percent (%) amino acid sequence identity value is determined by the number of matching identical residues divided by the total number of residues of the “reference” sequence. BLAST algorithms refer to the “reference” sequence as the “query” sequence.

As used herein, “homologous proteins” or “homologous xylanases” refers to proteins that have distinct similarity in primary, secondary, and/or tertiary structure. Protein homology can refer to the similarity in linear amino acid sequence when proteins are aligned. Homologous search of protein sequences can be done using BLASTP and PSI-BLAST from NCBI BLAST with threshold (E-value cut-off) at 0.001. (Altschul S F, Madde T L, Shaffer A A, Zhang J, Zhang Z, Miller W, Lipman D J. Gapped BLAST and PSI BLAST a new generation of protein database search programs. Nucleic Acids Res 1997 Set 1; 25(17):3389-402). Using this information, proteins sequences can be grouped. A phylogenetic tree can be built using the amino acid sequences.

Sequence alignments and percent identity calculations may be performed using the Megalign program of the LASERGENE bioinformatics computing suite (DNASTAR Inc., Madison, Wis.), the AlignX program of Vector NTI v. 7.0 (Informax, Inc., Bethesda, Md.), or the EMBOSS Open Software Suite (EMBL-EBI; Rice et al., Trends in Genetics 16, (6):276-277 (2000)). Multiple alignment of the sequences can be performed using the CLUSTAL method (such as CLUSTALW; for example version 1.83) of alignment (Higgins and Sharp, CABIOS, 5:151-153 (1989); Higgins et al., Nucleic Acids Res. 22:4673-4680 (1994); and Chenna et al., Nucleic Acids Res 31 (13):3497-500 (2003)), available from the European Molecular Biology Laboratory via the European Bioinformatics Institute) with the default parameters. Suitable parameters for CLUSTALW protein alignments include GAP Existence penalty=15, GAP extension=0.2, matrix=Gonnet (e.g., Gonnet250), protein ENDGAP=−1, protein GAPDIST=4, and KTUPLE=1. In one embodiment, a fast or slow alignment is used with the default settings where a slow alignment. Alternatively, the parameters using the CLUSTALW method (e.g., version 1.83) may be modified to also use KTUPLE=1, GAP PENALTY=10, GAP extension=1, matrix=BLOSUM (e.g., BLOSUM64), WINDOW=5, and TOP DIAGONALS SAVED=5.

The MUSCLE program (Robert C. Edgar. MUSCLE: multiple sequence alignment with high accuracy and high throughput Nucl. Acids Res. (2004) 32 (5): 1792-1797) is yet another example of a multiple sequence alignment algorithm.

The term “variant”, with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context. A variant polypeptide sequence or polynucleotide sequence can have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity with a sequence disclosed herein. The variant amino acid sequence or polynucleotide sequence has the same function of the disclosed sequence, or at least about 85%, 86%, 87%, 88%, 89%, 90%, 91%, 92%, 93%, 94%, 95%, 96%, 97%, 98%, or 99% of the function of the disclosed sequence.

The terms “plasmid”, “vector” and “cassette” refer to an extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector containing a gene and having elements in addition to the gene that facilitates transformation of a particular host cell. The terms “expression cassette” and “expression vector” are used interchangeably herein and refer to a specific vector containing a gene and having elements in addition to the gene that allow for expression of that gene in a host.

The term “expression”, as used herein, refers to the production of a functional end-product (e.g., an mRNA or a protein) in either precursor or mature form. Expression may also refer to translation of mRNA into a polypeptide.

Expression of a gene involves transcription of the gene and translation of the mRNA into a precursor or mature protein. “Mature” protein refers to a post-translationally processed polypeptide; i.e., one from which any signal sequence, pre- or propeptides present in the primary translation product have been removed. “Precursor” protein refers to the primary product of translation of mRNA; i.e., with pre- and propeptides still present. Pre- and propeptides may be but are not limited to intracellular localization signals. “Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance.

The expression vector can be one of any number of vectors or cassettes useful for the transformation of suitable production hosts known in the art. Typically, the vector or cassette will include sequences directing transcription and translation of the relevant gene, a selectable marker, and sequences allowing autonomous replication or chromosomal integration. Suitable vectors generally include a region 5′ of the gene which harbors transcriptional initiation controls and a region 3′ of the DNA fragment which controls transcriptional termination. Both control regions can be derived from homologous genes to genes of a transformed production host cell and/or genes native to the production host, although such control regions need not be so derived.

Possible initiation control regions or promoters that can be included in the expression vector are numerous and familiar to those skilled in the art. Virtually any promoter capable of driving these genes is suitable, including but not limited to, CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, araB, tet, trp, IP_{L}, IP_{R}, T7, tac, and trc (useful for expression in Escherichia coli) as well as the amy, apr, npr promoters and various phage promoters useful for expression in Bacillus. In some embodiments, the promoter is a constitutive or inducible promoter. A “constitutive promoter” is a promoter that is active under most environmental and developmental conditions. An “inducible” or “repressible” promoter is a promoter that is active under environmental or developmental regulation. In some embodiments, promoters are inducible or repressible due to changes in environmental factors including but not limited to, carbon, nitrogen or other nutrient availability, temperature, pH, osmolarity, the presence of heavy metal(s), the concentration of inhibitor(s), stress, or a combination of the foregoing, as is known in the art. In some embodiments, the inducible or repressible promoters are inducible or repressible by metabolic factors, such as the level of certain carbon sources, the level of certain energy sources, the level of certain catabolites, or a combination of the foregoing as is known in the art. In one embodiment, the promoter is one that is native to the host cell. For example, in some instances when Trichoderma reesei is the host, the promoter can be a native T. reesei promoter such as the cbh1 promoter which is deposited in GenBank under Accession Number D86235. Other suitable non-limiting examples of promoters useful for fungal expression include, cbh2, egl1, egl2, egl3, egl4, egl5, xyn1, and xyn2, repressible acid phosphatase gene (phoA) promoter of P. chrysogenus (see e.g., Graessle et al., (1997) Appl. Environ. Microbiol., 63:753-756), glucose repressible PCK1 promoter (see e.g., Leuker et al., (1997), Gene, 192:235-240), maltose inducible, glucose-repressible MET3 promoter (see Liu et al., (2006), Eukary. Cell, 5:638-649), pKi promoter and cpc1 promoter. Other examples of useful promoters include promoters from A. awamori and A. niger glucoamylase genes (see e.g., Nunberg et al., (1984) Mol. Cell Biol. 15 4:2306-2315 and Boel et al., (1984) EMBO J. 3:1581-1585). Also, the promoters of the T. reesei xln1 gene may be useful (see e.g., EPA 137280AI).

DNA fragments which control transcriptional termination may also be derived from various genes native to a preferred production host cell. In certain embodiments, the inclusion of a termination control region is optional. In certain embodiments, the expression vector includes a termination control region derived from the preferred host cell.

The expression vector can be included in the production host, particularly in the cells of microbial production hosts. The production host cells can be microbial hosts found within the fungal or bacterial families and which grow over a wide range of temperature, pH values, and solvent tolerances. For example, it is contemplated that any of bacteria, algae, and fungi such as filamentous fungi and yeast may suitably host the expression vector.

Inclusion of the expression vector in the production host cell may be used to express the protein of interest so that it may reside intracellularly, extracellularly, or a combination of both inside and outside the cell. Extracellular expression renders recovery of the desired protein from a fermentation product more facile than methods for recovery of protein produced by intracellular expression.

It is possible to optionally recover the desired protein from the production host. In another aspect, a xylanase-containing culture supernatant is obtained by using any of the methods known to those skilled in the art.

An enzyme secreted from the host cells can be used in a whole broth preparation. The preparation of a spent whole fermentation broth of a recombinant microorganism can be achieved using any cultivation method known in the art resulting in the expression of a xylanase. The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

An enzyme secreted from the host cells may conveniently be recovered from the culture medium by well-known procedures, including separating the cells from the medium by centrifugation or filtration, and precipitating proteinaceous components of the medium by means of a salt such as ammonium sulfate, followed by the use of chromatographic procedures such as ion exchange chromatography, affinity chromatography, or the like.

Fermentation, separation, and concentration techniques are well known in the art and conventional methods can be used in order to prepare a concentrated xylanase polypeptide-containing solution. After fermentation, a fermentation broth is obtained, the microbial cells and various suspended solids, including residual raw fermentation materials, are removed by conventional separation techniques in order to obtain a xylanase solution. Filtration, centrifugation, microfiltration, rotary vacuum drum filtration, ultrafiltration, centrifugation followed by ultra-filtration, extraction, or chromatography, or the like, are generally used.

It is desirable to concentrate a variant xylanase polypeptide-containing solution in order to optimize recovery. Use of unconcentrated solutions requires increased incubation time in order to collect the enriched or purified enzyme precipitate. The enzyme containing solution is concentrated using conventional concentration techniques until the desired enzyme level is obtained. Concentration of the enzyme containing solution may be achieved by any of the techniques discussed herein. Exemplary methods of enrichment and purification include but are not limited to rotary vacuum filtration and/or ultrafiltration.

In addition, concentration of desired protein product may be performed using, e.g., a precipitation agent, such as a metal halide precipitation agent. The metal halide precipitation agent, sodium chloride, can also be used as a preservative. The metal halide precipitation agent is used in an amount effective to precipitate the xylanase. The selection of at least an effective amount and an optimum amount of metal halide effective to cause precipitation of the enzyme, as well as the conditions of the precipitation for maximum recovery including incubation time, pH, temperature and concentration of enzyme, will be readily apparent to one of ordinary skill in the art, after routine testing. Generally, at least about 5% w/v (weight/volume) to about 25% w/v of metal halide is added to the concentrated enzyme solution, and usually at least 8% w/v.

Another alternative way to precipitate the enzyme is to use organic compounds. Exemplary organic compound precipitating agents include: 4-hydroxybenzoic acid, alkali metal salts of 4-hydroxybenzoic acid, alkyl esters of 4-hydroxybenzoic acid, and blends of two or more of these organic compounds. The addition of the organic compound precipitation agents can take place prior to, simultaneously with or subsequent to the addition of the metal halide precipitation agent, and the addition of both precipitation agents, organic compound and metal halide, may be carried out sequentially or simultaneously. Generally, the organic precipitation agents are selected from the group consisting of alkali metal salts of 4-hydroxybenzoic acid, such as sodium or potassium salts, and linear or branched alkyl esters of 4-hydroxybenzoic acid, wherein the alkyl group contains from 1 to 12 carbon atoms, and blends of two or more of these organic compounds. Additional organic compounds also include but are not limited to 4-hydroxybenzoic acid methyl ester (named methyl PARABEN), 4-hydroxybenzoic acid propyl ester (named propyl PARABEN). For further descriptions, see, e.g., U.S. Pat. No. 5,281,526. Addition of the organic compound precipitation agent provides the advantage of high flexibility of the precipitation conditions with respect to pH, temperature, variant xylanase concentration, precipitation agent concentration, and time of incubation. Generally, at least about 0.01% w/v and no more than about 0.3% w/v of organic compound precipitation agent is added to the concentrated enzyme solution.

After the incubation period, the enriched or purified enzyme is then separated from the dissociated pigment and other impurities and collected by conventional separation techniques, such as filtration, centrifugation, microfiltration, rotary vacuum filtration, ultrafiltration, press filtration, cross membrane microfiltration, cross flow membrane microfiltration, or the like. Further enrichment or purification of the enzyme precipitate can be obtained by washing the precipitate with water. For example, the enriched or purified enzyme precipitate is washed with water containing the metal halide precipitation agent, or with water containing the metal halide and the organic compound precipitation agents.

Also described herein is a recombinant microbial production host for expressing at least one polypeptide described herein, said recombinant microbial production host comprising a recombinant construct described herein. In another embodiment, this recombinant microbial production host is selected from the group consisting of bacteria, fungi and algae.

Expression will be understood to include any step involved in producing at least one polypeptide described herein including, but not limited to, transcription, post-transcriptional modification, translation, post-translation modification and secretion.

Techniques for modifying nucleic acid sequences utilizing cloning methods are well known in the art.

A polynucleotide encoding a xylanase can be manipulated in a variety of ways to provide for expression of the polynucleotide in a heterologous microbial host cell such as Bacillus or Trichoderma. Manipulation of the polynucleotide sequence prior to its insertion into a nucleic acid construct or vector may be desirable or necessary depending on the nucleic acid construct or vector or the heterologous microbial host cell. The techniques for modifying nucleotide sequences utilizing cloning methods are well known in the art.

Regulatory sequences are defined above. They include all components, which are necessary or advantageous for the expression of a xylanase. Each control sequence may be native or foreign to the nucleotide sequence encoding the xylanase. Such regulatory sequences include, but are not limited to, a leader, a polyadenylation sequence, a propeptide sequence, a promoter, a signal sequence and a transcription terminator. Regulatory sequences may be provided with linkers for the purpose of introducing specific restriction sites facilitating ligation or the regulatory sequences with the coding region of the nucleotide sequence encoding a xylanase.

A nucleic acid construct comprising a polynucleotide encoding a xylanase may be operably linked to one or more control sequences capable of directing the expression of the coding sequence in a heterologous microbial such as Bacillus host cell under conditions compatible with the control sequences.

Each control sequence may be native or foreign to the polynucleotide encoding a xylanase. Such control sequences include, but are not limited to, a leader, a promoter, a signal sequence, and a transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a xylanase.

The control sequence may be an appropriate promoter region, a nucleotide sequence that is recognized by a heterologous microbial host cell for expression of the polynucleotide encoding a xylanase. The promoter region contains transcription control sequences that mediate the expression of a xylanase. The promoter region may be any nucleotide sequence that shows transcriptional activity in a Bacillus host cell of choice and may be obtained from genes directing synthesis of extracellular or intracellular polypeptides having biological activity either homologous or heterologous to the Bacillus host cell.

The promoter region may comprise a single promoter or a combination of promoters. Where the promoter region comprises a combination of promoters, the promoters are preferably in tandem. A promoter of the promoter region can be any promoter that can initiate transcription of a polynucleotide encoding a polypeptide having biological activity in a heterologous microbial host cell of interest. The promoter may be native, foreign, or a combination thereof, to the nucleotide sequence encoding a polypeptide having biological activity. Such a promoter can be obtained from genes directing synthesis of extracellular or intracellular polypeptides having biological activity either homologous or heterologous to the heterologous microbial host cell.

Thus, in certain embodiments, the promoter region comprises a promoter obtained from a bacterial source. In other embodiments, the promoter region comprises a promoter obtained from a Gram positive or Gram-negative bacterium. Gram positive bacteria include, but are not limited to, Bacillus, Streptococcus, Streptomyces, Staphylococcus, Enterococcus, Lactobacillus, Lactococcus, Clostridium, Geobacillus, and Oceanobacillus. Gram negative bacteria include, but are not limited to, E. coli, Pseudomonas, Salmonella, Campylobacter, Helicobacter, Flavobacterium, Fusobacterium, Ilyobacter, Neisseria, and Ureaplasma.

The promoter region may comprise a promoter obtained from a Bacillus strain (e.g., Bacillus agaradherens, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis); or from a Streptomyces strain (e.g., Streptomyces lividans or Streptomyces murinus).

The promoter region may comprise a promoter that is a “consensus” promoter having the sequence TTGACA for the “−35” region and TATAAT for the “−10” region. The consensus promoter may be obtained from any promoter that can function in a Bacillus host cell. The construction of a “consensus” promoter may be accomplished by site-directed mutagenesis using methods well known in the art to create a promoter that conforms more perfectly to the established consensus sequences for the “−10” and “−35” regions of the vegetative “sigma A-type” promoters for Bacillus subtilis (Voskuil et al., 1995, Molecular Microbiology 17: 271-279).

A control sequence may also be a suitable transcription terminator sequence, such as a sequence recognized by a Bacillus host cell to terminate transcription. The terminator sequence is operably linked to the 3′ terminus of the nucleotide sequence encoding a xylanase. Any terminator that is functional in the Bacillus host cell may be used.

The control sequence may also be a suitable leader sequence, a non-translated region of a mRNA that is important for translation by a Bacillus host cell. The leader sequence is operably linked to the 5′ terminus of the nucleotide sequence directing synthesis of the polypeptide having biological activity. Any leader sequence that is functional in a Bacillus host cell of choice may be used in the present invention.

The control sequence may also be a mRNA stabilizing sequence. The term “mRNA stabilizing sequence” is defined herein as a sequence located downstream of a promoter region and upstream of a coding sequence of a polynucleotide encoding a xylanase to which the promoter region is operably linked, such that all mRNAs synthesized from the promoter region may be processed to generate mRNA transcripts with a stabilizer sequence at the 5′ end of the transcripts. For example, the presence of such a stabilizer sequence at the 5′ end of the mRNA transcripts increases their half-life (Agaisse and Lereclus, 1994, supra, Hue et al., 1995, Journal of Bacteriology 177: 3465-3471). The mRNA processing/stabilizing sequence is complementary to the 3′ extremity of bacterial 16S ribosomal RNA. In certain embodiments, the mRNA processing/stabilizing sequence generates essentially single-size transcripts with a stabilizing sequence at the 5′ end of the transcripts. The mRNA processing/stabilizing sequence is preferably one, which is complementary to the 3′ extremity of a bacterial 16S ribosomal RNA. See, U.S. Pat. Nos. 6,255,076 and 5,955,310.

The nucleic acid construct can then be introduced into a Bacillus host cell using methods known in the art or those methods described herein for introducing and expressing a xylanase.

A nucleic acid construct comprising a DNA of interest encoding a protein of interest can also be constructed similarly as described above.

For obtaining secretion of the protein of interest of the introduced DNA, the control sequence may also comprise a signal peptide coding region, which codes for an amino acid sequence linked to the amino terminus of a polypeptide that can direct the expressed polypeptide into the cell's secretory pathway. The signal peptide coding region may be native to the polypeptide or may be obtained from foreign sources. The 5′ end of the coding sequence of the nucleotide sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to that portion of the coding sequence that encodes the secreted polypeptide. The foreign signal peptide coding region may be required where the coding sequence does not normally contain a signal peptide coding region. Alternatively, the foreign signal peptide coding region may simply replace the natural signal peptide coding region in order to obtain enhanced secretion of the polypeptide relative to the natural signal peptide coding region normally associated with the coding sequence. The signal peptide coding region may be obtained from an amylase or a xylanase gene from a Bacillus species. However, any signal peptide coding region capable of directing the expressed polypeptide into the secretory pathway of a Bacillus host cell of choice may be used in the present invention.

An effective signal peptide coding region for a Bacillus host cell, is the signal peptide coding region obtained from the maltogenic amylase gene from Bacillus NCIB 11837, the Bacillus stearothermophilus alpha-amylase gene, the Bacillus licheniformis subtilisin gene, the Bacillus licheniformis beta-lactamase gene, the Bacillus stearothermophilus neutral protease genes (nprT, nprS, nprM), and the Bacillus subtilis prsA gene.

Thus, a polynucleotide construct comprising a nucleic acid encoding a xylanase construct comprising a nucleic acid encoding a polypeptide of interest (POI) can be constructed such that it is expressed by a host cell. Because of the known degeneracies in the genetic code, different polynucleotides encoding an identical amino acid sequence can be designed and made with routine skills in the art. For example, codon optimizations can be applied to optimize production in a particular host cell.

Nucleic acids encoding proteins of interest can be incorporated into a vector, wherein the vector can be transferred into a host cell using well-known transformation techniques, such as those disclosed herein.

The vector may be any vector that can be transformed into and replicated within a host cell. For example, a vector comprising a nucleic acid encoding a POI can be transformed and replicated in a bacterial host cell as a means of propagating and amplifying the vector. The vector also may be transformed into a Bacillus expression host of the disclosure, so that the protein encoding nucleic acid (e.g., an ORF) can be expressed as a functional protein.

A representative vector which can be modified with routine skill to comprise and express a nucleic acid encoding a POI is vector p2JM103BBI.

A polynucleotide encoding a xylanase or a POI can be operably linked to a suitable promoter, which allows transcription in the host cell. The promoter may be any nucleic acid sequence that shows transcriptional activity in the host cell of choice and may be derived from genes encoding proteins either homologous or heterologous to the host cell. Means of assessing promoter activity/strength are routine for the skilled artisan.

Examples of suitable promoters for directing the transcription of a polynucleotide sequence encoding comS1 polypeptide or a POI of the disclosure, especially in a bacterial host, include the promoter of the lac operon of E. coli, the Streptomyces coelicolor agarase gene dagA or celA promoters, the promoters of the Bacillus licheniformis alpha-amylase gene (amyL), the promoters of the Bacillus stearothermophilus maltogenic amylase gene (amyM), the promoters of the Bacillus amyloliquefaciens alpha-amylase (amyQ), the promoters of the Bacillus subtilis xylA and xylB genes, and the like.

A promoter for directing the transcription of a polynucleotide sequence encoding a POI can be a wild-type aprE promoter, a mutant aprE promoter or a consensus aprE promoter set forth in PCT International Publication No. WO2001/51643. In certain other embodiments, a promoter for directing the transcription of a polynucleotide sequence encoding a POI is a wild-type spoVG promoter, a mutant spoVG promoter, or a consensus spoVG promoter (Frisby and Zuber, 1991).

A promoter for directing the transcription of the polynucleotide sequence encoding a xylanase or a POI is a ribosomal promoter such as a ribosomal RNA promoter or a ribosomal protein promoter. The ribosomal RNA promoter can be a rrn promoter derived from B. subtilis, more particularly, the rrn promoter can be a rrnB, rrnI or rrnE ribosomal promoter from B. subtilis. In certain embodiments, the ribosomal RNA promoter is a P2 rrnI promoter from B. subtilis set forth in PCT International Publication No. WO2013/086219.

A suitable vector may further comprise a nucleic acid sequence enabling the vector to replicate in the host cell. Examples of such enabling sequences include the origins of replication of plasmids pUC19, pACYC177, pUB110, pE194, pAMB1, pIJ702, and the like.

A suitable vector may also comprise a selectable marker, e.g., a gene the product of which complements a defect in the isolated host cell, such as the dal genes from B. subtilis or B. licheniformis; or a gene that confers antibiotic resistance such as, e.g., ampicillin resistance, kanamycin resistance, chloramphenicol resistance, tetracycline resistance and the like.

A suitable expression vector typically includes components of a cloning vector, such as, for example, an element that permits autonomous replication of the vector in the selected host organism and one or more phenotypically detectable markers for selection purposes. Expression vectors typically also comprise control nucleotide sequences such as, for example, promoter, operator, ribosome binding site, translation initiation signal and optionally, a repressor gene, one or more activator genes sequences, or the like.

Additionally, a suitable expression vector may further comprise a sequence coding for an amino acid sequence capable of targeting the protein of interest to a host cell organelle such as a peroxisome, or to a particular host cell compartment. Such a targeting sequence may be, for example, the amino acid sequence “SKL”. For expression under the direction of control sequences, the nucleic acid sequence of the protein of interest can be operably linked to the control sequences in a suitable manner such that the expression takes place.

Protocols, such as described herein, used to ligate the DNA construct encoding a protein of interest, promoters, terminators and/or other elements, and to insert them into suitable vectors containing the information necessary for replication, are well known to persons skilled in the art.

An isolated cell, either comprising a polynucleotide construct or an expression vector, is advantageously used as a host cell in the recombinant production of a POI. The cell may be transformed with the DNA construct encoding the POI, conveniently by integrating the construct (in one or more copies) into the host chromosome. Integration is generally deemed an advantage, as the DNA sequence thus introduced is more likely to be stably maintained in the cell. Integration of the DNA constructs into the host chromosome may be performed applying conventional methods, for example, by homologous or heterologous recombination. For example, PCT International Publication No. WO2002/14490 describes methods of Bacillus transformation, transformants thereof and libraries thereof. Alternatively, the cell may be transformed with an expression vector as described above in connection with the different types of host cells.

Sometimes it is advantageous to delete genes from expression hosts, where the gene deficiency can be cured by an expression vector. Known methods may be used to obtain a bacterial host cell having one or more inactivated genes. Gene inactivation may be accomplished by complete or partial deletion, by insertional inactivation or by any other means that renders a gene nonfunctional for its intended purpose, such that the gene is prevented from expression of a functional protein.

Techniques for transformation of bacteria and culturing the bacteria are standard and well known in the art. They can be used to transform the improved hosts of the present invention for the production of recombinant proteins of interest. Introduction of a DNA construct or vector into a host cell includes techniques such as transformation, electroporation, nuclear microinjection, transduction, transfection (e.g., lipofection mediated and DEAE-Dextrin mediated transfection), incubation with calcium phosphate DNA precipitate, high velocity bombardment with DNA-coated microprojectiles, gene gun or biolistic transformation and protoplast fusion, and the like. Transformation and expression methods for bacteria are also disclosed in Brigidi et al. (1990).

Methods for transforming nucleic acids into filamentous fungi such as Aspergillus spp., e.g., A. oryzae or A. niger, H. grisea, H. insolens, and T. reesei. are well known in the art. A suitable procedure for transformation of Aspergillus host cells is described, for example, in EP238023. A suitable procedure for transformation of Trichoderma host cells is described, for example, in Steiger et al 2011, Appl. Environ. Microbiol. 77:114-121.

The choice of a production host can be any suitable microorganism such as bacteria, fungi and algae.

Typically, the choice will depend upon the gene encoding the xylanase and its source.

Introduction of a DNA construct or vector into a host cell includes techniques such as transformation; electroporation; nuclear microinjection; transduction; transfection, (e.g., lipofection mediated and DEAE-Dextrin mediated transfection); incubation with calcium phosphate DNA precipitate; high velocity bombardment with DNA-coated microprojectiles; and protoplast fusion. Basic texts disclosing the general methods that can be used include Sambrook et al., Molecular Cloning, A Laboratory Manual (2nd ed. 1989); Kriegler, Gene Transfer and Expression: A Laboratory Manual (1990); and Ausubel et al., eds., Current Protocols in Molecular Biology (1994)). The methods of transformation of the present invention may result in the stable integration of all or part of the transformation vector into the genome of a host cell, such as a filamentous fungal host cell. However, transformation resulting in the maintenance of a self-replicating extra-chromosomal transformation vector is also contemplated.

Many standard transfection methods can be used to produce bacterial and filamentous fungal (e.g. Aspergillus or Trichoderma) cell lines that express large quantities of the xylanase. Some of the published methods for the introduction of DNA constructs into cellulase-producing strains of Trichoderma include Lorito, Hayes, DiPietro and Harman, (1993) Curr. Genet. 24: 349-356; Goldman, VanMontagu and Herrera-Estrella, (1990) Curr. Genet. 17:169-174; and Penttila, Nevalainen, Ratto, Salminen and Knowles, (1987) Gene 6: 155-164, also see U.S. Pat. Nos. 6,022,725; 6,268,328 and Nevalainen et al., “The Molecular Biology of Trichoderma and its Application to the Expression of Both Homologous and Heterologous Genes” in Molecular Industrial Mycology, Eds, Leong and Berka, Marcel Dekker Inc., NY (1992) pp 129-148; for Aspergillus include Yelton, Hamer and Timberlake, (1984) Proc. Natl. Acad. Sci. USA 81: 1470-1474, for Fusarium include Bajar, Podila and Kolattukudy, (1991) Proc. Natl. Acad. Sci. USA 88: 8202-8212, for Streptomyces include Hopwood et al., 1985, Genetic Manipulation of Streptomyces: Laboratory Manual, The John Innes Foundation, Norwich, UK and Fernandez-Abalos et al., Microbiol 149:1623-1632 (2003) and for Bacillus include Brigidi, DeRossi, Bertarini, Riccardi and Matteuzzi, (1990) FEMS Microbiol. Lett. 55: 135-138).

However, any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well-known methods for introducing cloned genomic DNA, cDNA, synthetic DNA or other foreign genetic material into a host cell (see, e.g., Sambrook et al., supra). Also of use is the Agrobacterium-mediated transfection method described in U.S. Pat. No. 6,255,115. It is only necessary that the particular genetic engineering procedure used be capable of successfully introducing at least one gene into the host cell capable of expressing the gene.

After the expression vector is introduced into the cells, the transfected or transformed cells are cultured under conditions favoring expression of genes under control of the promoter sequences.

The medium used to cultivate the cells may be any conventional medium suitable for growing the host cell and obtaining expression of a polypeptide having xylanase activity. Suitable media and media components are available from commercial suppliers or may be prepared according to published recipes (e.g., as described in catalogues of the American Type Culture Collection).

A polypeptide having xylanase activity secreted from the host cells can be used, with minimal post-production processing, as a whole broth preparation.

Depending upon the host cell used post-transcriptional and/or post-translational modifications may be made. One non-limiting example of a post-transcriptional and/or post-translational modification is “clipping” or “truncation” of a polypeptide. For example, this may result in taking a xylanase from an inactive or substantially inactive state to an active state as in the case of a pro-peptide undergoing further post-translational processing to a mature peptide having the enzymatic activity. In another instance, this clipping may result in taking a mature xylanase polypeptide and further removing N or C-terminal amino acids to generate truncated forms of the xylanase that retain enzymatic activity.

Other examples of post-transcriptional or post-translational modifications include, but are not limited to, myristoylation, glycosylation, truncation, lipidation and tyrosine, serine or threonine phosphorylation. The skilled person will appreciate that the type of post-transcriptional or post-translational modifications that a protein may undergo may depend on the host organism in which the protein is expressed.

In some embodiments, the preparation of a spent whole fermentation broth of a recombinant microorganism can be achieved using any cultivation method known in the art resulting in the expression of a xylanase, i.e, a polypeptide having xylanase activity.

Fermentation may, therefore, be understood as comprising shake flask cultivation, small- or large-scale fermentation (including continuous, batch, fed-batch, or solid-state fermentations) in laboratory or industrial fermenters performed in a suitable medium and under conditions allowing the xylanase to be expressed or isolated. The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

Host cells may be cultured under suitable conditions that allow expression of a xylanase. Expression of the enzymes may be constitutive such that they are continually produced, or inducible, requiring a stimulus to initiate expression. In the case of inducible expression, protein production can be initiated when required by, for example, addition of an inducer substance to the culture medium, for example dexamethasone or IPTG or sophorose.

Any of the fermentation methods well known in the art can suitably be used to ferment the transformed or the derivative fungal strain as described above. In some embodiments, fungal cells are grown under batch or continuous fermentation conditions.

A classical batch fermentation is a closed system, where the composition of the medium is set at the beginning of the fermentation, and the composition is not altered during the fermentation. At the beginning of the fermentation, the medium is inoculated with the desired organism(s). In other words, the entire fermentation process takes place without addition of any components to the fermentation system throughout.

Alternatively, a batch fermentation qualifies as a “batch” with respect to the addition of the carbon source. Moreover, attempts are often made to control factors such as pH and oxygen concentration throughout the fermentation process. Typically, the metabolite and biomass compositions of the batch system change constantly up to the time the fermentation is stopped. Within batch cultures, cells progress through a static lag phase to a high growth log phase and finally to a stationary phase, where growth rate is diminished or halted. Left untreated, cells in the stationary phase would eventually die. In general, cells in log phase are responsible for the bulk of production of product. A suitable variation on the standard batch system is the “fed-batch fermentation” system. In this variation of a typical batch system, the substrate is added in increments as the fermentation progresses. Fed-batch systems are useful when it is known that catabolite repression would inhibit the metabolism of the cells, and/or where it is desirable to have limited amounts of substrates in the fermentation medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are well known in the art.

Continuous fermentation is another known method of fermentation. It is an open system where a defined fermentation medium is added continuously to a bioreactor, and an equal amount of conditioned medium is removed simultaneously for processing. Continuous fermentation generally maintains the cultures at a constant density, where cells are maintained primarily in log phase growth. Continuous fermentation allows for the modulation of one or more factors that affect cell growth and/or product concentration. For example, a limiting nutrient, such as the carbon source or nitrogen source, can be maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

Separation and concentration techniques are known in the art and conventional methods can be used to prepare a concentrated solution or broth comprising a xylanase polypeptide of the invention.

After fermentation, a fermentation broth is obtained, the microbial cells and various suspended solids, including residual raw fermentation materials, are removed by conventional separation techniques in order to obtain a xylanase solution. Filtration, centrifugation, microfiltration, rotary vacuum drum filtration, ultrafiltration, centrifugation followed by ultra-filtration, extraction, or chromatography, or the like, are generally used.

It may at times be desirable to concentrate a solution or broth comprising an xylanase polypeptide to optimize recovery. Use of un-concentrated solutions or broth would typically increase incubation time in order to collect the enriched or purified enzyme precipitate.

The enzyme-containing solution can be concentrated using conventional concentration techniques until the desired enzyme level is obtained. Concentration of the enzyme containing solution may be achieved by any of the techniques discussed herein. Examples of methods of enrichment and purification include but are not limited to rotary vacuum filtration and/or ultrafiltration.

The xylanase-containing solution or broth may be concentrated until such time the enzyme activity of the concentrated a xylanase polypeptide-containing solution or broth is at a desired level.

Concentration may be performed using, e.g., a precipitation agent, such as a metal halide precipitation agent. Metal halide precipitation agents include but are not limited to alkali metal chlorides, alkali metal bromides and blends of two or more of these metal halides.

Exemplary metal halides include sodium chloride, potassium chloride, sodium bromide, potassium bromide and blends of two or more of these metal halides. The metal halide precipitation agent, sodium chloride, can also be used as a preservative. For production scale recovery, xylanase polypeptides can be enriched or partially purified as generally described above by removing cells via flocculation with polymers. Alternatively, the enzyme can be enriched or purified by microfiltration followed by concentration by ultrafiltration using available membranes and equipment. However, for some applications, the enzyme does not need to be enriched or purified, and whole broth culture can be lysed and used without further treatment. The enzyme can then be processed, for example, into granules.

Xylanases may be isolated or purified in a variety of ways known to those skilled in the art depending on what other components are present in the sample. Standard purification methods include, but are not limited to, chromatography (e.g., ion exchange, affinity, hydrophobic, chromatofocusing, immunological and size exclusion), electrophoretic procedures (e.g., preparative isoelectric focusing), differential solubility (e.g., ammonium sulfate precipitation), extraction microfiltration, two phase separation. For example, the protein of interest may be purified using a standard anti-protein of interest antibody column. Ultrafiltration and diafiltration techniques, in conjunction with protein concentration, are also useful. For general guidance in suitable purification techniques, see Scopes, Protein purification (1982). The degree of purification necessary will vary depending on the use of the protein of interest. In some instances, no purification will be necessary.

Assays for detecting and measuring the enzymatic activity of an enzyme, such as a xylanase polypeptide, are well known. Various assays for detecting and measuring activity of xylanases, are also known to those of ordinary skill in the art.

Xylanase activity may be determined using soluble 4-O-Methyl-D-glucurono-D-xylan dyed with Remazol brilliant blue R (RBB-Xylan) as substrate. After precipitation of undegraded high molecular weight RBB-Xylan, the absorbance of the supernatant is proportional to the production of low molecular weight fragments by enzyme treatment. Another method to measure xylanase activity is to measure their ability to degrade the water unextractable arabinoxylans (WU-AX) in corn DDGS or rice bran. For example, a 5% or 10% substrate solution of corn DDGS or rice bran, ground to a particle size <212 μm and hydrated in buffer to the desired pH, such as pH 6, can be used. Following incubation with the xylanase enzyme, the total amount of C5 sugar units in solution can be measured as xylose equivalents by the Douglas method using a continuous flow injection apparatus such as one from SKALAR Analytical, as described by Rouau X & Surget A (1994). The combination of heat and low pH will lead to a decomposition of arabinoxylan into the pentose mono-sugars, arabinose and xylose, which will further dehydrate into furfural. By reaction with phloroglucinol a colored complex is formed. By measuring the absorbance at 550 nm with 510 nm as reference wavelength, the concentration of pentose mono-sugars in solution can be measured as xylose equivalents using a xylose standard curve. The extracted arabinoxylan can be determined as the mass of the hydrated xylose equivalents per substrate mass. The results are reported as the increase in extractable arabinoxylan calculated as the difference between extracted arabinoxylan for the xylanase enzyme treated sample and for the blank sample.

In one embodiment, there is disclosed an additive for animal feed comprising corn or rice, the feed additive comprising at least one enzyme with glucuronoxylanase activity and at least one enzyme having endo-beta-1,4-xylanase activity wherein degradation of insoluble glucuronoxylan is greater than if either enzyme was used alone.

The xylanase with glucuronoxylanase activity is derived from Bacillus or Paenibacillus sp. This xylanase is currently identified as a member of the GH30 family.

The xylanase having endo-beta-1,4-xylanase activity is derived from Fusarium sp. This xylanase is currently identified as a member of the GH10 family.

In another embodiment, at least one of the xylanases disclosed herein can be recombinantly produced as discussed above.

In still another embodiment, there is disclosed a feed additive comprising at least one enzyme with glucuronoxylanase activity and at least one enzyme having endo-beta-1,4-xylanase activity wherein said combination is better in stimulating growth of beneficial bacteria in a digestive tract of a monogastric animal fed a corn based diet when compared to the use of the xylanase having endo-beta-1,4-xylanase activity alone.

Gut flora, gut microbiota or gastrointestinal microbiota is the complex community of microorganisms that live in the digestive tracts of humans and other animals. The relationship between some gut flora and animals is not merely commensal (i.e., a non-harmful coexistence), but rather a mutualistic relationship. Some animal gut microorganisms benefit the animal by fermenting dietary fiber into short chain fatty acids such as acetic acid, propionic acid and/or butyric acid which are then absorbed by the animal.

In another aspect, there is disclosed a feed additive comprising at least one enzyme with glucuronoxylanase activity and at least one enzyme having endo-beta-1,4-xylanase activity wherein the combination is capable of increasing production of at least one short chain fatty acid in a monogastric animal fed a corn-based diet when compared to the use of the xylanase having endo-beta-1,4-xylanase activity alone.

The short chain fatty acid is selected from the group consisting of acetic acid, propionic acid or butyric acid.

In still another aspect, any of the feed additives described herein may further comprise one or more enzymes selected from, but not limited to, enzymes such as amylase, protease, endo-glucanase, cellulase, phytase, etc.

Any of these enzymes can be used in an amount ranging from 0.1 to 500 micrograms/g feed or feedstock.

Amylases such as alpha-amylases (alpha-1,4-glucan-4-glucanohydrolase, EC 3.2.1.1.) hydrolyze internal alpha-1,4-glucosidic linkages in starch, largely at random to produce smaller molecular weight dextrans. These polypeptides are used, inter alia, in starch processing and in alcohol production. Any alpha-amylases can be used, e.g., those described in U.S. Pat. Nos. 8,927,250 and 7,354,752.

Phytase refers to a protein or polypeptide which is capable of catalyzing the hydrolysis of phytate to (1) myo-inositol and/or (2) mono-, di-, tri-, tetra-, and/or penta-phosphates thereof and (3) inorganic phosphate. For example, enzymes having catalytic activity as defined in Enzyme Commission EC number 3.1.3.8 or EC number 3.1.3.26. Any phytase can be used such as described in U.S. Pat. Nos. 8,144,046, 8,673,609, and 8,053,221.

Glucanases are enzymes that break down glucan, a polysaccharide made several glucose sub-units. As they perform hydrolysis of the glucosidic bond, they are hydrolases. Beta-glucanase enzymes (EC 3.2.1.4) digests fiber. It helps in the breakdown of plant walls (cellulose).

Cellulases are any of several enzymes produced by fungi, bacteria and protozoans that catalyze cellulolysis, the decomposition of cellulose and of some related polysaccharides. The name is also used for any naturally-occurring mixture or complex of various such enzymes, that act serially or synergistically to decompose cellulosic material. Any cellulases can be used that are suitable for animal feed.

A “protease” is any protein or polypeptide domain of derived from a microorganism, e.g., a fungus, bacterium, or from a plant or animal, and that has the ability to catalyze cleavage of peptide bonds at one or more of various positions of a protein backbone (e.g., E.C. 3.4). The terms “protease”, “peptidase” and “proteinase” can be used interchangeably. Proteases can be found in animals, plants, fungi, bacteria, archaea and viruses. Proteolysis can be achieved by enzymes currently classified into six broad groups: aspartyl proteases, cysteine proteases, serine proteases, threonine proteases, glutamic proteases, and metalloproteases. Any protease can be used that is suitable for animal feed.

In still another aspect the feed additive may also comprise at least one DFM either alone or in combination with at least one other enzyme as described above.

At least one DFM may comprise at least one viable microorganism such as a viable bacterial strain or a viable yeast or a viable fungi. Preferably, the DFM comprises at least one viable bacteria.

It is possible that the DFM may be a spore forming bacterial strain and hence the term DFM may be comprised of or contain spores, e.g. bacterial spores. Thus, the term “viable microorganism” as used herein may include microbial spores, such as endospores or conidia. Alternatively, the DFM in the feed additive composition described herein may not comprise of or may not contain microbial spores, e.g. endospores or conidia.

The microorganism may be a naturally-occurring microorganism or it may be a transformed microorganism.

A DFM as described herein may comprise microorganisms from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera and combinations thereof.

Preferably, the DFM comprises one or more bacterial strains selected from the following Bacillus spp: Bacillus subtilis, Bacillus cereus, Bacillus licheniformis, Bacillus pumilis and Bacillus amyloliquefaciens.

The genus “Bacillus”, as used herein, includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. gibsonii, B. pumilis and B. thuringiensis. It is recognized that the genus Bacillus continues to undergo taxonomical reorganization. Thus, it is intended that the genus include species that have been reclassified, including but not limited to such organisms as Bacillus stearothermophilus, which is now named “Geobacillus stearothermophilus”, or Bacillus polymyxa, which is now “Paenibacillus polymyxa” The production of resistant endospores under stressful environmental conditions is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

In another aspect, the DFM may be further combined with the following Lactococcus spp: Lactococcus cremoris and Lactococcus lactis and combinations thereof.

The DFM may be further combined with the following Lactobacillus spp: Lactobacillus buchneri, Lactobacillus acidophilus, Lactobacillus casei, Lactobacillus kefiri, Lactobacillus bifidus, Lactobacillus brevis, Lactobacillus helveticus, Lactobacillus paracasei, Lactobacillus rhamnosus, Lactobacillus salivarius, Lactobacillus curvatus, Lactobacillus bulgaricus, Lactobacillus sakei, Lactobacillus reuteri, Lactobacillus fermentum, Lactobacillus farciminis, Lactobacillus lactis, Lactobacillus delbreuckii, Lactobacillus plantarum, Lactobacillus paraplantarum, Lactobacillus farciminis, Lactobacillus rhamnosus, Lactobacillus crispatus, Lactobacillus gasseri, Lactobacillus johnsonii and Lactobacillus jensenii, and combinations of any thereof.

In still another aspect, the DFM may be further combined with the following Bifidobacteria spp: Bifidobacterium lactis, Bifidobacterium bifidium, Bifidobacterium longum, Bifidobacterium animalis, Bifidobacterium breve, Bifidobacterium infantis, Bifidobacterium catenulatum, Bifidobacterium pseudocatenulatum, Bifidobacterium adolescentis, and Bifidobacterium angulatum, and combinations of any thereof.

There can be mentioned bacteria of the following species: Bacillus subtilis, Bacillus licheniformis, Bacillus amyloliquefaciens, Bacillus pumilis, Enterococcus, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Bacillus subtilis, Propionibacterium thoenii, Lactobacillus farciminis, Lactobacillus rhamnosus, Megasphaera elsdenii, Clostridium butyricum, Bifidobacterium animalis ssp. animalis, Lactobacillus reuteri, Bacillus cereus, Lactobacillus salivarius ssp. Salivarius, Propionibacteria sp and combinations thereof.

A direct-fed microbial described herein comprising one or more bacterial strains may be of the same type (genus, species and strain) or may comprise a mixture of genera, species and/or strains.

Alternatively, a DFM may be combined with one or more of the products or the microorganisms contained in those products disclosed in WO2012110778, and summarized as follows: Bacillus subtilis strain 2084 Accession No. NRRI B-50013, Bacillus subtilis strain LSSAO1 Accession No. NRRL B-50104, and Bacillus subtilis strain 15A-P4 ATCC Accession No. PTA-6507 (from Enviva Pro®. (formerly known as Avicorr®); Bacillus subtilis Strain C3102 (from Calsporin®); Bacillus subtilis Strain PB6 (from Clostat®); Bacillus pumilis (8G-134); Enterococcus NCIMB 10415 (SF68) (from Cylactin®); Bacillus subtilis Strain C3102 (from Gallipro® & GalliproMax®); Bacillus licheniformis (from Gallipro®Tect®); Enterococcus and Pediococcus (from Poultry Star®); Lactobacillus, Bifidobacterium and/or Enterococcus from Protexin®); Bacillus subtilis strain QST 713 (from Proflora®); Bacillus amyloliquefaciens CECT-5940 (from Ecobiol® & Ecobiol® Plus); Enterococcus faecium SF68 (from Fortiflora®); Bacillus subtilis and Bacillus licheniformis (from BioPlus2B®); Lactic acid bacteria 7 Enterococcus faecium (from Lactiferm®); Bacillus strain (from CSI®); Saccharomyces cerevisiae (from Yea-Sacc®); Enterococcus (from Biomin IMB52®); Pediococcus acidilactici, Enterococcus, Bifidobacterium animalis ssp. animalis, Lactobacillus reuteri, Lactobacillus salivarius ssp. salivarius (from Biomin C5®); Lactobacillus farciminis (from Biacton®); Enterococcus (from Oralin E1707®); Enterococcus (2 strains), Lactococcus lactis DSM 1103 (from Probios-pioneer PDFM®); Lactobacillus rhamnosus and Lactobacillus farciminis (from Sorbiflore®); Bacillus subtilis (from Animavit®); Enterococcus (from Bonvital®); Saccharomyces cerevisiae (from Levucell SB 20®); Saccharomyces cerevisiae (from Levucell SC 0 & SC10® ME); Pediococcus acidilacti (from Bactocell); Saccharomyces cerevisiae (from ActiSaf® (formerly BioSaf®)); Saccharomyces cerevisiae NCYC Sc47 (from Actisaf® SC47); Clostridium butyricum (from Miya-Gold®); Enterococcus (from Fecinor and Fecinor Plus®); Saccharomyces cerevisiae NCYC R-625 (from InteSwine®); Saccharomyces cerevisia (from BioSprint®); Enterococcus and Lactobacillus rhamnosus (from Provita®); Bacillus subtilis and Aspergillus oryzae (from PepSoyGen-C®); Bacillus cereus (from Toyocerin®); Bacillus cereus var. toyoi NCIMB 40112/CNCM I-1012 (from TOYOCERIN®), or other DFMs such as Bacillus licheniformis and Bacillus subtilis (from BioPlus® YC) and Bacillus subtilis (from GalliPro®).

The DFM may be combined with Enviva® PRO which is commercially available from Danisco A/S. Enviva Pro® is a combination of Bacillus strain 2084 Accession No. NRRI B-50013, Bacillus strain LSSAO1 Accession No. NRRL B-50104 and Bacillus strain 15A-P4 ATCC Accession No. PTA-6507 (as taught in U.S. Pat. No. 7,754,469 B—incorporated herein by reference).

It is also possible to combine the DFM described herein with a yeast from the genera: Saccharomyces spp.

Preferably, the DFM described herein comprises microorganisms which are generally recognized as safe (GRAS) and, preferably are GRAS-approved.

A person of ordinary skill in the art will readily be aware of specific species and/or strains of microorganisms from within the genera described herein which are used in the food and/or agricultural industries and which are generally considered suitable for animal consumption.

In some embodiments, it is important that the DFM be heat tolerant, i.e. is thermotolerant. This is particularly the case when the feed is pelleted. Therefore, in another embodiment, the DFM may be a thermotolerant microorganism, such as a thermotolerant bacteria,_including for example Bacillus spp.

In other aspects, it may be desirable that the DFM comprises a spore producing bacteria, such as Bacilli, e.g. Bacillus spp. Bacilli are able to form stable endospores when conditions for growth are unfavorable and are very resistant to heat, pH, moisture and disinfectants.

The DFM described herein may decrease or prevent intestinal establishment of pathogenic microorganism (such as Clostridium perfringens and/or E. coli and/or Salmonella spp and/or Campylobacter spp.). In other words, the DFM may be antipathogenic. The term “antipathogenic” as used herein means the DFM counters an effect (negative effect) of a pathogen.

As described above, the DFM may be any suitable DFM. For example, the following assay “DFM ASSAY” may be used to determine the suitability of a microorganism to be a DFM. The DFM assay as used herein is explained in more detail in US2009/0280090. For avoidance of doubt, the DFM selected as an inhibitory strain (or an antipathogenic DFM) in accordance with the “DFM ASSAY” taught herein is a suitable DFM for use in accordance with the present disclosure, i.e. in the feed additive composition according to the present disclosure.

Tubes were seeded each with a representative pathogen (e.g., bacteria) from a representative cluster.

Supernatant from a potential DFM, grown aerobically or anaerobically, is added to the seeded tubes (except for the control to which no supernatant is added) and incubated. After incubation, the optical density (OD) of the control and supernatant treated tubes was measured for each pathogen.

Colonies of (potential DFM) strains that produced a lowered OD compared with the control (which did not contain any supernatant) can then be classified as an inhibitory strain (or an antipathogenic DFM). Thus, The DFM assay as used herein is explained in more detail in US2009/0280090.

Preferably, a representative pathogen used in this DFM assay can be one (or more) of the following: Clostridium, such as Clostridium perfringens and/or Clostridium difficile, and/or E. coli and/or Salmonella spp and/or Campylobacter spp. In one preferred embodiment the assay is conducted with one or more of Clostridium perfringens and/or Clostridium difficile and/or E. coli, preferably Clostridium perfringens and/or Clostridium difficile, more preferably Clostridium perfringens.

Antipathogenic DFMs include one or more of the following bacteria and are described in WO2013029013:

Bacillus subtilis strain 3BP5 Accession No. NRRL B-50510,
Bacillus amyloliquefaciens strain 918 ATCC Accession No. NRRL B-50508, and
Bacillus amyloliquefaciens strain 1013 ATCC Accession No. NRRL B-50509.

DFMs may be prepared as culture(s) and carrier(s) (where used) and can be added to a ribbon or paddle mixer and mixed for about 15 minutes, although the timing can be increased or decreased. The components are blended such that a uniform mixture of the cultures and carriers result. The final product is preferably a dry, flowable powder. The DFM(s) comprising one or more bacterial strains can then be added to animal feed or a feed premix, added to an animal's water, or administered in other ways known in the art (preferably simultaneously with the enzymes described herein.

Inclusion of the individual strains in the DFM mixture can be in proportions varying from 1% to 99% and, preferably, from 25% to 75%

Suitable dosages of the DFM in animal feed may range from about 1×10³ CFU/g feed to about 1×10¹⁰ CFU/g feed, suitably between about 1×10⁴ CFU/g feed to about 1×10⁸ CFU/g feed, suitably between about 7.5×10⁴ CFU/g feed to about 1×10⁷ CFU/g feed.

In another aspect, the DFM may be dosed in feedstuff at more than about 1×10³ CFU/g feed, suitably more than about 1×10⁴ CFU/g feed, suitably more than about 5×10⁴ CFU/g feed, or suitably more than about 1×10⁵ CFU/g feed.

The DFM may be dosed in a feed additive composition from about 1×10³ CFU/g composition to about 1×10¹³ CFU/g composition, preferably 1×10⁵ CFU/g composition to about 1×10¹³ CFU/g composition, more preferably between about 1×10⁶ CFU/g composition to about 1×10¹² CFU/g composition, and most preferably between about 3.75×10⁷ CFU/g composition to about 1×10¹¹ CFU/g composition. In another aspect, the DFM may be dosed in a feed additive composition at more than about 1×10⁵ CFU/g composition, preferably more than about 1×10⁶ CFU/g composition, and most preferably more than about 3.75×10⁷ CFU/g composition. In one embodiment, the DFM is dosed in the feed additive composition at more than about 2×10⁵ CFU/g composition, suitably more than about 2×10⁶ CFU/g composition, suitably more than about 3.75×10⁷ CFU/g composition.

Any of the feed additives described herein may also comprise in addition to the GH 30 glucuronoxylanases and GH10 xylanases described herein used either alone or (a) in combination with at least one direct fed microbial or (b) in combination with at least one other enzyme or (c) in combination with at least one direct fed microbial and at least one other enzyme, and (d) at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, magnesium formate, magnesium sorbate, sodium metabisulfite, methyl paraben and propyl paraben.

In still another aspect, there is disclosed a granulated feed additive composition for use in animal feed comprising a at least one polypeptide having xylanase activity as described herein, used either alone or in combination with at least one direct fed microbial or in combination with at least one other enzyme or in combination with at least one direct fed microbial and at least one other enzyme, wherein the granulated feed additive composition comprises particles produced by a process selected from the group consisting of high shear granulation, drum granulation, extrusion, spheronization, fluidized bed agglomeration, fluidized bed spray coating, spray drying, freeze drying, prilling, spray chilling, spinning disk atomization, coacervation, tableting, or any combination of the above processes.

Furthermore, the particles of the granulated feed additive composition can have a mean diameter of greater than 50 microns and less than 2000 microns

The feed additive composition can be a liquid form and the liquid form can also be said suitable for spray-drying on a feed pellet.

Animal feeds may include plant material such as corn, wheat, sorghum, soybean, canola, sunflower or mixtures of any of these plant materials or plant protein sources for poultry, pigs, ruminants, aquaculture and pets. The animal feeds of interest herein are cereal-based animal feeds comprising corn or rice. It is contemplated that animal performance parameters, such as growth, feed intake and feed efficiency, but also improved uniformity, reduced ammonia concentration in the animal house and consequently improved welfare and health status of the animals will be improved. More specifically, as used herein, “animal performance” may be determined by the feed efficiency and/or weight gain of the animal and/or by the feed conversion ratio and/or by the digestibility of a nutrient in a feed (e.g. amino acid digestibility) and/or digestible energy or metabolizable energy in a feed and/or by nitrogen retention and/or by the ability of an animal to avoid the negative effects of necrotic enteritis and/or by the immune response of the subject.

Preferably “animal performance” is determined by feed efficiency and/or weight gain of the animal and/or by the feed conversion ratio.

By “improved animal performance” it is meant that there is increased feed efficiency, and/or increased weight gain and/or reduced feed conversion ratio and/or improved digestibility of nutrients or energy in a feed and/or by improved nitrogen retention and/or by improved ability to avoid the negative effects of necrotic enteritis and/or by an improved immune response in the subject resulting from the use of feed additive composition of the present invention in feed in comparison to feed which does not comprise said feed additive composition.

Preferably, by “improved animal performance” it is meant that there is increased feed efficiency and/or increased weight gain and/or reduced feed conversion ratio. As used herein, the term “feed efficiency” refers to the amount of weight gain in an animal that occurs when the animal is fed ad-libitum or a specified amount of food during a period of time.

By “increased feed efficiency” it is meant that the use of a feed additive composition according the present invention in feed results in an increased weight gain per unit of feed intake compared with an animal fed without said feed additive composition being present.

As used herein, the term “feed conversion ratio” refers to the amount of feed fed to an animal to increase the weight of the animal by a specified amount.

An improved feed conversion ratio means a lower feed conversion ratio.

By “lower feed conversion ratio” or “improved feed conversion ratio” it is meant that the use of a feed additive composition in feed results in a lower amount of feed being required to be fed to an animal to increase the weight of the animal by a specified amount compared to the amount of feed required to increase the weight of the animal by the same amount when the feed does not comprise said feed additive composition.

Nutrient digestibility as used herein means the fraction of a nutrient that disappears from the gastro-intestinal tract or a specified segment of the gastro-intestinal tract, e.g. the small intestine. Nutrient digestibility may be measured as the difference between what is administered to the subject and what comes out in the faeces of the subject, or between what is administered to the subject and what remains in the digesta on a specified segment of the gastro intestinal tract, e.g. the ileum.

Nutrient digestibility as used herein may be measured by the difference between the intake of a nutrient and the excreted nutrient by means of the total collection of excreta during a period of time; or with the use of an inert marker that is not absorbed by the animal, and allows the researcher calculating the amount of nutrient that disappeared in the entire gastro-intestinal tract or a segment of the gastro-intestinal tract. Such an inert marker may be titanium dioxide, chromic oxide or acid insoluble ash. Digestibility may be expressed as a percentage of the nutrient in the feed, or as mass units of digestible nutrient per mass units of nutrient in the feed.

Nutrient digestibility as used herein encompasses starch digestibility, fat digestibility, protein digestibility, and amino acid digestibility.

Energy digestibility as used herein means the gross energy of the feed consumed minus the gross energy of the faeces or the gross energy of the feed consumed minus the gross energy of the remaining digesta on a specified segment of the gastro-intestinal tract of the animal, e.g. the ileum. Metabolizable energy as used herein refers to apparent metabolizable energy and means the gross energy of the feed consumed minus the gross energy contained in the faeces, urine, and gaseous products of digestion. Energy digestibility and metabolizable energy may be measured as the difference between the intake of gross energy and the gross energy excreted in the faeces or the digesta present in specified segment of the gastro-intestinal tract using the same methods to measure the digestibility of nutrients, with appropriate corrections for nitrogen excretion to calculate metabolizable energy of feed.

In some embodiments, the compositions described herein can improve the digestibility or utilization of dietary hemicellulose or fibre in a subject. In some embodiments, the subject is a pig.

Nitrogen retention as used herein means as subject's ability to retain nitrogen from the diet as body mass. A negative nitrogen balance occurs when the excretion of nitrogen exceeds the daily intake and is often seen when the muscle is being lost. A positive nitrogen balance is often associated with muscle growth, particularly in growing animals.

Nitrogen retention may be measured as the difference between the intake of nitrogen and the excreted nitrogen by means of the total collection of excreta and urine during a period of time. It is understood that excreted nitrogen includes undigested protein from the feed, endogenous proteinaceous secretions, microbial protein, and urinary nitrogen.

The term survival as used herein means the number of subject remaining alive. The term “improved survival” may be another way of saying “reduced mortality”.

The term carcass yield as used herein means the amount of carcass as a proportion of the live body weight, after a commercial or experimental process of slaughter. The term carcass means the body of an animal that has been slaughtered for food, with the head, entrails, part of the limbs, and feathers or skin removed. The term meat yield as used herein means the amount of edible meat as a proportion of the live body weight, or the amount of a specified meat cut as a proportion of the live body weight.

An “increased weight gain” refers to an animal having increased body weight on being fed feed comprising a feed additive composition compared with an animal being fed a feed without said feed additive composition being present.

In the present context, it is intended that the term “pet food” is understood to mean a food for a household animal such as, but not limited to, dogs, cats, gerbils, hamsters, chinchillas, fancy rats, guinea pigs; avian pets, such as canaries, parakeets, and parrots; reptile pets, such as turtles, lizards and snakes; and aquatic pets, such as tropical fish and frogs.

In another embodiment, there is disclosed a corn-based animal feed comprising at least one GH30 enzyme with glucuronoxylanase activity and at least one GH10 enzyme having endo-beta-1,4-xylanase activity wherein the combination is better in stimulating growth of beneficial bacteria in a digestive tract of a monogastric animal when compared to the use of the GH10 xylanase alone.

There is also disclosed a corn-based animal feed comprising at least one GH30 enzyme with glucuronoxylanase activity and at least one GH10 enzyme having endo-beta-1,4-xylanase activity wherein said combination is capable of increasing production of at least one short chain fatty acid in a monogastric animal when compared to the use of GH10 alone.

The short chain fatty acid can be selected from the group consisting of acetic acid, propionic acid and butyric acid.

This animal feed may further comprise at least one DFM or at least on other enzyme or a combination of both at least one DFM and one or more other enzymes as has already been described herein.

The terms “animal feed composition,” “feed”, “feedstuff” and “fodder” are used interchangeably and can comprise one or more feed materials selected from the group comprising a) cereals, such as small grains (e.g., wheat, barley, rye, oats and combinations thereof) and/or large grains such as maize or sorghum; b) by products from cereals, such as corn gluten meal, Distillers Dried Grains with Solubles (DDGS) (particularly corn based Distillers Dried Grains with Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; and/or e) minerals and vitamins.

The term “cereal” is used to describe any grass cultivated for the edible components of its grain (botanically, a type of fruit called a caryopsis), composed of the endosperm, germ, and bran. Cereal grains such as corn and rice are grown in greater quantities and provide more food energy worldwide than any other type of crop and are therefore staple crops.

The terms “feed additive”, “feed additive composition” and “enzyme composition” are used interchangeably herein.

The feed may be in the form of a solution or as a solid or as a semi-solid depending on the use and/or the mode of application and/or the mode of administration.

When used as, or in the preparation of, a feed, such as functional feed, the enzyme or feed additive composition described herein may be used in conjunction with one or more of: a nutritionally acceptable carrier, a nutritionally acceptable diluent, a nutritionally acceptable excipient, a nutritionally acceptable adjuvant, a nutritionally active ingredient. For example, there be mentioned at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, magnesium formate, magnesium sorbate, sodium metabisulfite, methyl paraben and propyl paraben.

In another aspect, the feed additive disclosed herein is admixed with a feed component to form a feedstuff. The term “feed component” as used herein means all or part of the feedstuff. Part of the feedstuff may mean one constituent of the feedstuff or more than one constituent of the feedstuff, e.g. 2 or 3 or 4 or more. In one embodiment, the term “feed component” encompasses a premix or premix constituents. Preferably, the feed may be a fodder, or a premix thereof, a compound feed, or a premix thereof. A feed additive composition may be admixed with a compound feed, a compound feed component or to a premix of a compound feed or to a fodder, a fodder component, or a premix of a fodder.

Any feedstuff described herein may comprise one or more feed materials selected from the group comprising a) cereals, such as small grains (e.g., wheat, barley, rye, oats, triticale and combinations thereof) and/or large grains such as maize or sorghum; b) by products from cereals, such as corn gluten meal, wet-cake (particularly corn based wet-cake), Distillers Dried Grains (DDG) (particularly corn based Distillers Dried Grains (cDDG)), Distillers Dried Grains with Solubles (DDGS) (particularly corn based Distillers Dried Grains with Solubles (cDDGS)), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; e) minerals and vitamins.

The term “fodder” as used herein means any food which is provided to an animal (rather than the animal having to forage for it themselves). Fodder encompasses plants that have been cut. Furthermore, fodder includes silage, compressed and pelleted feeds, oils and mixed rations, and also sprouted grains and legumes.

Fodder may be obtained from one or more of the plants selected from: corn (maize), alfalfa (Lucerne), barley, birdsfoot trefoil, brassicas, Chau moellier, kale, rapeseed (canola), rutabaga (swede), turnip, clover, alsike clover, red clover, subterranean clover, white clover, fescue, brome, millet, oats, sorghum, soybeans, trees (pollard tree shoots for tree-hay), wheat, and legumes.

The term “compound feed” means a commercial feed in the form of a meal, a pellet, nuts, cake or a crumble. Compound feeds may be blended from various raw materials and additives. These blends are formulated according to the specific requirements of the target animal.

Compound feeds can be complete feeds that provide all the daily required nutrients, concentrates that provide a part of the ration (protein, energy) or supplements that only provide additional micronutrients, such as minerals and vitamins.

The main ingredients used in compound feed are the feed grains, which include corn, wheat, canola meal, rapeseed meal, lupin, soybeans, sorghum, oats, and barley.

Suitably a premix as referred to herein may be a composition composed of microingredients such as vitamins, minerals, chemical preservatives, antibiotics, fermentation products, and other essential ingredients. Premixes are usually compositions suitable for blending into commercial rations.

In one embodiment the feedstuff comprises or consists of corn, DDGS (such as cDDGS), wheat, wheat bran or any combination thereof.

In one embodiment the feed component may be corn, DDGS (e.g. cDDGS), wheat, wheat bran or a combination thereof. In one embodiment the feedstuff comprises or consists of corn, DDGS (such as cDDGS) or a combination thereof.

A feedstuff described herein may contain at least 30%, at least 40%, at least 50% or at least 60% by weight corn and soybean meal or corn and full fat soy, or wheat meal or sunflower meal.

For example, a feedstuff may contain between about 5 to about 40% corn DDGS. For poultry, the feedstuff on average may contain between about 7 to 15% corn DDGS. For swine (pigs), the feedstuff may contain on average 5 to 40% corn DDGS. It may also contain corn as a single grain, in which case the feedstuff may comprise between about 35% to about 80% corn.

In feedstuffs comprising mixed grains, e.g. comprising corn and wheat for example, the feedstuff may comprise at least 10% corn.

In addition, or in the alternative, a feedstuff also may comprise at least one high fibre feed material and/or at least one by-product of the at least one high fibre feed material to provide a high fibre feedstuff. Examples of high fibre feed materials include: wheat, barley, rye, oats, by products from cereals, such as corn gluten meal, corn gluten feed, wet-cake, Distillers Dried Grains (DDG), Distillers Dried Grains with Solubles (DDGS), wheat bran, wheat middlings, wheat shorts, rice bran, rice hulls, oat hulls, palm kernel, and citrus pulp. Some protein sources may also be regarded as high fibre: protein obtained from sources such as sunflower, lupin, fava beans and cotton. In one aspect, the feedstuff as described herein comprises at least one high fibre material and/or at least one by-product of the at least one high fibre feed material selected from the group consisting of Distillers Dried Grains with Solubles (DDGS), particularly cDDGS, wet-cake, Distillers Dried Grains (DDG), particularly cDDG, wheat bran, and wheat for example. In one embodiment the feedstuff of the present invention comprises at least one high fibre material and/or at least one by-product of the at least one high fibre feed material selected from the group consisting of Distillers Dried Grains with Solubles (DDGS), particularly cDDGS, wheat bran, and wheat for example.

The feed may be one or more of the following: a compound feed and premix, including pellets, nuts or (cattle) cake; a crop or crop residue: corn, soybeans, sorghum, oats, barley copra, straw, chaff, sugar beet waste; fish meal; meat and bone meal; molasses; oil cake and press cake; oligosaccharides; conserved forage plants: silage; seaweed; seeds and grains, either whole or prepared by crushing, milling etc.; sprouted grains and legumes; yeast extract.

The term “feed” as used herein encompasses in some embodiments pet food. A pet food is plant or animal material intended for consumption by pets, such as dog food or cat food. Pet food, such as dog and cat food, may be either in a dry form, such as kibble for dogs, or wet canned form. Cat food may contain the amino acid taurine.

Animal feed can also include a fish food. A fish food normally contains macro nutrients, trace elements and vitamins necessary to keep captive fish in good health. Fish food may be in the form of a flake, pellet or tablet. Pelleted forms, some of which sink rapidly, are often used for larger fish or bottom feeding species. Some fish foods also contain additives, such as beta carotene or sex hormones, to artificially enhance the color of ornamental fish.

In still another aspect, animal feed encompasses bird food. Bird food includes food that is used both in birdfeeders and to feed pet birds. Typically, bird food comprises of a variety of seeds, but may also encompass suet (beef or mutton fat).

As used herein the term “contacted” refers to the indirect or direct application of a xylanase enzyme (or composition comprising xylanase) to a product (e.g. the feed). Examples of application methods which may be used, include, but are not limited to, treating the product in a material comprising the feed additive composition, direct application by mixing the feed additive composition with the product, spraying the feed additive composition onto the product surface or dipping the product into a preparation of the feed additive xylanase composition. In one embodiment the feed additive composition of the present invention is preferably admixed with the product (e.g. feedstuff). Alternatively, the feed additive composition may be included in the emulsion or raw ingredients of a feedstuff. For some applications, it is important that the composition is made available on or to the surface of a product to be affected/treated. This allows the composition to impart a performance benefit.

In some aspects, the feed additives described are used for the pre-treatment of food or feed. For example, the feed having 10-300% moisture is mixed and incubated with the xylanases at 5-80° C., preferably at 25-50° C., more preferably between 30-45° C. for 1 min to 72 hours under aerobic conditions or 1 day to 2 months under anaerobic conditions. The pre-treated material can be fed directly to the animals (so called liquid feeding). The pre-treated material can also be steam pelleted at elevated temperatures of 60-120° C. The xylanases can be impregnated to feed or food material by a vacuum coater.

Such feed additives may be applied to intersperse, coat and/or impregnate a product (e.g. feedstuff or raw ingredients of a feedstuff) with a controlled amount of one or more enzymes.

Preferably, the feed additive composition will be thermally stable to heat treatment up to about 70° C.; up to about 85° C.; or up to about 95° C. The heat treatment may be performed for up to about 1 minute; up to about 5 minutes; up to about 10 minutes; up to about 30 minutes; up to about 60 minutes. The term thermally stable means that at least about 75% of the enzyme components and/or DFM that were present/active in the additive before heating to the specified temperature are still present/active after it cools to room temperature. Preferably, at least about 80% of the xylanase component and/or DFM comprising one or more bacterial strains that were present and active in the additive before heating to the specified temperature are still present and active after it cools to room temperature. In a particularly preferred embodiment the feed additive is homogenized to produce a powder.

Alternatively, the feed additive is formulated to granules as described in WO2007/044968 (referred to as TPT granules) incorporated herein by reference.

In another preferred embodiment when the feed additive is formulated into granules the granules comprise a hydrated barrier salt coated over the protein core. The advantage of such salt coating is improved thermo-tolerance, improved storage stability and protection against other feed additives otherwise having adverse effect on the at least one xylanase and/or DFM comprising one or more bacterial strains. Preferably, the salt used for the salt coating has a water activity greater than 0.25 or constant humidity greater than 60% at 20° C. Preferably, the salt coating comprises a Na₂SO₄.

The method of preparing a feed additive may also comprise the further step of pelleting the powder. The powder may be mixed with other components known in the art. The powder, or mixture comprising the powder, may be forced through a die and the resulting strands are cut into suitable pellets of variable length.

Optionally, the pelleting step may include a steam treatment, or conditioning stage, prior to formation of the pellets. The mixture comprising the powder may be placed in a conditioner, e.g. a mixer with steam injection. The mixture is heated in the conditioner up to a specified temperature, such as from 60-100° C., typical temperatures would be 70° C., 80° C., 85° C., 90° C. or 95° C. The residence time can be variable from seconds to minutes and even hours. Such as 5 seconds, 10 seconds, 15 seconds, 30 seconds, 1 minutes 2 minutes, 5 minutes, 10 minutes, 15 minutes, 30 minutes and 1 hour. It will be understood that the xylanases (or composition comprising the xylanases) described herein are suitable for addition to any appropriate feed material.

It will be understood by the skilled person that different animals require different feedstuffs, and even the same animal may require different feedstuffs, depending upon the purpose for which the animal is reared.

Optionally, the feedstuff may also contain additional minerals such as, for example, calcium and/or additional vitamins. In some embodiments, the feedstuff is a corn soybean meal mix.

Feedstuff is typically produced in feed mills in which raw materials are first ground to a suitable particle size and then mixed with appropriate additives. The feedstuff may then be produced as a mash or pellets; the later typically involves a method by which the temperature is raised to a target level and then the feed is passed through a die to produce pellets of a particular size. The pellets are allowed to cool. Subsequently liquid additives such as fat and enzyme may be added. Production of feedstuff may also involve an additional step that includes extrusion or expansion prior to pelleting, in particular by suitable techniques that may include at least the use of steam.

The feed additive and/or the feedstuff comprising the feed additive may be used in any suitable form. The feed additive composition may be used in the form of solid or liquid preparations or alternatives thereof. Examples of solid preparations include powders, pastes, boluses, capsules, pellets, tablets, dusts, and granules which may be wettable, spray-dried or freeze-dried. Examples of liquid preparations include, but are not limited to, aqueous, organic or aqueous-organic solutions, suspensions and emulsions.

In some applications, the feed additive may be mixed with feed or administered in the drinking water.

A feed additive as taught herein with a feed acceptable carrier, diluent or excipient, and (optionally) packaging.

The feedstuff and/or feed additive may be combined with at least one mineral and/or at least one vitamin. The compositions thus derived may be referred to herein as a premix.

The xylanases and the glucuronoxylanases can be present in the feedstuff in the range of 1 ppb (parts per billion) to 10% (w/w) based on pure enzyme protein. In some embodiments, the xylanase is present in the feedstuff is in the range of 0.1-100 ppm (parts per million). A preferred dose can be 0.2-20 g of xylanase per ton of feed product or feed composition or a final dose of 0.2-20 ppm xylanase in final product.

Preferably, the xylanases present in the feedstuff should be at least about 250 XU/kg or at least about 500 XU/kg feed, at least about 750 XU/kg feed, or at least about 1000 XU/kg feed, or at least about 1500 XU/kg feed, or at least about 2000 XU/kg feed or at least about 2500 XU/kg feed, or at least about 3000 XU/kg feed, or at least about 3500 XU/kg feed, or at least about 4000 XU/kg feed.

In another aspect, the xylanases as described herein can be present in the feedstuff at less than about 30,000 XU/kg feed, or at less than about 20,000 XU/kg feed, or at less than about 10,000 XU/kg feed, or at less than about 8000 XU/kg feed, or at less than about 6000 XU/kg feed, or at less than about 5000 XU/kg feed.

Ranges can include, but are not limited to, any combination of the lower and upper ranges discussed above.

The xylanase activity can be expressed in xylanase units (XU) measured at pH 5.0 with AZCL-arabinoxylan (azurine-crosslinked wheat arabinoxylan, Xylazyme tablets, Megazyme) as substrate. Hydrolysis by endo-(1-4)-ß-D-xylanase (xylanase) produces water soluble dyed fragments, and the rate of release of these (increase in absorbance at 590 nm) can be related directly to enzyme activity. The xylanase units (XU) are determined relatively to an enzyme standard (Danisco Xylanase, available from Danisco Animal Nutrition) at standard reaction conditions, which are 40° C., 10 min reaction time in McIlvaine buffer, pH 5.0.

The xylanase activity of the standard enzyme is determined as amount of released reducing sugar end groups from an oat-spelt-xylan substrate per min at pH 5.3 and 50° C. The reducing sugar end groups react with 3,5-Dinitrosalicylic acid and formation of the reaction product can be measured as increase in absorbance at 540 nm. The enzyme activity is quantified relative to a xylose standard curve (reducing sugar equivalents). One xylanase unit (XU) is the amount of standard enzyme that releases 0.5 μmol of reducing sugar equivalents per min at pH 5.3 and 50° C.

Non-limiting examples of compositions and methods disclosed herein include:

1. An additive for animal feed comprising corn or rice, said feed additive comprising at least one enzyme with glucuronoxylanase activity and at least one enzyme having endo-beta-1,4-xylanase activity wherein degradation of insoluble glucuronoxylan is greater than if either enzyme was used alone.
2. The feed additive of embodiment 1 wherein the xylanase having glucuronoxylanase activity is derived from Bacillus or Paenibacillus sp.
3. The feed additive of embodiment 1 wherein the xylanase having endo-beta-1,4-xylanase activity is derived from Fusarium sp.
4. The feed additive of embodiment 1 wherein at least one of the xylanases is recombinantly produced.
5. A feed additive comprising at least one enzyme with glucuronoxylanase activity and at least one enzyme having endo-beta-1,4-xylanase activity wherein said combination is better in stimulating growth of beneficial bacteria in a digestive tract of a monogastric animal fed a corn based diet when compared to the use of the xylanase having endo-beta-1,4-xylanase activity alone.
6. A feed additive comprising at least one enzyme with glucuronoxylanase activity and at least one enzyme having endo-beta-1,4-xylanase activity wherein said combination is capable of increasing production of at least one short chain fatty acid in a monogastric animal fed a corn based diet when compared to the use of the xylanase having endo-beta-1,4-xylanase activity alone.
7. The feed additive of embodiment 6 wherein the short chain fatty acid is selected from the group consisting of acetic acid, propionic acid or butyric acid.
8. The feed additive of any embodiment 1-7 which further comprises one or more of the enzymes selected the group consisting of an amylase, protease, endo-glucanase and phytase.
9. A premix comprising the feed additive of any embodiments 1-7 and at least one vitamin and/or mineral.
10. A corn or rice-based animal feed comprising at least one enzyme with glucuronoxylanase activity and at least one enzyme having endo-beta-1,4-xylanase activity wherein degradation of insoluble glucuronoxylan is greater than if either enzyme was used alone.
11. A corn-based animal feed comprising at least one enzyme with glucuronoxylanase activity and at least one enzyme having endo-beta-1,4-xylanase activity wherein said combination is better in stimulating growth of beneficial bacteria in a digestive tract of a monogastric animal when compared to the use of the xylanase having avalone.
12. A corn-based animal feed comprising at least one enzyme with glucuronoxylanase activity and at least one enzyme having endo-beta-1,4-xylanase activity wherein said combination is capable of increasing production of at least one short chain fatty acid in a monogastric animal when compared to the use of the xylanase having endo-beta-1,4-xylanase activity alone.
13. The animal feed of embodiment 12 wherein the short chain fatty acid is selected from the group consisting of acetic acid, propionic acid or butyric acid.
14. The animal feed of any of embodiments 11-13 which further comprises one or more of the enzymes selected the group consisting of an amylase, protease, endo-glucanase and phytase.

### EXAMPLES

Unless defined otherwise herein, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used with this disclosure.

The disclosure is further defined in the following Examples. It should be understood that the Examples, while indicating certain embodiments, is given by way of illustration only. From the above discussion and the Examples, one skilled in the art can ascertain essential characteristics of this disclosure, and without departing from the spirit and scope thereof, can make various changes and modifications to adapt to various uses and conditions.

### Example 1

### Assays

Protein determination. The concentrations of purified protein samples were measured in NanoDrop 2000 Spectrophotometer (Thermo Fisher Scientific Inc.) using A280 method according to the instructions of the manufacturer. The extinction coefficient (0.1%) of each protein was used for protein concentration calculation. The extinction coefficient (0.1%) for BsuGH30 and BliXyn1 is 2.1, and respectively 1.8 and 1.9 for FveXyn4 and FveXyn4.v1.
Xylanase activity assay. 1% (w/w) substrate solution: 0.2 g of 4-O-Methyl-D-glucurono-D-xylan dyed with Remazol brilliant blue R (RBB-Xylan) (Sigma catalog number 66960) was mixed with 100 mM phosphate buffer, pH 6.0 and brought to boil with stirring until the powder dissolves. After cooling to room temperature, the final weight of the solution was adjusted to 20 g.
In a test tube 500 μL enzyme solution was mixed with 500 μL 1% (w/w) substrate solution. The mixture was incubated 30 minutes at 50° C. The reaction was stopped and high molecular weight fragments were precipitated with addition of 5 mL 96% ethanol and subsequent mixing. The tubes were left to stand at room temperature for 10 min, before repeated mixing and centrifugation at 1500×g for 10 min at 20° C. The response was measured as the difference in the absorbance at 585 nm and 445 nm for the supernatants.
Degradation of water-unextractable arabinoxylan (WU-AX) measured as increase in extractable arabinoxylan upon xylanase treatment. 5% or 10% substrate solution: corn DDGS or rice bran ground to a particle size <212 μm was hydrated in 100 mM MES buffer, pH 6.0 by stirring 15 min at 600 rpm. Subsequently, pH was adjusted to pH 6.0. 190 μL/well substrate solution was transferred to the substrate plates, which were stored at −20° C. until use.
All dilutions were prepared with a Biomek dispensing robot (Beckman Coulter, USA) in 96 well plates (substrate plate and collection plate: Clear Polystyrene Microplate, Corning, Cat. no. 9017; Filter plate: 0.2 μm PVDF membrane, Corning, Cat. no. 3504). All enzymes were diluted with dilution buffer (50 mM sodium acetate buffer, pH 5.0). 10 μL solution was added to the premade substrate plates. For the blank samples, 10 μL dilution buffer was added, for test of single enzymes 10 μL enzyme solution or 5 μL enzyme solution and 5 μL dilution buffer was added, and for test of combinations 5 μL of each enzyme solution was added. The plates were incubated at 40° C. for 120 minutes in an iEMS microplate incubator (Thermo Scientific). After end incubation, the sample was transferred to a filter plate, which was placed on top of a collection plates and centrifuged for 10 min at 1666×g. The collection plates were stored at −20° C. and subsequently diluted 10 times with DI water prior to further analysis.
The total amount of C5 sugar units in solution was measured as xylose equivalents by the Douglas method using a continuous flow injection apparatus (SKALAR Analytical, Breda, The Netherlands) as described by Rouau X & Surget A (1994). The combination of heat and low pH will lead to a decomposition of arabinoxylan into the pentose mono-sugars, arabinose and xylose, which will further dehydrate into furfural. By reaction with phloroglucinol a colored complex is formed.
Essentially, the filtered samples were treated at 95° C. with a 55:1 mixture of CH₃COOH and HCl and a 20% solution of phloroglucinol (1,3,5-trihydroxybenzene, Merck catalog number 107069) dissolved in ethanol. By measuring the absorbance at 550 nm with 510 nm as reference wavelength, the concentration of pentose mono-sugars in solution was measured as xylose equivalents using a xylose standard curve (5-300 μg xylose/mL). Unlike the pentose-phloroglucinol complex, the absorbance of the hexose-phloroglucinol complex is constant at these wavelengths.
The extracted arabinoxylan was determined as the mass of the hydrated xylose equivalents (molar mass: 150.13 g/mol) per substrate mass (cDDGS or rice bran). The results are reported as the increase in extractable arabinoxylan calculated as the difference between extracted arabinoxylan for the enzyme treated sample and for the blank sample.

### Calculation:

Enzyme inclusion rates (μg/g)=Volume of enzyme sample (μL)×concentration of enzyme sample (μg/mL)/(190 μL×substrate concentration (g/mL))
Extracted arabinoxylan (mg/g)=Concentration of xylose equivalents (mg/mL)×200 μL/(190 μL×substrate concentration (g/mL))
Increase in extractable arabinoxylan (mg/g)=Extracted arabinoxylan (mg/g), enzyme treated sample—Extracted arabinoxylan (mg/g), blank sample
   Performance after pepsin exposure: The enzyme sample was diluted to a final concentration of 2 μg/mL with solution A (100 mM glycine buffer, pH 3.5 containing 0.2 mg/mL pepsin) or as control in solution B (50 mM sodium acetate buffer, pH 5.0) and incubated for 2 hours at 40° C. with shaking in an iEMS shaker (Thermo Scientific). After end incubation, the performance of the pepsin treated sample (diluted in solution A) was compared to the control sample (diluted in solution B) using the WU-AX degradation assay described above.

### Example 2

### Identification of GH30 Glucuronoxylanases

Three GH30 glucuronoxylanases: BsuGH30 (also known as XynC), BliXyn1, and BamGh2 were identified from the NCBI database (Accession numbers are WP_063694996.1, WP_035400315.1, and ABS74177, respectively). In addition, homologues of these glucuronoxylanases were identified by sequencing the genomes of Bacillus safensis, Paenibacillus macerans, Paenibacillus cookii DSM 16944, and Paenibacillus tundrae DSM 21291 strains. The entire genomes of these organisms were sequenced using Illumina's next generation sequencing technology, assembled, and the contigs were annotated. The donor organism origin, protein name, and SEQ ID numbers for the genes and native proteins are listed in Table 3.

TABLE 3
GH30 glucuronoxylanases

| | | | Full length | Mature |
|---|---|---|---|---|
| Origin | Name | Gene | Protein | Protein |
| Bacillus subtilis | BsuGH30 | SEQ ID No. 1 | SEQ ID No. 2 | SEQ ID No. 29 |
| Bacillus licheniformis | BliXyn1 | SEQ ID No. 3 | SEQ ID No. 4 | SEQ ID No. 30 |
| Bacillus amyloliquefaciens FZB42 | BamGh2 | SEQ ID No. 5 | SEQ ID No. 6 | SEQ ID No. 31 |
| Bacillus safensis | BsaXyn1 | SEQ ID No. 7 | SEQ ID No. 8 | SEQ ID No. 32 |
| Paenibacillus macerans | PmaXyn4 | SEQ ID No. 9 | SEQ ID No. 10 | SEQ ID No. 33 |
| Paenibacillus cookii DSM 16944 | PcoXyn1 | SEQ ID No. 11 | SEQ ID No. 12 | SEQ ID No. 34 |
| Paenibacillus tundrae DSM 21291 | PtuXyn2 | SEQ ID No. 13 | SEQ ID No. 14 | SEQ ID No. 35 |

### Example 3

### Cloning and Expression of GH30 Glucuronoxylanases

Synthetic genes encoding seven homologous glucuronoxylanase genes described in Example 2 (Table 1) were generated using techniques known in the art and inserted into the expression vector p2JM103BBI (Vogtentanz, Protein Expr Purif, 55:40-52, 2007). The resulting expression plasmids contain: an aprE promoter (SEQ ID No 43), an aprE signal sequence (SEQ ID No. 44 represents the amino acid sequence), an oligonucleotide that encodes the tripeptide Ala-Gly-Lys at the 5′ end, the synthetic nucleotide sequence encoding the mature region of the glucuronoxylanase gene of interest (SEQ ID No. 15, 17, 19, 21, 23, 25 or 27) and the AprE terminator (SEQ ID No 45). Table 4 provides the sequence listing numbers of each recombinant gene used for GH30 expression and the resulting full length and mature protein sequences.

TABLE 4
SEQ ID numbers of GH30 glucuronoxylanases (synthetic genes and
recombinant protein sequences)

| | | Full length | Mature |
|---|---|---|---|
| Origin | Synthetic Gene | Recombinant Protein | Recombinant Protein |
| Bacillus subtilis | SEQ ID No. 15 | SEQ ID No. 16 | SEQ ID No. 36 |
| Bacillus licheniformis | SEQ ID No. 17 | SEQ ID No. 18 | SEQ ID No. 37 |
| Bacillus amyloliquefaciens FZB42 | SEQ ID No. 19 | SEQ ID No. 20 | SEQ ID No. 38 |
| Bacillus safensis | SEQ ID No. 21 | SEQ ID No. 22 | SEQ ID No. 39 |
| Paenibacillus macerans | SEQ ID No. 23 | SEQ ID No. 24 | SEQ ID No. 40 |
| Paenibacillus cookii DSM 16944 | SEQ ID No. 25 | SEQ ID No. 26 | SEQ ID No. 41 |
| Paenibacillus tundrae DSM 21291 | SEQ ID No. 27 | SEQ ID No. 28 | SEQ ID No. 42 |

A suitable B. subtilis host strain was transformed with each of the expression plasmids and the transformed cells were spread on Luria Agar plates supplemented with 5 ppm chloramphenicol. To produce each of the enzymes listed above, B. subtilis transformants containing the plasmids were grown in 250 mL shake flasks in a MOPS based defined medium, supplemented with additional 5 mM CaCl₂.

### Example 4

### Purification of Glucuronoxylanases

BsuGH30 was purified in three chromatographic steps. The clarified culture supernatant, equilibrated to 20 mM sodium phosphate pH 6.0 was first loaded on an SP cation exchange column, eluted with a salt (NaCl) gradient. Fractions containing protein of interest were adjusted to 1M ammonium sulfate prior to loading on a HiLoad phenyl-HP Sepharose column and eluted with a gradient of 1M-0 ammonium sulfate in 20 mM Tris pH 7.0. Fractions containing protein of interest were then loaded on a Superdex 75 column and eluted with 20 mM sodium phosphate pH 7.0 with 0.15 M NaCl.

BliXyn1 and BsaXyn1 enzymes were purified in two chromatographic steps. The clarified culture supernatant was concentrated and equilibrated to 0.8 M of ammonium sulfate prior to loading onto a Phenyl Sepharose HP column. Fractions containing protein of interest were eluted with 20 mM Tris-HCl, pH 7.5, pooled, concentrated and loaded onto a Superdex 75 column and eluted with 20 mM Tris-HCl pH 7.5 containing 0.15 M NaCl.

BamGh2 was purified in two chromatographic steps. The clarified culture supernatant was concentrated, equilibrated with 20 mM sodium phosphate pH 6, loaded onto SP cation exchange column and protein of interest was eluted with a 0-200 mM NaCl gradient. Fractions containing protein of interest were concentrated, loaded onto a Superdex 75 column and eluted with 20 mM sodium phosphate pH 7.0 with 0.15 M NaCl.

PmaXyn4 was purified in three steps. The clarified culture supernatant adjusted to 65% saturation ammonium sulfate to. The precipitate was collected and suspended in 20 mM sodium acetate pH 5 with 1 M ammonium sulfate, loaded onto a HiPrep phenyl-FF Sepharose column and eluted with a 1-0M ammonium sulfate gradient in buffer. Fractions containing protein of interest were pooled, desalted, loaded onto a HiPrep SP-XL Sepharose cation exchange column, and target protein was eluted with a 0-0.5 M NaCl linear gradient.

PcoXyn1 and PtuXyn2 enzymes were purified in two chromatographic steps. The clarified culture supernatants were concentrated and equilibrated with 1M ammonium sulfate prior to loading onto a phenyl-HP Sepharose column. Fractions containing protein of interest were eluted with a gradient of 1-0M ammonium sulfate in 20 mM Tris pH 8.0, fractions pooled and loaded onto a HiPrep Q-XL Sepharose anion exchange. Protein was eluted with a gradient of 0-0.5 M NaCl.

In all cases, the chromatography resins were obtained from column GE Healthcare, and the final column fractions containing the purified target proteins were pooled and concentrated using a 10K Amicon Ultra-15 device. The final products were 90-95% pure (by SDS-PAGE determination), and were adjusted to 40% glycerol and stored at −20° C. or −80° C. until usage.

### Example 5

### Xylanase Activity of BsuGH30 and BliXyn1

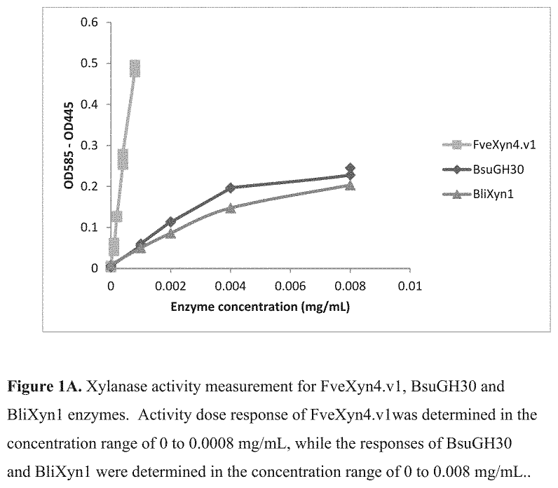

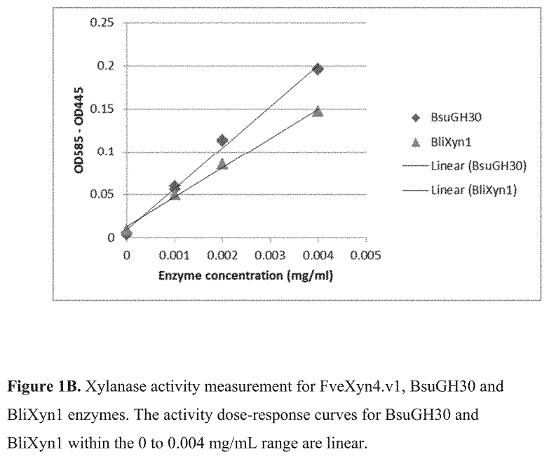

The xylanase activity of BsuGH30, BliXyn1 and the GH10 xylanase FveXyn4.v1 (described in patent application WO2015114112) was determined using soluble 4-O-Methyl-D-glucurono-D-xylan dyed with Remazol brilliant blue R (RBB-Xylan) as substrate. After precipitation of undegraded high molecular weight RBB-Xylan, the absorbance of the supernatant is proportional to the production of low molecular weight fragments by enzyme treatment. Although both BsuGH30 and BliXyn1 exhibited xylanase activity, it is clear from the results presented in FIG. 1, that BsuGH30 and BliXyn1 produced a lower amount of low molecular weight fragments than FveXyn4.v1 at the same enzyme concentration, but also in terms of the maximum amount of low molecular weight fragments obtained with a given substrate concentration. The two GH30 enzymes did not perform as well as the GH10 enzyme in this assay, but BsuGH30 and BliXyn1 were surprisingly good at degrading water unextractable arabinoxylan (WU-AX) from corn as described below.

### Example 6

### Degradation of WU-AX in Corn DDGS by BsuGH30 and BliXyn1

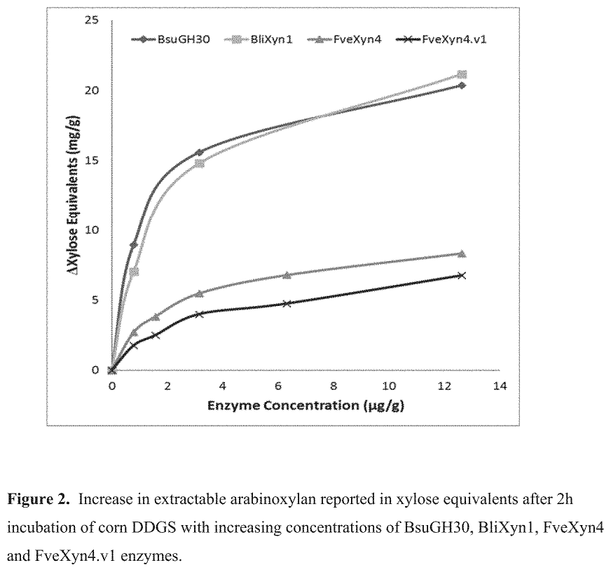

The BsuGH30 and BliXyn1 enzymes were tested together with the GH10 enzymes FveXyn4 (described in patent application WO2014020142) and FveXyn4.v1 (described in patent application WO2015114112) for their ability to degrade the water unextractable arabinoxylans (WU-AX) in corn DDGS using the assay described in Example 1. FIG. 2 shows the increase in extractable arabinoxylan after 2 h incubation of ground corn DDGS with enzyme. The data shows that considerably more arabinoxylan was extractable after incubation with BsuGH30 and BliXyn1 than with the FveXyn4 and FveXyn4.v1 enzymes when tested using the same enzyme concentration. FveXyn4 had previously been shown to be efficient in degrading water unextractable corn DDGS (patent number WO2014020142) but the GH30 enzymes show an even greater ability to degrade water unextractable arabinoxylans in corn DDGS.

### Example 7

### Degradation of WU-AX in Corn DDGS by Additional GH30 Glucuronoxylanases

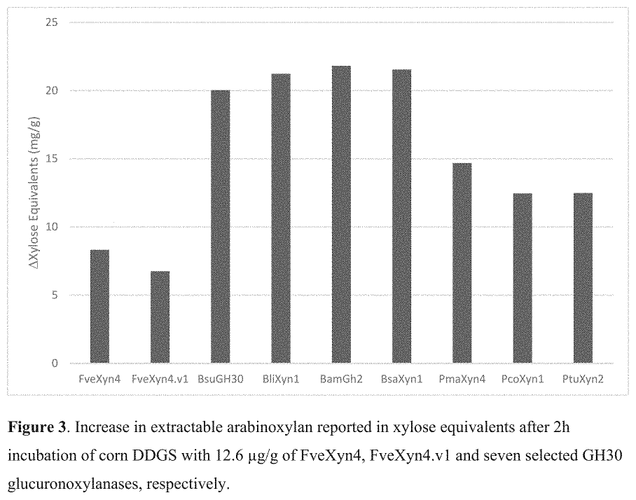

Seven GH30 glucuronoxylanases (BsuGH30, BliXyn1, BamGh2, BsaXyn1, PmaXyn4, PcoXyn1 and PtuXyn2) and two GH10 enzymes (FveXyn4 and FveXyn4.v1) were tested for their ability to degrade water unextractable arabinoxylan in ground corn DDGS using the assay described in Example 1. The seven GH30 glucuronoxylanases and the two GH10 enzymes were tested in increasing concentrations and the results obtained when using 12.6 μg enzyme/g corn DDGS are shown in FIG. 3. The results show that incubation with all tested GH30 glucuronoxylanases resulted in more extractable arabinoxylan than incubation with the GH10 enzymes, FveXyn4 and FveXyn4.v1, when tested at the same doses.

### Example 8

### Degradation of WU-AX in Corn DDGS and Rice Bran by GH30 Glucuronoxylanases in Combination with GH10 Xylanase

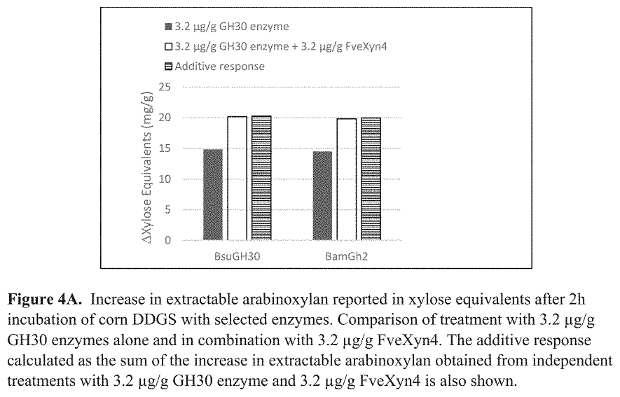

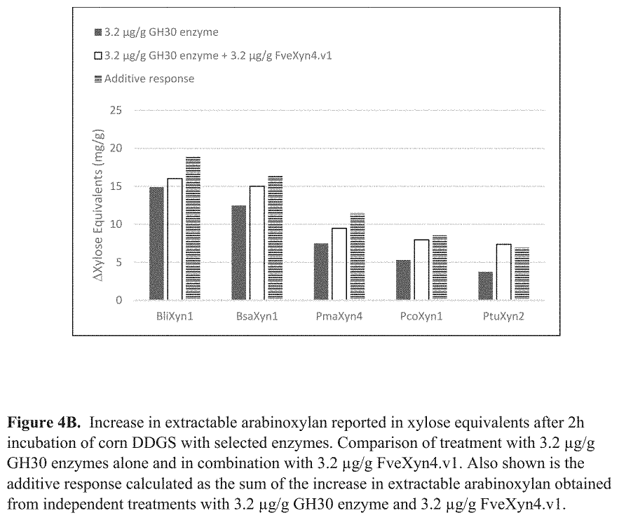

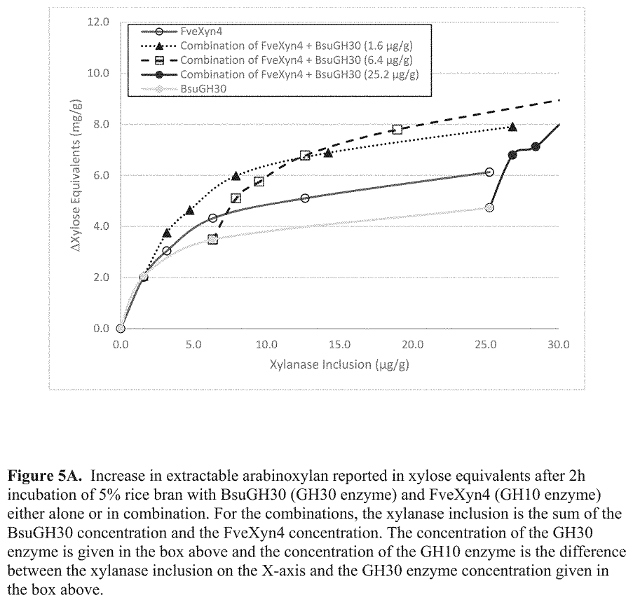

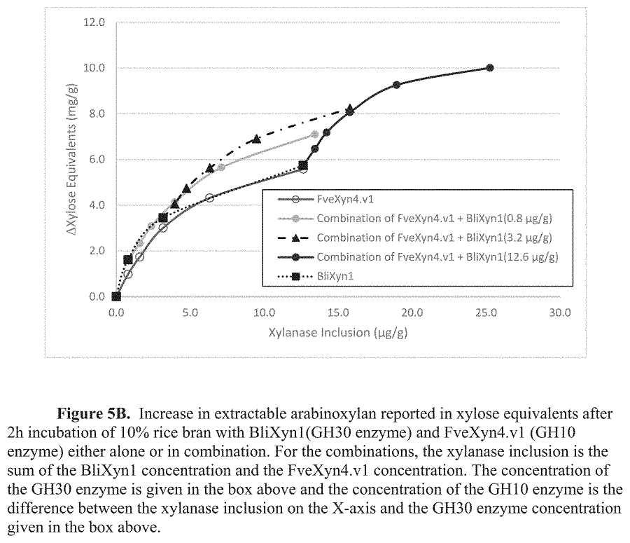

The combination of GH30 glucuronoxylanase with a GH10 xylanase was evaluated using corn DDGS as substrate. FIGS. 5 (A and B) shows the results for GH30 enzymes alone and in combination with the GH10 xylanase FveXyn4 or FveXyn4.v1. Adding the GH10 xylanase to the GH30 enzymes enhanced the increase in extractable arabinoxylan. For BsuGH30, BamGh2, PcoXyn1 and PtuXyn2 the additional increase in the extractable arabinoxylan obtained with the combination of 3.2 μg/g GH30 enzyme plus 3.2 μg/g GH10 xylanase compared to single use of 3.2 μg/g GH30 enzyme equaled the increase obtained with 3.2 μg/g GH10 xylanase alone, and full additivity of the performance of these GH30 enzymes and the tested GH10 enzymes was thereby demonstrated. This is illustrated in the FIG. 4 by comparing the increase obtained with the combination of 3.2 μg/g GH30 enzyme plus 3.2 μg/g GH10 xylanase and the sum of the increase obtained by single use of 3.2 μg/g GH30 enzyme and 3.2 μg/g GH10 xylanase, respectively.

The combination of GH30 glucuronoxylanases and GH10 xylanase was also evaluated using rice bran as substrate. FIG. 5A shows the results for FveXyn4 GH10 and BsuGH30 GH30 enzymes respectively and in combination and FIG. 5B shows the results of FveXyn4.v1 GH10 and BliXyn1 GH30 enzymes respectively and in combination at doses ranging from 0 to 12.6 μg/g rice bran concentration. An increase in extractable arabinoxylan was observed with addition of FveXyn4, FveXyn4.v1, BsuGH30 and BliXyn1, respectively. In all instances, it was found that the combination of GH10 and GH30 enzymes had a synergistic effect, as all tested combinations lead to a greater increase in extractable arabinoxylans than the individual enzymes tested at the same total enzyme concentration; e.g. a comparable increase in extractable arabinoxylan (respectively 7.6 and 7.4 mg/g) was obtained with 12.6 μg/g of BliXyn1 or FveXyn4.v1, however the same increase (7.5 mg/g) could be obtained with a total enzyme concentration of only 6.3 μg/g using a 1:1 mixture of BliXyn1 and FveXyn4.v1 or with a total enzyme concentration of 7.1 μg/g using a 1:8 mixture of BliXyn1 and FveXyn4.v1.

### Example 9

### Performance of BsuGH30 and BliXyn1 after Pepsin Exposure

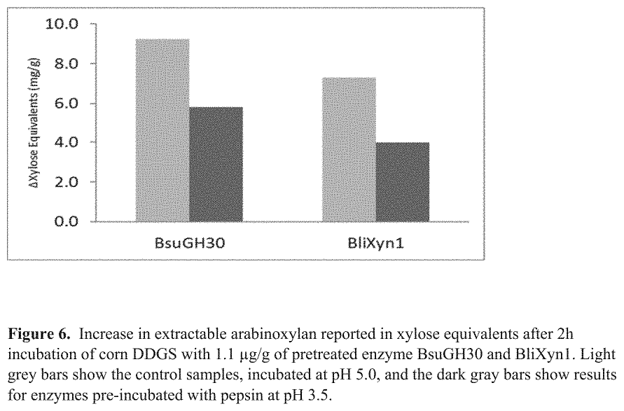

Samples of BsuGH30 and BliXyn1 were incubated with pepsin as described in Example 1 to evaluate their performance after pepsin exposure. FIG. 6 shows the increase in extractable arabinoxylan after incubation of ground corn DDGS with a control enzyme sample, which has been exposed to mild conditions (pH 5.0) and the corresponding enzyme sample, which has been exposed to pepsin, pH 3.5. The tested enzyme inclusion corresponds to 1.1 μg/g corn DDGS. Both BsuGH30 and BliXyn1 maintain the ability to degrade WU-AX from corn DDGS after pepsin exposure, although the performance has decreased.

### Example 10

### Ex Vivo Pig Colon Fermentation Study in the Presence of GH10 and GH30 Enzymes

An increase in hindgut gas production is associated with improved gut health in monogastrics and reflects the stimulation of growth of beneficial bacteria due to an increase in the substrates the bacteria metabolizes. A statistically significant effect (typically >5%) on gas production indicates that test products, such as enzymes added to a feed product are providing a benefit. Another important metric of gut health is the increased production of short-chain fatty acids (SCFA), the major end products of bacterial metabolism in the large intestine, mostly produced by carbohydrate degradation (Macfarlane S., Macfarlane G. T. (2003). Regulation of short-chain fatty acid production. Proceedings of the Nutrition Society. 62 p. 67-72). In the study described below, FveXyn4.v1 alone and FveXyn4.v1 plus BsuGH30 enzymes were tested on pig digesta in an ex vivo and the results are shown below.

Preparation of substrate for ex vivo simulation: Digesta from distal ileum, caecum and proximal colon was collected from pigs fed with corn-based diet containing 5% wheat and 15% corn DDGS. Using high speed centrifugation (18 000×g) the sample was separated in a liquid and solid phase. The liquid phase is stored at −20° C. until use. The solid phase was further washed three times with buffer (pH=5.0) to remove the majority of bacteria present in the digesta and dried at 55° C.

Simulation protocol: The enzyme was dosed based on the amount of dry matter (DM) in the substrate (solid and liquid phase). Table 5 provides the outline for the enzyme dosing. The in-feed enzyme dose per gram of feed was multiplied by a factor 2.2 to compensate for the reduction in DM because of digestion and uptake of easy digestible nutrients (e.g. starch) in the upper digestive tract.

TABLE 5
Treatment overview and enzyme dosing

| | Target enzyme | Enzyme inclusion |
|---|---|---|
| Treatment | dose in feed | rate to digesta |
| Control | none | none |
| FveXyn4.v1 | 2000 XU/kg | 4400 XU/kg DM |
| FveXyn4.v1 + | 2000 XU/kg + | 4400 XU/kg DM + |
| BsuGH30 | 0.8 mg/kg | 1.76 mg/kg DM |

Prior to initiation of the simulation, fresh inoculum was collected from the distal colon of two pigs. In an anaerobic glove box, the inoculum was suspended in substrate's liquid phase and dispensed through a stainless-steel mesh (1 mm). Inoculum, substrate (solid and liquid phase), buffer (pH 6.5) and additive were added in the simulation vessels in an anaerobic chamber. The total volume of the simulation vessels was 15 ml, which contained 0.59 g (0.08 g from liquid phase and 0.51 g from solid phase) substrate-derived dry matter and 1.5% inoculum. The vessels were sealed with thick butyl rubber stoppers, transferred to 37° C. and continuously mixed in a gyratory shaker at 100 rpm. Each of the treatments listed in Table 4 was run in 3 replicates. The incubation was carried out for 18 hours.

Analyzed Parameters:

Bacterial gas production. The total gas production was measured by puncturing the rubber stopper with a needle connected to an accurate 15-ml glass syringe with a sensitive ground plunger. The volume of gas released from the vessels was recorded at 4, 8, 10, 12, 15 and 18-hour simulation and used as a general measure of bacterial activity.

Short-chain fatty acids. At the end of 18-hour simulation 1 ml sub-samples were withdrawn from three replicate vessels by puncturing the butyl rubber stopper with a needle connected to a 1-ml syringe. From these sub-samples, the short-chain fatty acids (SCFAs) were analyzed by gas chromatography, using pivalic acid as an internal standard. Acetic, propionic, and butyric acid were measured.

Statistical analysis consisted of two-tailed t-tests for all measured parameters. The tests were performed against the negative control treatment with no test product amendment. Significance according to Student's t-test: p-value <0.05* and p-value <0.01**.

The results of this ex vivo pig fermentation studies are summarized on Table 6 and Table 7. As shown on Table 6, the combination of FveXyn4.v1 and BsuGH30 increased the microbial gas formation significantly, whereas the inclusion of only FveXyn4.v1 did not.

TABLE 6
Microbial gas production

| | GAS (gas released during timeframes listed | | | | | |
|---|---|---|---|---|---|---|
| | (measured in mL) | | | | | |
| | 0-4 | 0-8 | 0-10 | 0-12 | 0-15 | 0-18 |
| | h | h | h | h | h | h |
| Control | 4.3 | 7.3 | 7.9 | 8.6 | 8.8 | 9.8 |
| FveXyn4.v1 | 4.7 | 7.7 | 8.1 | 8.8 | 9.1 | 10 |
| FveXyn4.v1 + | 4.2 | 7.8** | 8.6** | 9.4** | 9.7** | 10.7* |
| BsuGH30 | | | | | | |

TABLE 7
SCFA production after 18 hours

| | Acetic acid | Propionic acid | Butyric acid |
|---|---|---|---|
| | (mM) | (mM) | (mM) |
| Control | 40.9 | 23.9 | 15.4 |
| FveXyn4.v1 | 41.1 | 25.7 | 16.4* |
| FveXyn4.v1 + BsuGH30 | 47.1** | 28.2* | 17.3* |

In addition, after 18 hours of incubation, a considerable increase in the production of acetic, propionic and butyric acids was observed with inclusion of the combination of FveXyn4.v1 and BsuGH30 enzymes when compared to the control (no enzyme). In contrast, FveXyn4.v1 alone, only yielded an increase in butyric acid rise that was statistically significant (Table 7).

### Example 11

### Comparison of Glucuronoxylanase Sequences

Related proteins were identified by a BLAST search (Altschul et al., Nucleic Acids Res, 25-3389-402, 1997) using the mature amino acid sequences for BsuGH30 (SEQ ID NO:29); BliXyn1 (SEQ ID NO:30); BamGh2 (SEQ ID NO:31); BsaXyn1 (SEQ ID NO:32); PmaXyn4 (SEQ ID NO:33); PcoXyn1 (SEQ ID NO:34); and PtuXyn2 (SEQ ID NO:35) against Public and Genome Quest Patent databases with search parameters set to default values and a subset are shown on Tables 8A and 8B (BsuGH30); Tables 9A and 9B (BliXyn1); Tables 10A and 10B (BamGh2); Tables 11A and 11B (BsaXyn1); Tables 12A and 12B (PmaXyn4); Tables 13A and 13B (PcoXyn1); and Tables 14A and 14B (PtuXyn2) respectively. Percent identity (PID) for both search sets is defined as the number of identical residues divided by the number of aligned residues in the pairwise alignment. Value labeled “Sequence length” on tables corresponds to the length (in amino acids) for the proteins referenced with the listed Accession numbers, while “Aligned length” refers to sequence used for alignment and PID calculation.

TABLE 8A
List of sequences with percent identity to BsuGH30 mature
protein identified from the NCBI non-redundant protein database

| | | | Sequence | Alignment |
|---|---|---|---|---|
| Accession # | PID | Organism | Length | Length |
| WP_003231534.1 | 100.0 | Bacillus subtilis | 422 | 390 |
| WP_041521354.1 | 99.2 | Bacillus sp. JS | 422 | 390 |
| WP_072566343.1 | 98.5 | Bacillus subtilis | 422 | 390 |
| WP_071579318.1 | 98.2 | Bacillus sp. FMQ74 | 422 | 390 |
| WP_024573168.1 | 98.2 | Bacillus subtilis | 422 | 390 |
| WP_014476954.1 | 97.7 | Bacillus subtilis | 422 | 390 |
| WP_040083023.1 | 97.9 | Bacillus sp. A053 | 422 | 390 |
| WP_086343892.1 | 98.2 | Bacillus subtilis | 422 | 390 |
| WP_060398719.1 | 95.9 | Bacillus subtilis | 423 | 390 |
| WP_075746411.1 | 95.4 | Bacillus licheniformis | 423 | 390 |
| WP_064814128.1 | 95.1 | Bacillus subtilis | 423 | 390 |

TABLE 8B
List of sequences with percent identity to BsuGH30 mature protein
identified from Genome Quest database

| | | | | Align. |
|---|---|---|---|---|
| GQ Identifier | PID | Organism | Length | length |
| BCM03707 | 100.00 | Bacillus subtilis | 390 | 390 |
| US20160354436-0659 | 94.36 | Bacillus | 394 | 390 |
| | | amyloliquefaciens | | |
| AZG68558 | 93.85 | Unidentified | 423 | 390 |
| KR1020160056941- | 93.08 | Unidentified | 390 | 390 |
| 0659 | | | | |
| US20160040203-0020 | 92.31 | Bacillus atrophaeus | 389 | 388 |
| US20160040203-0025 | 91.28 | Bacillus | 389 | 388 |
| | | amyloliquefaciens | | |
| US20160339078-5040 | 91.03 | Bacillus subtilis | 423 | 390 |
| US20160040203-0024 | 90.77 | Bacillus licheniformis | 389 | 388 |
| US20160040203-0016 | 86.92 | Geobacillus sp. | 389 | 389 |
| AZG68554 | 86.41 | Unidentified | 426 | 390 |
| US20160040203-0021 | 85.64 | Bacillus stratosphericus | 387 | 389 |
| US20160040203-0023 | 85.64 | Bacillus pumilus | 388 | 388 |
| WO2017103159-0012 | 85.38 | Paenibacillus sp-19179 | 391 | 390 |
| US20160040203-0022 | 84.36 | Bacillus pumilus | 388 | 388 |
| US20160040203-0019 | 84.10 | Bacillus xiamenensis | 388 | 389 |

TABLE 9A
List of sequences with percent identity to BliXyn1 mature protein
identified from the NCBI non-redundant protein database

| | | | Sequence | Alignment |
|---|---|---|---|---|
| Accession # | PID | Organism | Length | Length |
| KFM84586.1 | 100.0 | Bacillus | 420 | 391 |
| | | paralicheniformis | | |
| WP_044803292.1 | 99.7 | Bacillus licheniformis | 407 | 391 |
| WP_048353311.1 | 94.1 | Bacillus | 423 | 389 |
| | | glycinifermentans | | |
| WP_051290454.1 | 93.9 | Bacillus | 407 | 391 |
| WP_060398719.1 | 93.6 | Bacillus subtilis | 423 | 389 |
| WP_084992328.1 | 93.6 | Bacillus subtilis | 423 | 389 |
| WP_088110831.1 | 93.6 | Bacillus subtilis | 423 | 389 |

TABLE 9B
List of sequences with percent identity to BliXyn1
mature protein identified from Genome Quest database

| | | | | Align. |
|---|---|---|---|---|
| GQ Identifier | PID | Organism | Length | length |
| US20160040203-0024 | 98.98 | Bacillus licheniformis | 389 | 388 |
| AZG68558 | 92.58 | Unidentified | 423 | 391 |
| US20160354436-0659 | 91.56 | Bacillus | 394 | 391 |
| | | amyloliquefaciens | | |
| BCM03707 | 91.30 | Bacillus subtilis | 390 | 391 |
| US20160040203-0020 | 91.05 | Bacillus atrophaeus | 389 | 388 |
| KR1020160056941- | 90.28 | Unidentified | 390 | 392 |
| 0659 | | | | |
| US20160339078-5040 | 89.77 | Bacillus subtilis | 423 | 391 |
| US20160040203-0025 | 89.51 | Bacillus | 389 | 388 |
| | | amyloliquefaciens | | |
| AZG68554 | 89.26 | Unidentified | 426 | 392 |
| US20160040203-0016 | 89.00 | Geobacillus sp. | 389 | 388 |
| WO2017103159-0012 | 87.72 | Paenibacillus sp-19179 | 391 | 391 |
| US20160040203-0021 | 85.42 | Bacillus stratosphericus | 387 | 389 |
| US20160040203-0023 | 84.65 | Bacillus pumilus | 388 | 388 |
| US20160040203-0019 | 84.40 | Bacillus xiamenensis | 388 | 389 |

TABLE 10A
List of sequences with percent identity to BamGh2 mature protein
identified from the NCBI non-redundant protein database

| | | | Sequence | Alignment |
|---|---|---|---|---|
| Accession # | PID | Organism | Length | Length |
| WP_039251362.1 | 100.0 | Bacillus | 423 | 390 |
| WP_077391892.1 | 99.7 | Bacillus sp. 275 | 423 | 390 |
| WP_043021156.1 | 99.5 | Bacillus velezensis | 423 | 390 |
| WP_015417568.1 | 99.2 | Bacillus velezensis | 423 | 390 |
| WP_029325938.1 | 99.2 | Bacillus | 423 | 390 |
| WP_064107346.1 | 99.2 | Bacillus velezensis | 423 | 390 |
| WP_071181797.1 | 99.0 | Bacillus velezensis | 423 | 390 |
| WP_015388168.1 | 99.0 | Bacillus | 423 | 390 |
| | | amyloliquefaciens | | |
| WP_032865984.1 | 99.0 | Bacillus | 423 | 390 |
| | | amyloliquefaciens | | |
| WP_053285110.1 | 98.7 | Bacillus velezensis | 423 | 390 |
| WP_073982051.1 | 98.7 | Bacillus | 423 | 390 |
| | | amyloliquefaciens | | |
| WP_046559614.1 | 98.5 | Bacillus | 423 | 390 |
| WP_033574822.1 | 98.7 | Bacillus | 423 | 390 |
| | | amyloliquefaciens | | |
| WP_070081901.1 | 98.2 | Bacillus | 423 | 390 |
| WP_064778982.1 | 95.1 | Bacillus siamensis | 423 | 390 |
| WP_071346697.1 | 95.1 | Bacillus | 423 | 390 |
| | | amyloliquefaciens | | |
| WP_065521198.1 | 94.9 | Bacillus | 423 | 390 |
| | | amyloliquefaciens | | |
| WP_060962668.1 | 94.9 | Bacillus sp. SDLI1 | 423 | 390 |
| WP_065981591.1 | 94.6 | Bacillus | 423 | 390 |
| | | amyloliquefaciens | | |
| WP_024716468.1 | 94.6 | Bacillus tequilensis | 423 | 390 |

TABLE 10B
List of sequences with percent identity to BamGh2 mature
protein identified from Genome Quest database

| | | | | Align. |
|---|---|---|---|---|
| GQ Identifier | PID | Organism | Length | length |
| US20160339078- | 99.23 | Bacillus subtilis | 423 | 390 |
| 5040 | | | | |
| US20160040203- | 98.72 | Bacillus | 389 | 388 |
| 0025 | | amyloliquefaciens | | |
| US20160339078- | 94.62 | Bacillus | 423 | 390 |
| 4253 | | amyloliquefaciens | | |
| KR1020160056941- | 93.33 | Unidentified | 390 | 390 |
| 0659 | | | | |
| AZG68558 | 92.82 | Unidentified | 423 | 390 |
| US20160040203- | 92.82 | Bacillus atrophaeus | 389 | 388 |
| 0020 | | | | |
| BCM03707 | 91.28 | Bacillus subtilis | 390 | 390 |
| US20160040203- | 89.74 | Bacillus licheniformis | 389 | 388 |
| 0024 | | | | |
| US20160040203- | 88.21 | Bacillus pumilus | 388 | 388 |
| 0023 | | | | |
| US20160040203- | 86.92 | Bacillus stratosphericus | 387 | 388 |
| 0021 | | | | |
| US20160040203- | 86.67 | Bacillus pumilus | 388 | 388 |
| 0022 | | | | |
| US20160040203- | 85.38 | Bacillus xiamenensis | 388 | 388 |
| 0019 | | | | |
| US20160040203- | 84.87 | Geobacillus sp. | 389 | 388 |
| 0016 | | | | |
| AZG68554 | 84.62 | Unidentified | 426 | 390 |
| AZG68560 | 84.62 | Unidentified | 401 | 383 |

TABLE 11A
List of sequences with percent identity to BsaXyn1 mature protein
identified from the NCBI non-redundant protein database

| | | | Sequence | Alignment |
|---|---|---|---|---|
| Accession # | PID | Organism | Length | Length |
| WP_060596459.1 | 100.0 | Bacillus pumilus | 422 | 389 |
| WP_034322788.1 | 98.5 | Bacillus | 422 | 389 |
| | | zhangzhouensis | | |
| WP_034660820.1 | 99.2 | Bacillus pumilus | 421 | 389 |
| WP_034619861.1 | 98.5 | Bacillus pumilus | 422 | 389 |
| WP_044141361.1 | 97.9 | Bacillus pumilus | 422 | 389 |
| WP_041117582.1 | 97.4 | Bacillus pumilus | 421 | 389 |
| WP_050944862.1 | 97.7 | Bacillus pumilus | 421 | 389 |
| WP_041815581.1 | 96.9 | Bacillus pumilus | 422 | 389 |
| WP_056766672.1 | 96.9 | Bacillus sp. Root920 | 421 | 389 |
| WP_058015629.1 | 96.9 | Bacillus pumilus | 421 | 389 |
| WP_060697980.1 | 96.4 | Bacillus australimaris | 421 | 389 |
| WP_024719061.1 | 95.6 | Bacillus | 421 | 389 |
| WP_083693004.1 | 95.4 | Bacillus sp. RRD69 | 422 | 389 |
| WP_081832196.1 | 95.4 | Bacillus sp. | 422 | 389 |
| | | UNC125MFCrub1.1 | | |
| WP_082627046.1 | 95.1 | Bacillus sp. TH007 | 422 | 389 |
| WP_081038966.1 | 95.1 | Bacillus | 421 | 389 |
| WP_082136042.1 | 95.1 | Bacillus altitudinis | 421 | 389 |
| OQP20089.1 | 94.9 | Bacillus | 420 | 389 |
| | | stratosphericus | | |

TABLE 11B
List of sequences with percent identity to BsaXyn1 mature
protein identified from Genome Quest database

| | | | | Align. |
|---|---|---|---|---|
| GQ Identifier | PID | Organism | Length | length |
| US20160040203-0023 | 97.94 | Bacillus pumilus | 388 | 387 |
| US20160040203-0022 | 96.40 | Bacillus pumilus | 388 | 387 |
| US20160040203-0021 | 95.12 | Bacillus stratosphericus | 387 | 387 |
| US20160040203-0019 | 92.54 | Bacillus xiamenensis | 388 | 387 |
| AZG68560 | 92.29 | Unidentified | 401 | 370 |
| US20160339078-5040 | 89.72 | Bacillus subtilis | 423 | 390 |
| US20160040203-0025 | 89.46 | Bacillus | 389 | 388 |
| | | amyloliquefaciens | | |
| AZG68558 | 89.46 | Unidentified | 423 | 390 |
| US20160040203-0020 | 88.69 | Bacillus atrophaeus | 389 | 388 |
| US20160339078-4253 | 87.92 | Bacillus | 423 | 390 |
| | | amyloliquefaciens | | |
| BCM03707 | 87.40 | Bacillus subtilis | 390 | 390 |
| KR1020160056941- | 86.63 | Unidentified | 390 | 390 |
| 0659 | | | | |
| US20160040203-0024 | 86.12 | Bacillus licheniformis | 389 | 388 |

TABLE 12A
List of sequences with percent identity to PmaXyn4 mature protein
identified from the NCBI non-redundant protein database

| | | | Sequence | Alignment |
|---|---|---|---|---|
| Accession # | PID | Organism | Length | Length |
| WP_036622637.1 | 100.0 | Paenibacillus macerans | 447 | 417 |
| OMG45831.1 | 99.8 | Paenibacillus macerans | 447 | 417 |
| ACX65526.1 | 94.6 | Paenibacillus sp. | 426 | 391 |
| | | Y412MC10 | | |
| WP_041622197.1 | 94.6 | Paenibacillus sp. | 422 | 391 |
| | | Y412MC10 | | |
| ETT66763.1 | 94.4 | Paenibacillus sp. | 426 | 391 |
| | | FSL H8-457 | | |
| WP_036660965.1 | 94.4 | Paenibacillus sp. | 422 | 391 |
| | | FSL H8-457 | | |

TABLE 12B
List of sequences with percent identity to PmaXyn4 mature
protein identified from Genome Quest database

| GQ Identifier | PID | Organism | Length | Align. length |
|---|---|---|---|---|
| AZG68554 | 88.73 | Unidentified | 426 | 417 |
| US20160040203- | 88.25 | Geobacillus sp. | 389 | 388 |
| 0016 | | | | |
| WO2017103159- | 87.77 | Paenibacillus sp-19179 | 391 | 391 |
| 0012 | | | | |
| US20160040203- | 82.49 | Bacillus licheniformis | 389 | 388 |
| 0024 | | | | |
| US20160339078- | 81.77 | Bacillus subtilis | 422 | 417 |
| 5088 | | | | |
| AZG68558 | 81.77 | Unidentified | 423 | 417 |

TABLE 13A
List of sequences with percent identity to PcoXyn1 mature protein
identified from the NCBI non-redundant protein database

| | | | Sequence | Alignment |
|---|---|---|---|---|
| Accession # | PID | Organism | Length | Length |
| KHF34457.1 | 99.3 | Paenibacillus sp. | 433 | 418 |
| | | P1XP2 | | |
| AET60095.1 | 80.3 | Paenibacillus terrae | 557 | 522 |
| | | HPL-003 | | |
| WP_085979683.1 | 80.3 | Paenibacillus terrae | 536 | 522 |

TABLE 13B
List of sequences with percent identity to PcoXyn1
mature protein identified from Genome Quest database

| GQ | | | | |
|---|---|---|---|---|
| Identifier | PID | Organism | Length | Align. length |
| AZG68556 | 78.31 | Unidentified | 564 | 529 |
| AAW69963 | 75.62 | Aeromonas punctata | 528 | 518 |

TABLE 14A
List of sequences with percent identity to PtuXyn2 mature protein
identified from the NCBI non-redundant protein database

| | | | | Align- |
|---|---|---|---|---|
| | | | Sequence | ment |
| Accession # | PID | Organism | Length | Length |
| WP_063567972.1 | 99.0 | Paenibacillus sp. O199 | 422 | 392 |
| WP_064640831.1 | 99.0 | Paenibacillus sp. AD87 | 422 | 392 |
| OAX48465.1 | 99.0 | Paenibacillus sp. AD87 | 423 | 392 |
| WP_079693657.1 | 98.7 | Paenibacillus sp. RU5A | 422 | 392 |
| WP_072733029.1 | 98.7 | Paenibacillus sp. ov031 | 422 | 392 |
| SEN81008.1 | 97.7 | Paenibacillus sp. OK076 | 423 | 392 |
| WP_062319325.1 | 93.9 | Paenibacillus pabuli | 425 | 391 |

TABLE 14B
List of sequences with percent identity to PtuXyn2 mature
protein identified from Genome Quest database

| GQ Identifier | PID | Organism | Length | Align. length |
|---|---|---|---|---|
| AAW69963 | 84.95 | Aeromonas punctata | 528 | 392 |
| AZG68556 | 84.95 | Unidentified | 564 | 392 |
| US20160040203- | 84.69 | Paenibacillus sp. | 390 | 389 |
| 0013 | | | | |

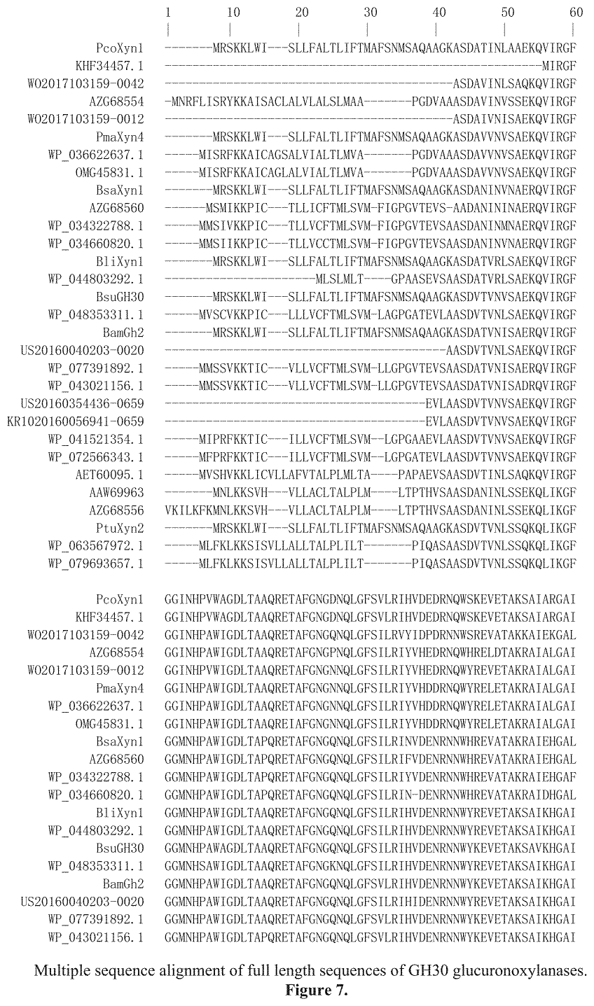

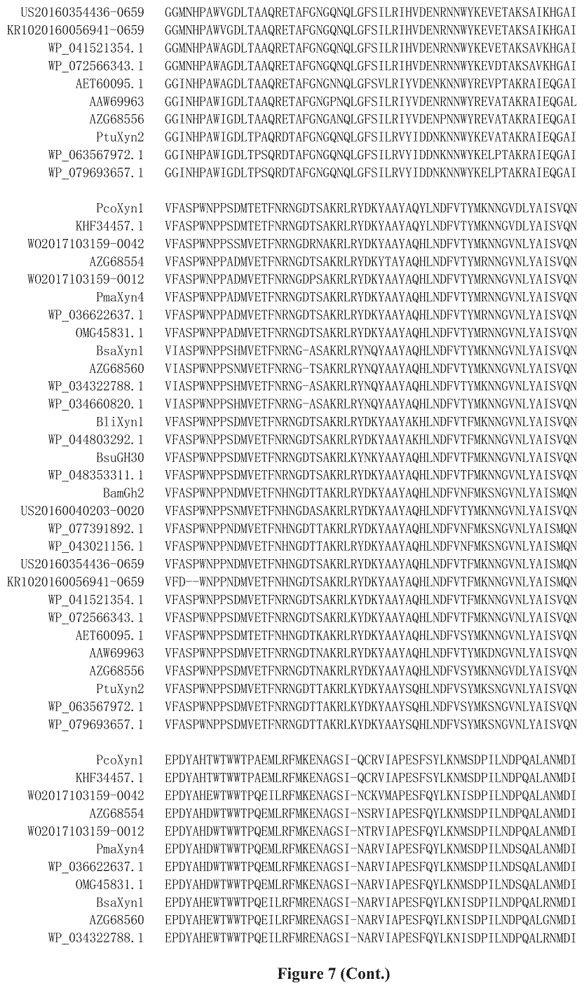

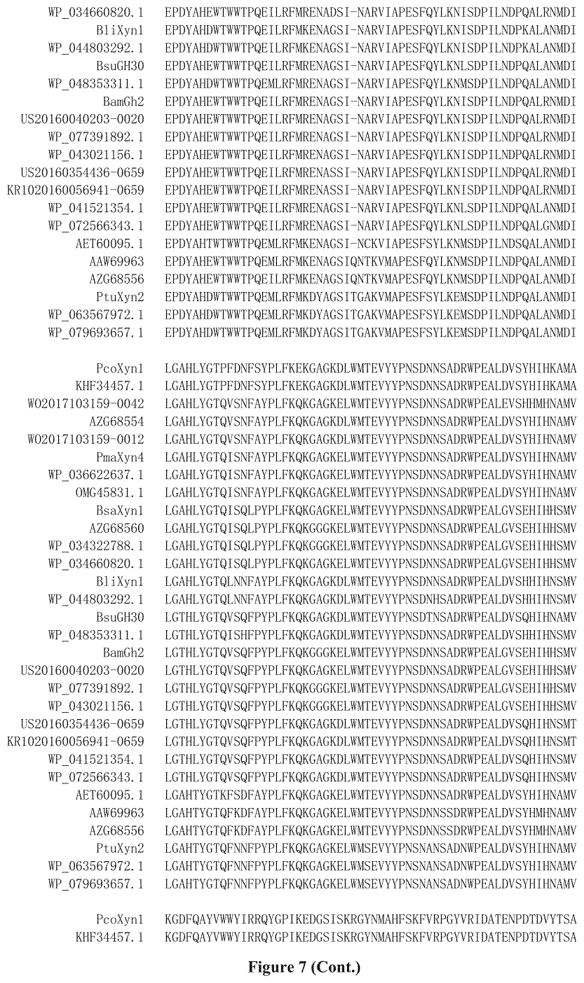

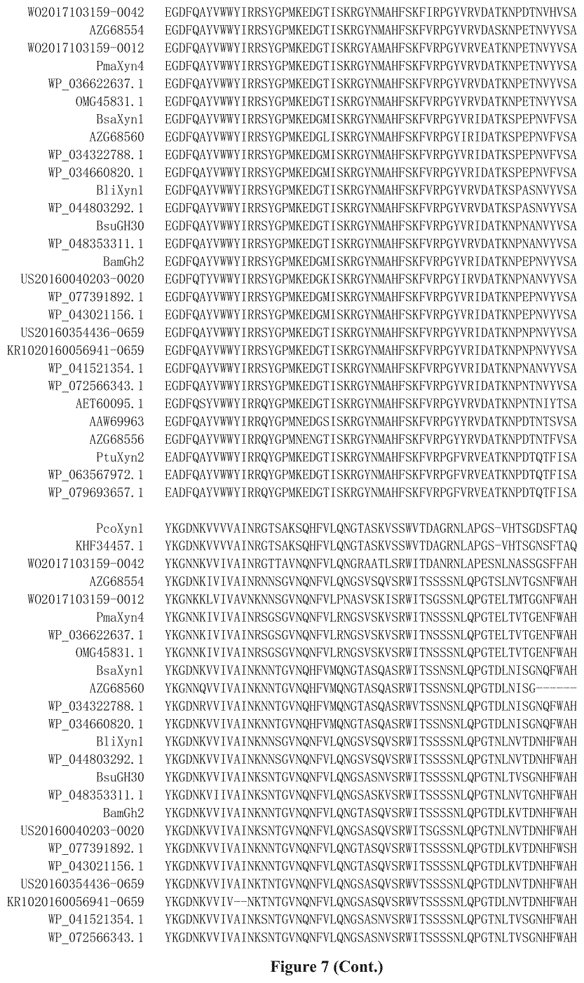

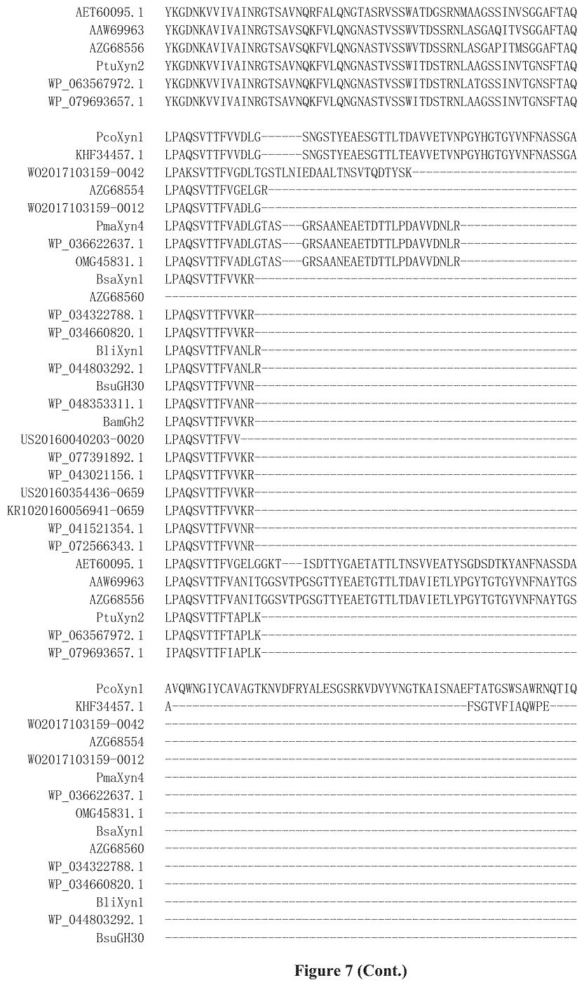

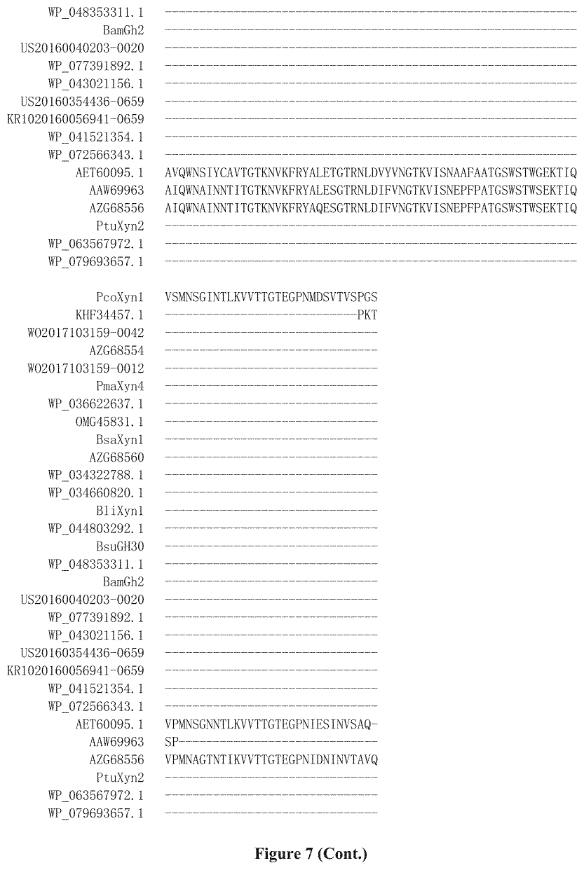

The amino acid sequences for the full-length proteins BsuGH30 (SEQ ID NO:2); BliXyn1 (SEQ ID NO: 4); BamGh2 (SEQ ID NO:6); BsaXyn1 (SEQ ID NO:8); PmaXyn4 (SEQ ID NO: 10); PcoXyn1 (SEQ ID NO: 12); and PtuXyn2 (SEQ ID NO: 14), and the sequences of other GH30 xylanases from Tables 3-9 were aligned with default parameters using the MUSCLE program from Geneious software (Biomatters Ltd.) (Robert C. Edgar. MUSCLE: multiple sequence alignment with high accuracy and high throughput Nucl. Acids Res. (2004) 32 (5): 1792-1797). The multiple sequence alignment is shown on FIG. 7. The percent identity of the mature amino acid sequences of the GH30 glucuronoxylanases is shown in Table 15.

TABLE 15
Percent sequence identity among mature amino acid sequences of GH30 enzymes

| | BamGh2 | BsaXyn1 | BsuGH30 | BliXyn1 | PmaXyn4 | PcoXyn1 | PtuXyn2 |
|---|---|---|---|---|---|---|---|
| BamGh2 | | 89.5 | 91.3 | 90.0 | 84.4 | 78.2 | 76.5 |
| BsaXyn1 | 89.5 | | 87.2 | 86.2 | 82.6 | 76.7 | 74.4 |
| BsuGH30 | 91.3 | 87.2 | | 91.3 | 86.7 | 80.3 | 78.3 |
| BliXyn1 | 90.0 | 86.2 | 91.3 | | 88.5 | 79.8 | 79.1 |
| PmaXyn4 | 84.4 | 82.6 | 86.7 | 88.5 | | 78.4 | 79.3 |
| PcoXyn1 | 78.2 | 76.7 | 80.3 | 79.8 | 78.4 | | 77.8 |
| PtuXyn2 | 76.5 | 74.4 | 78.3 | 79.1 | 79.3 | 77.8 | |

What is claimed is:

1. An additive for animal feed comprising corn or rice, said feed additive comprising at least one enzyme having glucuronoxylanase activity and at least one enzyme having endo-beta-1,4-xylanase activity wherein degradation of insoluble glucuronoxylan is greater than if either enzyme was used alone.

2. A feed additive comprising at least one enzyme with glucuronoxylanase activity and at least one enzyme having endo-beta-1,4-xylanase activity wherein said combination is better in stimulating growth of beneficial bacteria in a digestive tract of a monogastric animal fed a corn based diet when compared to the use of the xylanase having endo-beta-1,4-xylanase activity alone.

3. A feed additive comprising at least one enzyme with glucuronoxylanase activity and at least one enzyme having endo-beta-1,4-xylanase activity wherein said combination is capable of increasing production of at least one short chain fatty acid in a monogastric animal fed a corn based diet when compared to the use of the xylanase having endo-beta-1,4-xylanase activity alone.

4. The feed additive of claim 3 wherein the short chain fatty acid is selected from the group consisting of acetic acid, propionic acid or butyric acid.

5. The additive of any one of claims 1-4, wherein the xylanase having glucuronoxylanase activity is a GH30 glucuronoxylanase.

6. The additive of claim 5, wherein the xylanase having glucuronoxylanase activity is derived from Bacillus or Paenibacillus sp.

7. The additive of claim 5 or claim 6, wherein the xylanase having glucuronoxylanase activity is derived from B. subtilis or B. licheniformis.

8. The additive composition of claim 6, wherein the xylanase having glucuronoxylanase activity comprises a polypeptide having at least 90% sequence identity to a polypeptide selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, and SEQ ID NO:42.

9. The additive of claim 8, wherein the xylanase having glucuronoxylanase activity comprises a polypeptide selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, and SEQ ID NO:42.

10. The additive of any one of claims 1-9, wherein the xylanase having endo-beta-1,4-xylanase activity is derived from a filamentous fungus.

11. The additive of claim 10, wherein the xylanase having endo-beta-1,4-xylanase activity comprises a polypeptide having at least 90% sequence identity to a polypeptide selected from the group consisting of SEQ ID NO:46, SEQ ID NO:47, SEQ ID NO:48, and SEQ ID NO:52.

12. The additive of any one of claims 1-11, wherein at least one of the xylanases is recombinantly produced.

13. The additive of any claim 1-12, which further comprises (a) one or more of the enzymes selected the group consisting of an amylase, protease, endo-glucanase and phytase; or (b) one or more direct fed microbials or (c) a combination of (a) and (b).

14. A premix comprising the additive of any one of claims 1-13, and at least one vitamin and/or mineral.

15. A corn or rice-based animal feed comprising at least one enzyme with glucuronoxylanase activity and at least one enzyme having endo-beta-1,4-xylanase activity wherein degradation of insoluble glucuronoxylan is greater than if either enzyme was used alone.

16. A corn-based animal feed comprising at least one enzyme with glucuronoxylanase activity and at least one enzyme having endo-beta-1,4-xylanase activity wherein said combination is better in stimulating growth of beneficial bacteria in a digestive tract of a monogastric animal when compared to the use of the xylanase having endo-beta-1,4-xylanase activity alone.

17. A corn-based animal feed comprising at least one enzyme with glucuronoxylanase activity and at least one enzyme having endo-beta-1,4-xylanase activity wherein said combination is capable of increasing production of at least one short chain fatty acid in a monogastric animal when compared to the use of the xylanase having endo-beta-1,4-xylanase activity alone.

18. The animal feed of claim 17, wherein the short chain fatty acid is selected from the group consisting of acetic acid, propionic acid or butyric acid.

19. The animal feed of any one of claims 15-18, wherein the xylanase having glucuronoxylanase activity is a GH30 glucuronoxylanase.

20. The animal feed of claim 19, wherein the xylanase having glucuronoxylanase activity is derived from Bacillus or Paenibacillus sp.

21. The animal feed of claim 19 or claim 20, wherein the xylanase having glucuronoxylanase activity is derived from B. subtilis or B. licheniformis.

22. The animal feed of claim 20, wherein the xylanase having glucuronoxylanase activity comprises a polypeptide having at least 90% sequence identity to a polypeptide selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, and SEQ ID NO:42.

23. The animal feed of claim 22, wherein the xylanase having glucuronoxylanase activity comprises a polypeptide selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, and SEQ ID NO:42.

24. The animal feed of any one of claims 15-23, wherein the xylanase having endo-beta-1,4-xylanase activity is derived from a filamentous fungus.

25. The animal feed of claim 24, wherein the xylanase having endo-beta-1,4-xylanase activity comprises a polypeptide having at least 90% sequence identity to a polypeptide selected from the group consisting of SEQ ID NO:46, SEQ ID NO:47, SEQ ID NO:48, and SEQ ID NO:52.

26. The animal feed of any one of claims 15-25, wherein at least one of the xylanases is recombinantly produced.

27. The animal feed of claim 15-26, which further comprises (a) one or more of the enzymes selected the group consisting of an amylase, protease, endo-glucanase and phytase; (b) one or more direct fed microbials or (c) a combination of (a) and (b).

28. A method for degrading insoluble glucuronoxylan in an animal feed comprising corn or rice comprising contacting the corn or rice with at least one enzyme with glucuronoxylanase activity and at least one enzyme having endo-beta-1,4-xylanase activity.

29. A method for improving the digestibility of insoluble glucuronoxylan in a corn or rice-based animal feed comprising administering to an animal a corn or rice-based animal feed comprising at least one enzyme with glucuronoxylanase activity and at least one enzyme having endo-beta-1,4-xylanase activity.

30. The method of claim 28 or claim 29, wherein the xylanase having glucuronoxylanase activity is a GH30 glucuronoxylanase.

31. The method of claim 30, wherein the xylanase having glucuronoxylanase activity is derived from Bacillus or Paenibacillus sp.

32. The method of claim 30 or claim 31, wherein the xylanase having glucuronoxylanase activity is derived from B. subtilis or B. licheniformis.

33. The method of claim 31, wherein the xylanase having glucuronoxylanase activity comprises a polypeptide having at least 90% sequence identity to a polypeptide selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, and SEQ ID NO:42.

34. The method of claim 33, wherein the xylanase having glucuronoxylanase activity comprises a polypeptide selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, and SEQ ID NO:42.

35. The method of any one of claims 28-34, wherein the xylanase having endo-beta-1,4-xylanase activity is derived from a filamentous fungus.

36. The method of claim 35, wherein the xylanase having endo-beta-1,4-xylanase activity comprises a polypeptide having at least 90% sequence identity to a polypeptide selected from the group consisting of SEQ ID NO:46, SEQ ID NO:47, SEQ ID NO:48, and SEQ ID NO:52.

37. The method of any one of claims 28-36, wherein at least one of the xylanases is recombinantly produced.

38. The method of any one of claims 28-37, further comprising administering to the animal (a) one or more of the enzymes selected the group consisting of an amylase, protease, endo-glucanase and phytase; (b) one or more direct fed microbials; or (c) a combination of (a) and (b).

39. The method of any one of claims 28-38, wherein the animal is a monogastric animal selected from the group consisting of pigs and swine, turkeys, ducks, chicken, salmon, trout, tilapia, catfish, carp, shrimps and prawns.

40. The method of any one of claims 28-38, wherein the animal is a ruminant animal selected from the group consisting of cattle, young calves, goats, sheep, giraffes, bison, moose, elk, yaks, water buffalo, deer, camels, alpacas, llamas, antelope, pronghorn and nilgai.
